(19) United States
(12) Patent Application Publication
(10) Pub. No.: US 2018/0299251 A1
(43) Pub. Date: Oct. 18, 2018

(54) METHODS AND APPARATUS FOR SPECKLE-FREE OPTICAL COHERENCE IMAGING

(71) Applicant: The Board of Trustees of the Leland Stanford Junior University, Stanford, CA (US)

(72) Inventors: Orly LIBA, Stanford, CA (US); Matthew D. LEW, St. Louis, MO (US); Elliott D. SORELLE, Stanford, CA (US); Adam DE LA ZERDA, Palo Alto, CA (US)

(73) Assignee: The Board of Trustees of the Leland Stanford Junior University, Stanford, CA (US)

(21) Appl. No.: 15/768,708

(22) PCT Filed: Oct. 19, 2016
(86) PCT No.: PCT/US16/57656

§ 371 Date: Apr. 16, 2018

Related U.S. Application Data
(60) Provisional application No. 62/243,466, filed on Oct. 19, 2015.

(51) Int. Cl. G01B 9/02; A61B 3/10; A61B 3/12; A61B 5/00; G02B 27/48; G01N 21/47

(52) U.S. Cl. CPC G01B 9/02091; A61B 3/102; A61B 3/1225; A61B 5/0066; G01N 21/4795; A61B 5/7203; G01B 9/0201; G01B 9/02082; G02B 27/48; A61B 5/0073

(57) ABSTRACT

An apparatus includes a light splitter to receive a light beam and direct a first portion of the light beam to a reference arm and a second portion of the light beam to a sample arm. The sample arm includes a phase scrambler, in a path of the second portion of the light beam, to cause local-random-time varying phase modulation to the second portion of the light beam. The sample arm also includes a controller to change the local phase of the second portion of the light. The apparatus further includes a detector, in optical communication with the reference arm and the sample arm, to detect an interference pattern produced by the first portion of the light beam propagated through the reference arm and the second portion of the light beam scattered from the sample via the sample arm.

### CROSS-REFERENCE TO RELATED APPLICATIONS

This application benefit of priority to U.S. Provisional Application Ser. No. 62/243,466, entitled “Methods and Apparatus for Speckle-Free Optical Coherence Imaging,” filed Oct. 19, 2015, which is incorporated herein by reference in its entirety.

### GOVERNMENT SUPPORT

This invention was made with Government support under contracts NSF 1438340 awarded by National Science Foundation, CA 151459 awarded by the National Cancer Institute and OD 012179 awarded by the National Institutes of Health. The Government has certain rights in the invention.

### BACKGROUND

Optical Coherence Tomography (OCT) is an interference-based imaging technique. Known OCT systems and methods typically employ a reference arm and a sample arm. The sample arm delivers light to a sample to be imaged and collects light scattered or diffused from the sample. The scattered sample light is then mixed with light that is reflected from the reference arm (the “reference light”). If the sample light and the reference light are coherent, the mixing can produce an interference pattern that can be detected and then converted to an image. The coherence between the sample light and the reference light can be achieved by closely matching the path lengths of the sample and reference arms. Generally, the reference arm can be adjusted (e.g., changing the position of the reflector in the reference arm) to match the path lengths.

OCT can be useful in acquiring cross-sectional (tomographic) or volumetric images of a sample by scanning light across the sample. An image produced along the depth of the sample is conventionally termed as an A-scan. Each A-scan can provide information about the reflective or scattering properties of the sample as a function of depth at one position of the scanned beam. A cross-sectional image of the sample can be produced by combining neighboring A-scans. A volumetric image can be constructed from a group of B-scans, each of which can be an image of a planar slice into the sample. A B-scan, however, does not have to be a planar image. The B-scan can also be an image along a circle, and this cross-sectional view is then an annular scan around a point of interest in the sample.

Typically, at least three types of volumetric images are used in OCT imaging. A series of parallel B-scans can produce a rectangular, or raster volume scan; a series of B-scans at regular angular intervals can produce a radial volume scan; and an annular volume scan can be produced by a series of B-scans forming concentric rings. Each type of volumetric image can have its own advantages in particular circumstances. For example, rectangular volumes are frequently used in imaging the macula. Rectangular or annular scans are often used in the vicinity of the optic nerve head. Radial scans are often used in imaging the cornea.

OCT can have several desirable properties in imaging. First, the depth resolution, which can be dependent on precision of the depth scanning, can be independent from transverse resolution. High depth resolution can be achieved even at sites that may be not accessible by high numerical aperture (NA) beams, such as the fundus of the eye. A practical range of depth resolution can be on the order of 1 μm. Second, the interferometric technique used in OCT can provide high dynamic range and sensitivity (>100 dB), which can be beneficial in imaging of weakly scattering structures even in a scattering environment, thereby allowing “in situ optical biopsy.” Third, OCT is typically non-invasive and therefore can produce in vivo data without causing damages to the sample.

One issue with coherent imaging techniques, including OCT, however, is speckle noise. Speckle noise can be caused by the interference of light scattering from multiple points within a volume (or three-dimensional space) of the sample where light is focused and from which it is collected (this volume can be referred to as a resolution volume, a volumetric pixel or a voxel). More specifically, most surfaces, synthetic or natural, are rough on micro-scales (e.g., on the scale of the optical wavelengths). The rough surface (more specifically the reflectivity function of the surface) can be modeled as a collection of scatterers, each of which can scatter incident light. Because of the finite spatial resolution of an imaging system, at any time the light received by the detector can be regarded as being from a distribution of scatterers within the resolution volume. The scattered light adds coherently, i.e., the light from the scatterers interacts constructively and destructively depending on the relative phases of each scattered waveform. Constructive and destructive interference creates bright and dark dots in the image, thereby creating speckle noise, which can reduce the contrast of the resulting image thereby making boundaries between certain structures difficult to resolve.

Some known OCT systems and methods attempt to reduce speckle noise by employing incoherent averaging (also referred to as compounding) of several images (also referred to as snapshot). For example, averaging M images with uncorrelated speckle noise can reduce the speckle contrast by (M)^{1/2}. The speckle contrast can be defined as the standard deviation of the noise divided by the mean intensity. Non-correlated speckle patterns can be obtained by various methods, including, but are not limited to, scanning from different angles, scanning several nearby regions, scanning with different incident wavelengths, and scanning with different polarizations. These methods can be referred to as angular, spatial, frequency, and polarization compounding, respectively. Compounding methods, however, normally compromise the resolution or depth of field of the resulting image when further reduction of speckle noise is pursued. Therefore, it can be challenging for compounding methods to eliminate speckle noise entirely.

Other known OCT systems and methods attempt to reduce speckle noise using image processing techniques, which can use adaptive filters and/or wavelet analysis to process the acquired images. These methods can reduce the appearance of noise. Such methods often, however, do not recover information that is lost or buried in the speckle.

Thus, a need exists for improved methods and devices for reducing speckle noise in OCT imaging.

### SUMMARY

Apparatus, systems, and methods for optical coherence imaging are described herein. In some embodiments, an apparatus includes a light splitter and a detector. The light splitter receives a spatially coherent light beam and directs a first portion of the spatially coherent light beam to a reference arm and a second portion of the spatially coherent light beam to a sample arm. The sample arm includes a phase scrambler at least partially in a path of the second portion of the spatially coherent light beam. The phase scrambler is configured to produce a sample light beam having a spatially variable phase. The sample arm also includes a controller, operably coupled to the diffuser, to change the spatially variable phase of the sample light beam. The detector is in optical communication with the reference arm and the sample arm, and is configured to detect an interference pattern produced by interference of the first portion of the spatially coherent light beam propagated through the reference arm and a scattered beam produced by scattering of the sample light beam by a sample propagated through the sample arm.

In some embodiments, an apparatus includes an optical arm of an optical coherence tomography system, a lens, a phase scrambler and a controller. The optical arm defines at least a portion of a light path, and is configured to be in optical communication with a light source that produces a spatially coherent light beam propagating along the light path. The lens is within the light path of the optical arm. The phase scrambler is disposed at least partially within the light path, and is configured to produce, from the spatially coherent light beam, a scrambled light beam having a spatially variable phase. The controller is operably coupled to the phase scrambler, and is configured to change the spatially variable phase of the scrambled light beam.

In other embodiments, a method includes transmitting a first portion of a spatially coherent light beam through a reference arm and transmitting a second portion of the spatially coherent light beam through a sample arm. The transmitting of the second portion includes A) changing a local phase of the second portion of the spatially coherent light beam to produce a sample light beam and B) transmitting the sample light beam toward a sample. The method further includes detecting an interference pattern produced by interference of the first portion of the spatially coherent light beam propagated through the reference arm and a scattered portion of the sample light beam scattered by and/or reflected from the sample via the sample arm.

In yet other embodiments, a method of coherence tomography includes transmitting a light beam to illuminate a resolution volume associated with a sample. The light beam is spatially modulated to introduce a first local phase change to a first portion of the light beam and to introduce a second local phase change to a second portion of the light beam, the second local phase change different than the first local phase change. The light beam is temporally modulated to produce a first speckle pattern at a first time in a first image associated with the resolution volume and to produce a second speckle pattern at a second time in a second image associated with the resolution volume. The second speckle pattern is different than the first speckle pattern. The method further includes averaging the first speckle pattern with the second speckle pattern to reduce speckle noise in a third image associated with the resolution volume.

In yet other embodiments, an apparatus includes a light source to produce a spatially coherent light, a light splitter in optical communication with the light source to split the spatially coherent light into a first beam and a second beam, a scanner in optical communication with the beam splitter to scan the second beam across at least a portion of a sample at a first speed so as to scatter and/or reflect light from the sample, and a detector in optical communication with the light splitter to detect interference between the first beam and the light scattered and/or reflected from the sample. The apparatus also includes a phase scrambler disposed within a Rayleigh range of an image plane of a lens to diffuse the second beam. An image of the sample at the image plane has a first magnification with respect to the sample. The apparatus further includes an actuator configured to move the diffuser in a direction substantially orthogonal to an optical axis of the diffuser at a second speed no less than a product of the first magnification and the first speed.

### BRIEF DESCRIPTION OF THE DRAWINGS

FIG. 1 is a schematic illustration of an imaging system using time-varying phase change in the illumination light beams, according to an embodiment.

FIGS. 2A-2C is a schematic illustration showing the concept of speckle cancellation by temporally changing the local phase of a light beam in an imaging system.

FIG. 3 is a schematic illustration of an imaging system using time-varying phase change in the illumination light beams for ophthalmic applications, according to an embodiment.

FIG. 4 is a schematic illustration of an imaging system configured to change the phase of a light beam, the system including optical fibers, according to an embodiment.

FIG. 5 is a schematic illustration of an imaging system configured to change the phase of a light beam within a reference arm, according to an embodiment.

FIG. 6 is a schematic illustration of an imaging system configured to change the phase of a light beam by processing the beam in the Fourier domain, according to an embodiment.

FIG. 7 is a schematic illustration of an imaging system configured to change the phase of a light beam by processing the beam in the Fourier domain, according to an embodiment.

FIG. 8 is a schematic illustration of an imaging system configured to change the phase of a light beam by processing the beam in the Fourier domain within a sample arm, according to an embodiment.

FIGS. 9A and 9B are photographs of an example imaging system including a movable diffuser, according to an embodiment.

FIGS. 10 and 11 are schematic illustrations of endoscope imaging systems, according to an embodiment.

FIGS. 12A-12C show depth profiles of three diffusers that can be used in an imaging system to change the phase of a light beam.

FIG. 13 shows depth histograms of the diffusers shown in FIGS. 12A-12C.

FIG. 14 shows examples of effects of the diffusers shown in FIGS. 12A-12C on the optical power transmitted to the sample in coherence imaging.

FIG. 15 shows examples of effects of the diffusers shown in FIGS. 12A-12C on the signal levels in coherence imaging.

FIGS. 16A-16F illustrate examples of the effects of the diffusers shown in FIGS. 12A-12C on lateral resolution in coherence imaging.

FIG. 17 shows an example of a phantom structure forming a gap that can be used to study effective resolution of a speckle free optical coherence tomography (SFOCT) system according to an embodiment.

FIGS. 18A-18D show images acquired using standard OCT and SFOCT according to the embodiments described herein, of the gap shown in FIG. 17.

FIGS. 19A-19C shows example images of the gap shown in FIG. 17 obtained using standard OCT and SFOCT using a 2000 grit diffuser, SFOCT using a 1500 grit diffuser, respectively.

FIGS. 20A-20C show example images obtained using standard OCT and SFOCT according to the embodiments described herein, respectively, along with the lines that represents the segmentation boundary between the phantom structure and the gap shown in FIG. 17.

FIG. 21 shows an example of a compilation of registration of the segmentation boundaries with an image of the phantom structure shown in FIG. 17 taken with a bright-field microscope.

FIG. 22A is a graph showing the size of the gap shown in FIG. 17 as a function of location measured by different methods, including standard OCT and SFOCT using a 2000 grit diffuser, SFOCT using a 1500 grit diffuser, and microscope.

FIG. 22B is a graph showing the size of the gap in the standard OCT and SFOCT images (as shown in FIG. 22A) plotted as a function of the size of the gap measured in the microscope image.

FIGS. 23A-23B are simulation results of pixel value statistics of images acquired by SFOCT systems according to an embodiment.

FIGS. 24A-24C are example images of phantoms made of gold nano-rods (GNR)s dispersed in agarose, acquired using standard OCT, SFOCT with 2000 grit diffuser, and SFOCT with 1500 grit diffuser, respectively.

FIGS. 25A-25B are graphs showing example statistical analysis of pixel values of scans of a GNR phantom obtained with standard OCT and SFOCT, respectively.

FIGS. 25C-25D show example reduction in normalized STD versus the number of averages for OCT and SFOCT, respectively.

FIGS. 26A-26C are graphs showing example statistical analysis of pixel values of images taken by SFOCT using a 2000 grit diffuser.

FIGS. 27A-27K show images taken using standard OCT and SFOCT, according to an embodiment, of a thin slice of a GNR-agarose phantom with 3 μm diameter beads.

FIGS. 27L-27M are graphs of the normalized intensity for standard OCT and SFOCT, according to an embodiment, of the thin slice of a GNR-agarose phantom with 3 μm diameter beads.

FIGS. 28A-28I show images taken using standard OCT and SFOCT, according to an embodiment, of biological samples.

FIGS. 29A-29I show images taken using standard OCT and SFOCT, according to an embodiment, of mouse cornea and retina.

FIGS. 30A-30D show images taken using standard OCT and SFOCT, according to an embodiment, of a human retina.

FIGS. 31A-31G show images taken using standard OCT and SFOCT, according to an embodiment, of finger tips.

FIGS. 32A-32B show images taken using standard OCT and SFOCT, according to an embodiment, of finger tips with sweat ducts.

FIGS. 33A-33H are images showing speckle noise processing using SFOCT, spatial compounding, and 3D smoothing techniques.

FIGS. 34A-34J are images showing speckle noise processing using SFOCT and digital filtering methods.

FIGS. 35A-35B show an image and a spectral analysis image, respectively, of a tumor in an ear pinna of a mouse based on scans obtained with standard OCT.

FIGS. 35C-35DB show an image and a spectral analysis image, respectively, based on scans obtained with SFOCT according to an embodiment.

FIGS. 36A-36B show images of a fingertip taken using convention OCT methods and SFOCT methods according to an embodiment, respectively.

FIGS. 36C-36D are graphs of the pixel values (intensity) as a function of depth from the images shown in FIGS. 36A and 36B, respectively.

FIGS. 36E-36F are plots showing the calculated exponential coefficients and the associated confidence bounds from the images shown in FIGS. 36A and 36B, respectively.

FIG. 37A is an of a mouse retina taken using SFOCT methods according to an embodiment.

FIGS. 37B-37D flattened images of the region of interested identified in FIG. 37A for a corresponding image taken using convention OCT, a corresponding image taken using convention OCT with lateral smoothing applied, and the SFOCT image, respectively.

FIG. 38 is a flow chart of a method of optical coherence tomography according to an embodiment.

FIG. 39 is a flow chart of a method of optical coherence tomography according to an embodiment.

### DETAILED DESCRIPTION

Apparatus, systems, and methods for optical coherence imaging are described herein. In some embodiments, an apparatus includes a light splitter and a detector. The light splitter receives a spatially coherent light beam and directs a first portion of the spatially coherent light beam to a reference arm and a second portion of the spatially coherent light beam to a sample arm. The sample arm includes a phase scrambler at least partially in a path of the second portion of the spatially coherent light beam. The phase scrambler is configured to produce a sample light beam having a spatially variable phase. The sample arm also includes a controller, operably coupled to the diffuser, to change the spatially variable phase of the sample light beam. The detector is in optical communication with the reference arm and the sample arm, and is configured to detect an interference pattern produced by interference of the first portion of the spatially coherent light beam propagated through the reference arm and a scattered beam produced by scattering of the sample light beam by a sample propagated through the sample arm.

In some embodiments, an apparatus includes an optical arm of an optical coherence tomography system, a lens, a phase scrambler and a controller. The optical arm defines at least a portion of a light path, and is configured to be in optical communication with a light source that produces a spatially coherent light beam propagating along the light path. The lens is within the light path of the optical arm. The phase scrambler is disposed at least partially within the light path, and is configured to produce, from the spatially coherent light beam, a scrambled light beam having a spatially variable phase. The controller is operably coupled to the phase scrambler, and is configured to change the spatially variable phase of the scrambled light beam.

In some embodiments, an apparatus includes a light source to produce a spatially coherent light, a light splitter, a scanner, a detector a phase scrambler and an actuator. The light splitter is in optical communication with the light source, and splits the spatially coherent light into a first beam and a second beam. The scanner is in optical communication with the light splitter, and is configured to scan the second beam across at least a portion of a sample at a first speed to produce a scattered beam scattered by the sample. The detector is in optical communication with the light splitter, and is configured to detect an interference between the first beam and the scattered beam. The phase scrambler is disposed within a Rayleigh range of an image plane of a lens, and is configured to modulate a local phase of the second beam. An image of the sample at the image plane has a first magnification with respect to the sample. The actuator is configured to move the phase scrambler in a direction substantially orthogonal to an optical axis of the phase scrambler at a second speed no less than a product of the first magnification and the first speed.

In some embodiments, a method of coherence tomography includes transmitting a light beam to illuminate a resolution volume associated with a sample. The light beam is spatially modulated to introduce a first local phase change to a first portion of the light beam and to introduce a second local phase change to a second portion of the light beam. The second local phase change is different than the first local phase change. The light beam is temporally modulated to produce a first speckle pattern at a first time in a first image associated with the resolution volume and to produce a second speckle pattern at a second time in a second image associated with the resolution volume. The second speckle pattern is different than the first speckle pattern. The method further includes averaging the first speckle pattern with the second speckle pattern to reduce speckle noise in a third image associated with the resolution volume.

In other embodiments, a method includes transmitting a first portion of a spatially coherent light beam through a reference arm and transmitting a second portion of the spatially coherent light beam through a sample arm. The transmitting of the second portion includes A) changing a local phase of the second portion of the spatially coherent light beam to produce a sample light beam and B) transmitting the sample light beam toward a sample. The method further includes detecting an interference pattern produced by interference of the first portion of the spatially coherent light beam propagated through the reference arm and a scattered portion of the sample light beam scattered by and/or reflected from the sample via the sample arm.

In yet other embodiments, a method of coherence tomography includes transmitting from a light source a light beam to a resolution volume associated with a sample. A first interference pattern is detected, at a first time and when the light beam is at a beam position relative to the sample. The first interference pattern is associated with the resolution volume, and is produced, in part, by a first scattered beam produced by scattering of the light beam from the resolution volume. The method includes changing a local phase of the light beam within the resolution volume of the sample. A second interference pattern is detected, at a second time after the changing and when the light beam is at the beam position relative to the sample. The second interference pattern is associated with the resolution volume, and is produced, in part, by a second scattered beam produced by scattering of the light beam having the changed local phase from the resolution volume. The first interference pattern and the second interference pattern are averaged.

In yet other embodiments, a method of coherence tomography includes transmitting from a light source a reference beam portion of a spatially coherent light beam to a reference member. The method includes, transmitting from the light source a sample beam portion of the spatially coherent light beam to a resolution volume associated with a sample. A local phase of at least one of the reference beam portion or the sample beam portion is changed. A first interference pattern is detected, at a first time and when the sample beam portion is in a beam position relative to the sample. The first interference pattern is associated with the resolution volume, and is produced based on the reference beam portion and the sample beam portion. The method includes changing, at a second time after the first time, the local phase of at least one of the reference beam portion or the sample beam portion. A second interference pattern is detected, at a third time and when the sample beam portion is in the beam position. The second interference pattern is associated with the resolution volume, and is produced based on the reference beam portion and the sample beam portion. The first interference pattern and the second interference pattern are averaged.

In yet other embodiments, an apparatus includes an elongated member, an optical transmission member, a lens and a phase scrambler. The elongated member is configured to be disposed within a bodily cavity, and defines a lumen. A side wall of the elongated member defines an opening. The optical transmission member is disposed within the lumen, and is configured to convey a sample light beam therethrough. The sample light beam is spatially coherent within the optical transmission member. The lens is disposed within the lumen and is optically coupled to the optical transmission member. The lens, the optical transmission member, and the opening of the elongated member define at least a portion of a sample light path through which the sample light beam is conveyed to a sample. The phase scrambler is disposed at least partially within a sample light path. The phase scrambler is configured to change a local phase of the spatially coherent sample light beam conveyed from the optical transmission member.

The term “about” when used in connection with a referenced numeric indication means the referenced numeric indication plus or minus up to 10 percent of that referenced numeric indication. For example, “about 100” means from 90 to 110.

In a similar manner, term “substantially” or “approximately” when used in connection with, for example, a geometric relationship, a numerical value, and/or a range is intended to convey that the geometric relationship (or the structures described thereby), the number, and/or the range so defined is nominally the recited geometric relationship, number, and/or range. For example, two structures described herein as being “substantially parallel” is intended to convey that, although a parallel geometric relationship is desirable, some non-parallelism can occur in a “substantially parallel” arrangement. By way of another example, a structured placed “approximately within an image plane” is intended to convey that, while the recited position is desirable, some tolerances can occur. Such tolerances can result from imperfections in optics that define the image plane, e.g., manufacturing tolerances, measurement tolerances, and/or other practical considerations. As described above, a suitable tolerance can be, for example, of ±10% of the stated geometric construction, numerical value, and/or range.

As used in this specification and the appended claims, the words “proximal” and “distal” refer to direction closer to and away from, respectively, an operator of the device. Thus, for example, the end of an imaging device adjacent or contacting the patient's body would be the distal end of the imaging device, while the end opposite the distal end would be the proximal end of the imaging device.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

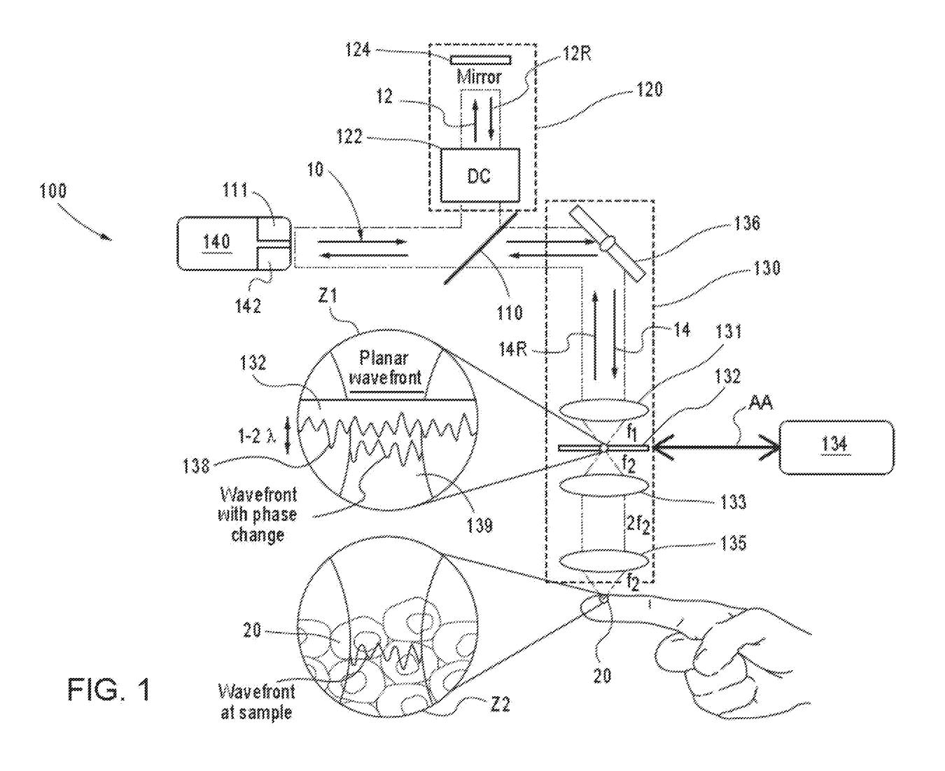

FIG. 1 shows a schematic of a system 100 that can reduce or eliminate speckle noise in OCT without compromising the resolution of the imaging, according to an embodiment. The system 100 and any of the optical systems disclosed herein can generally be referred to as a “speckle-free optical coherence tomography” (SFOCT) system. In general, the system induces a change in the speckle pattern by introducing local phase shifts within the light beam illuminating the sample. Said another way, the system 100 transforms a spatially coherent light beam into a sample light beam that has a spatially variable phase. Varying the local phase of the sample light beam in time (i.e., between each image capture event) can create images with non-correlated speckle patterns that can be compounded to create an image with reduced speckle noise. The system 100, and any of the other systems and methods disclosed herein, can therefore reduce the actual speckle noise, instead of the appearance of speckle noise, as is accomplished with image processing. Therefore, the systems and methods described herein, including the system 100, can clarify and reveal structures that are otherwise undetectable in OCT images due to speckle noise. Said another way, the systems and methods described herein, including the system 100, can recover information buried in the speckle noise.

The system 100 includes a light assembly 140, a beam splitter 110 (also generally referred to as a light splitter), a reference arm 120, and a sample arm 130. The light assembly 140 includes a light source 111 and a detector 142. The light source 111 can be any suitable light source of the types shown and described herein that produces a spatially coherent light beam 10 of a desired wavelength (or average wavelength, in instances where the light beam is a broadband beam). For example, in some embodiments, the light source 111 (and any of the light sources described herein) can be a super-luminescent diode (SLED or SLD). A SLD normally operates like edge-emitting laser diodes (EELD) but without optical feedback or a cavity. Super-luminescence may occur when the spontaneous emission experiences gain due to higher injection currents. The higher gain can cause a superlinear power increase and an increasing narrowing of the spectral width. The radiation emitted by an SLD can be amplified spontaneous emission (ASE) and have a low time-coherence. Since SLDs are generally implemented in wave-guide structures, the space-coherence of the emitted radiation can be accordingly high. The wavelength is determined by the material and its layering within the diode semiconductor. Practical wavelengths of SLD includes 675 nm, 820 nm, 930 nm, 1300 nm, 1550 nm, or any other wavelength known in the art.

In other embodiments, the light source 111 (and any of the light sources described herein) can include one or more of the following: a Ti:Sapphire laser at 800 nm, a Cr:forsterite laser at 1280 nm, a LED at 1240 nm-1300 nm, an amplified spontaneous emission (ASE) fiber source at 1300 nm-1550 nm, a super-fluorescence source such as a Yb-doped fiber (1064 nm), an Er-doped fiber (1550 nm), and a Tm-doped fober (1800 nm), a photonic crystal fiber at 725 nm or 1300 nm, or a thermal tungsten halogen source at 880 nm. The wavelength (or average wavelength) used in the SFOCT can be dependent on, for example, the desired penetration depth of the light in the sample.

The detector 142 and any of the detectors described herein can be any suitable detector that detects and/or receives light scattered and/or reflected from the sample and any reference elements. The detector 142 and any of the detectors described herein can include, for example, a charge coupled device (CCD). The detector 142 and any of the detectors described herein can also be referred to as a spectrometer. As described herein, the returned first portion 12R and returned second portion 14R of the light beam 10 are combined at the detector 142, which detects an interference pattern (when the system is properly aligned) produced by interference of the first return portion 12R and the second return portion 14R. An image of a sample 20 can then be extracted from the interference pattern detected by the detector 142.

The light splitter 110 receives a spatially coherent light beam 10 and splits the spatially coherent light beam into a first portion 12 and a second portion 14. The first portion 12 enters a reference arm 120, which further includes a dispersion compensation element 122 and a reference arm mirror 124. The dispersion compensation element 122 can compensate for dispersion introduced in the sample arm 130 to match the dispersion between the first portion of the light 12 and the second portion light 14 when they are returned to the detector 142 and combined to produce interferences. The reference arm mirror 124 can reflect the first portion 12 to propagate a first return portion 12R back to the light splitter 110. The light splitter the further reflects the first return portion 12R to a detector 142.

The second portion of the light 14 enters the sample arm 130. The sample arm 130 is configured to interact with a sample 20, such as a bodily tissue, to allow for optical coherence imaging of a portion of the sample 20. The sample arm 130 includes a phase scrambler 132 (also generally referred to as a diffuser or a local phase randomizer), a controller 134, and a series of lenses and/or optical components, as described below. Specifically, as shown in FIG. 1, the sample arm 130 includes a galvo mirror 136, which can direct the second portion 14 of the spatially coherent beam 10 toward different directions. In the manner the second portion 14 of the light beam can be scanned (or traversed) across the surface of the sample 20. In some examples, a Micro-electromechanical system (MEMS) scanner can be employed to control the mirror 136.

The sample arm 130 also includes a first lens 131 having a first focal length f₁, a second lens 133 having a second focal length f₂, and a third lens 133 also having a second focal length f₂. These three lens 131, 133, and 135 form a 4f configuration: the distance between the first lens 131 and the second lens 133 is f₁+f₂, the distance between the second lens 133 and the third lens 135 is 2×f₂, and the distance between the third lens 135 and the sample 20 is f₂. The 4f configuration can help relay the image of the sample 20 back to the detector 142.

The phase scrambler 132 is disposed at least partially in a light path 139 of the second portion 14 of the spatially coherent light beam 10. In this example, the phase scrambler 132 is a substantially transparent object (transparent to the wavelength of the light beam 10 used in the system 100), and includes a surface 138 (see the zoomed region Z1 of FIG. 1) that has random or nearly random distribution of micro diffusion centers (e.g., a rough surface). In this manner, the phase scrambler 132 can reduce a spatial coherence of the second portion 14 of the spatially coherent light beam 10. More particularly, as shown in the zoomed region Z1 of FIG. 1, the second portion 14 of the spatially coherent light beam 10 has a planar wavefront as it approaches the phase scrambler 132. As the second portion 14 passes through the phase scrambler 132, the phase scrambler 132 produces a sample light beam having a spatially variable phase. This is indicated by the “downstream” wavefront, which is shown as having a local variation in the phase. In some embodiments, the phase scrambler 132 (and any of the phase scramblers and/or diffusers described herein) can include, but are not limited to, a ground glass element, a sandblasted glass element, an opal diffusing glass, or a holographic optical element.

The sample light beam, with the spatially variable phase, is then directed towards the sample 20, as shown in the zoomed region Z2 of FIG. 1. The sample 20 reflects, diffuses, and/or scatters at least part of the sample light beam, and the reflected, diffused, or scattered part normally propagates along the same beam path as that of the incident beam (i.e., the beam path travelled by the second portion 14) and reaches the detector 142. The returned beam from the sample is identified in FIG. 1 as the second return portion 14R.

The controller 134 is operably coupled to the phase scrambler 132 to change the spatially variable phase of the second portion 14 of the spatially coherent light beam 10 as it passes through the phase scrambler 132. Similarly stated, the controller is operably coupled to the phase scrambler 132 and temporally changes the spatially variable phase of the sample light beam (i.e., the light beam that is propagated to the sample 20). The controller can be any suitable controller and/or can include any suitable mechanism to temporally change the phase of the sample light beam. For example, in some embodiments, the controller 134 includes an actuator to move the phase scrambler 132 within the path 139 of the spatially coherent light beam 10 in the phase scrambler 132. By moving the phase scrambler 132, the random distribution of micro diffusion centers on the surface 138 are moved, and thus the spatial variation of phase in the sample light beam is changed.

In some embodiments, the phase scrambler 132 is disposed approximately at an image plane of a lens (e.g., the lens 131 or the lens 133) within the sample arm 130. Similarly stated, in some embodiments, the phase scrambler 132 is aligned with a focal plane associated with the sample 20 and/or a lens (e.g., the lens 131 or the lens 133) within the sample arm 130. In some embodiments, the phase scrambler 132 is disposed within a Rayleigh range of the image plane associated with the sample 20 and/or a lens (e.g., the lens 131 or the lens 133) within the sample arm 130. In this manner, the phase scrambler 132 acts to change the local phase of the sample light beam, as described above. In some such embodiments, the controller 134 includes an actuator (not shown) to move the phase scrambler within the image plane, as shown by the arrow AA in FIG. 1. In some such embodiments, the controller 134 includes an actuator (not shown) to move the phase scrambler along a direction non-parallel (e.g., substantially perpendicular to) a propagation direction of the second portion of the spatially coherent light beam 14, as shown by the arrow AA in FIG. 1.

In some embodiments, the controller 134 includes an actuator (not shown) to rotate the phase scrambler within the image plane. The actuator can be, for example, a motor that rotates the phase scrambler 132 continuously during a sampling operation.

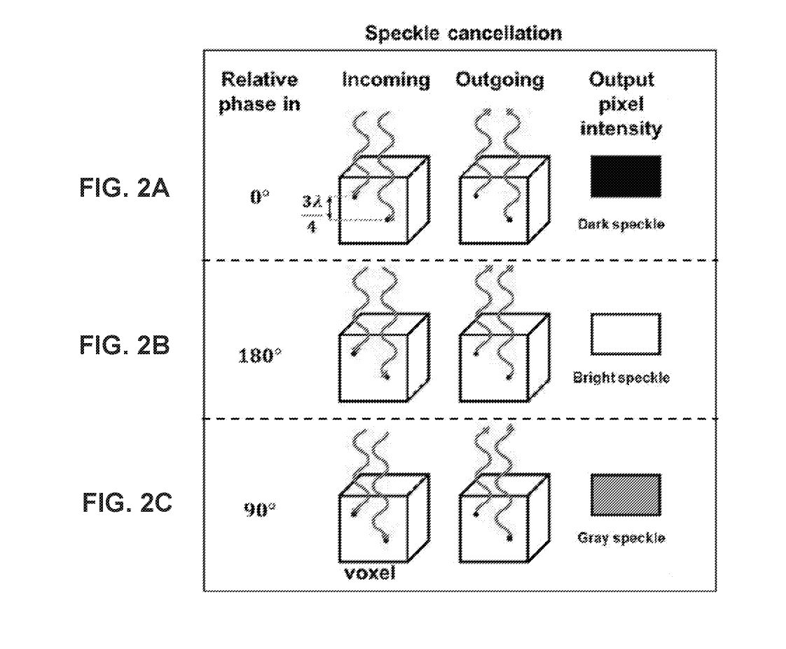

In some examples, the phase scrambler 132 and the controller 134 introduce spatial modulation into the light beam over the size of an imaging voxel (or a resolution volume of the sample 20). In some examples, the phase scrambler 132 and the controller 134 introduce temporal modulated over the course of A-scan acquisition (e.g., microseconds or longer). For example, FIGS. 2A-2C illustrate speckle cancellation using time-varying diffusion, which can be introduced by time-varying phase shift between neighboring scatters. FIG. 2A shows a first moment at which two incident light waves have zero phase shift. The two incident light waves illuminate two scatterers (black dots) separated by 3λ/4 within a resolution volume (the cubic) on a sample to be imaged (e.g., the sample 20). Upon being scattered by the two scatterers, the two outgoing light waves have a 180° phase shift and destructively interfere with each other on the detector, creating a dark speckle. FIG. 2B shows a second moment at which two incident light waves have 180° phase shift. Upon scattering, the two outgoing light waves have a zero phase shift and constructively interfere with each other on the detector, creating a bright speckle. FIG. 2C shows a third moment at which two incident light waves have 90° phase shift. Upon scattering, the two outgoing light waves also have a 90° phase shift and the resulting speckle is between dark and bright (a gray speckle). Therefore, by introducing different phase shifts between neighboring incident waves within a resolution cells, different speckle patterns can be created and can be cancelling each other when they are averaged.

In use, the controller 134 and the detector 142 can be coordinated such that each image (or the original interference pattern) taken by the detector 142 includes a different speckle noise pattern created by a different diffusion introduced by the phase scrambler 132. In some examples, this can be achieved by tuning image taking rate of the detector 142 to be greater than the diffusion changing rate (or phase scrambling rate) of the controller 134 for the spatial light modulator. As described above, in some embodiments, the phase scrambler 132 has a static diffusion property (e.g., a ground glass), and the controller 134 can be configured to move the phase scrambler 132 by a substantial distance within the time interval of successive images taken by the detector 142. In these examples, the substantial distance can be, for example, comparable to the size of the second portion 14 of the spatially coherent light beam 10 (e.g., more than half of the diameter, more than a quarter of the diameter, more than a tenth of the diameter, etc.). In other embodiments, the distance moved by the phase scrambler 132 between successive images can be related to the wavelength (or average wavelength) of the light beam 10. For example, in some embodiments, the distance moved by the phase scrambler 132 between successive images can be about one wavelength (or average wavelength). In other embodiments, the distance moved by the phase scrambler 132 between successive images can be about two wavelengths (or two times the average wavelength).

In some embodiments, it is desirable to minimize movement when an image is being captured (to avoid blurring of the image), but maximize movement between the taking of images. In some embodiments, however, the phase scrambler 132 can be moved continuously during a sampling event (i.e., both during and between successive images). In some such embodiments, the phase scrambler 132 can be moved continuously during a sampling event at a speed of less than about one wavelength (or average wavelength). In some such embodiments, the phase scrambler 132 can be moved continuously during a sampling event at a speed of about one-third of a wavelength (or average wavelength).

The coordination between the controller 134 and the detector 142 can be carried out using any suitable software. For example, in some embodiments, the controller 134 can use software, such as Thorlabs APT (ThorLabs, Newton, N.J.).

In use, to reduce speckle noise in the images produced by the interference patterns, the system 100 can be operated according to an illustrative and non-limiting method below, as well as any other methods described herein. The first portion 12 of the spatially coherent light beam 10 is transmitted through the reference arm 120, and the second portion 14 of the spatially coherent light beam 10 is transmitted through the sample arm 130. In the sample arm 130, a time-varying local phase change is introduced into the second portion 14 of the spatially coherent light beam 10 by time-varyingly changing the diffusion produced by the phase scrambler 132. As described above, this can include moving the phase scrambler 132 along a direction perpendicular to the propagation direction of the second portion 14. After the phase scrambler 132, the sample light portion (shown with a non-planar wavefront) is then transmitted to the sample 20, where at least part of the sample light portion is reflected, diffused, and/or scattered back to the sample arm 130, which then transmits the reflected, diffused, or scattered part 14R to the detector 142. The detector 142 combines the second returned portion 14R with the first return portion 12R to produce an interference pattern. Multiple interference patterns can be taken by the detector 142. Each interference pattern is taken at a different timing moment. Due to the time-varying phase change introduced into the second portion 14 pf the light, the resulting interferences patterns taken at different timing moments include different and uncorrelated speckle noise patterns created by the diffusion. An image can be extracted from each interference pattern and the ultimate image of the sample can be produced by averaging the multiple images extracted from the multiple interference patterns.

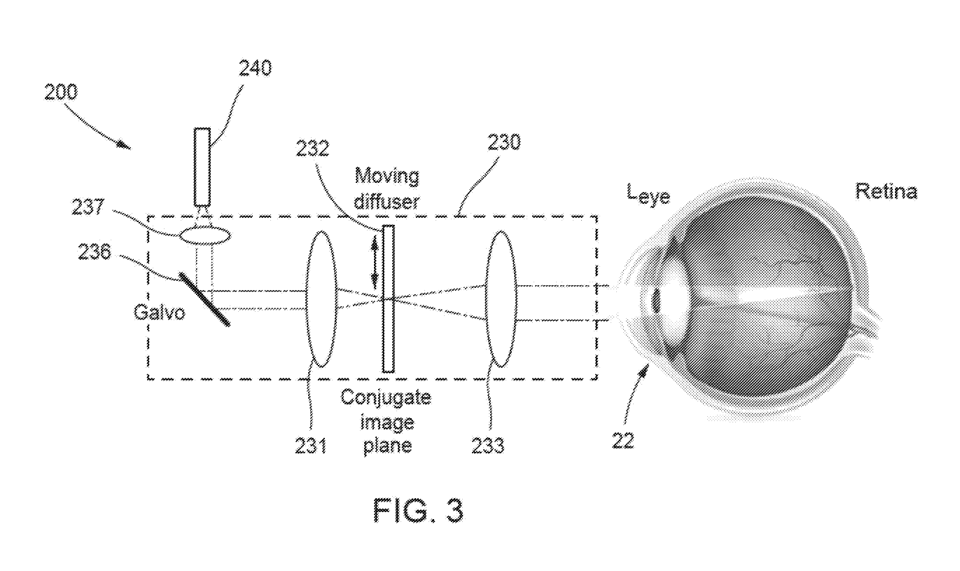

Although the system 100 is shown and described as including a particular lens configuration (i.e., the 4f configuration), in other embodiments, any suitable lens configuration can be used in conjunction with a phase scrambler 132 (or any other phase scramblers shown herein) and to produce images according to any of the methods described herein. For example, FIG. 3 is a schematic view of a system, according to an embodiment, that can be used for ophthalmic purposes, such as diagnostics of eye diseases. The system 200 includes a reference arm (not shown) and a sample arm 230. In the sample arm, light beams propagate through a collecting lens 237, a galvo mirror 236, a first lens 231, a phase scrambler 232, and a second lens 233 before reaching a sample eye 22. Compared to the system 100 shown in FIG. 1, the system 200 does not employ the 4f configuration to replicate the image plane (i.e., no need to use the third lens 135 shown in FIG. 1), at least because the eye 22 normally includes a natural lens.

The phase scrambler 232 can be similar to the phase scrambler 132 shown and described above, and can located in any suitable position within the sample arm 230. For example, in some embodiments, the phase scrambler 232 can be located within an image plane, and can be configured to move (e.g., by a controller, not shown). In other embodiments, however, the phase scrambler 232 can be a stationary phase scrambler, of any type shown and described herein.

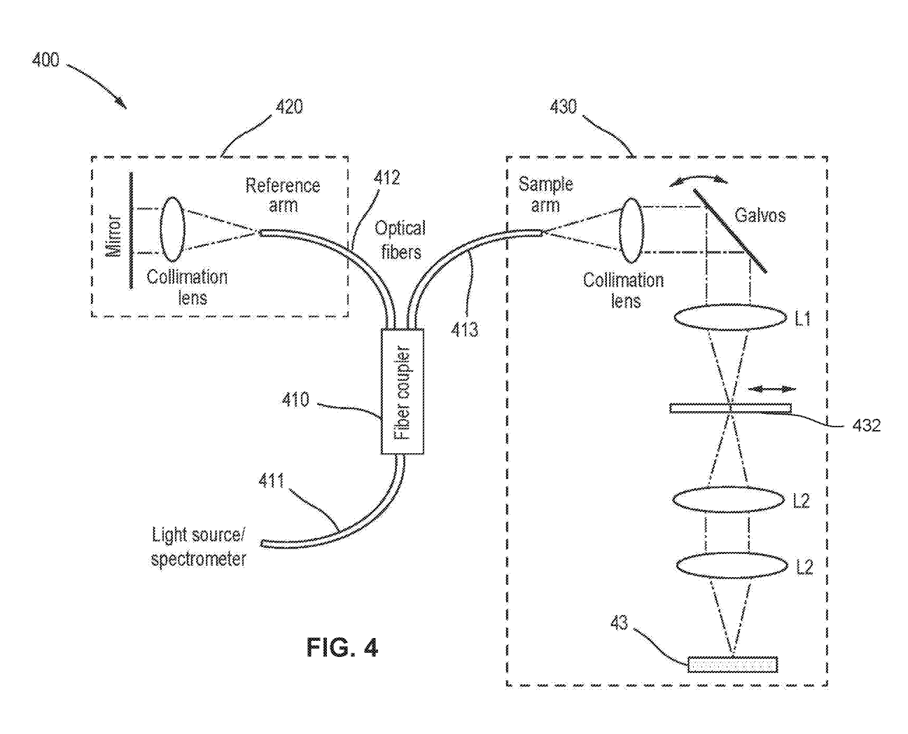

In some embodiments, a system can include any suitable structure and/or optical components to define the light paths through which the beams of light can be propagated. For example, FIG. 4 is a schematic illustration of an imaging system in which optical fibers can be used to propagate light beams from one component of the system to another. More specifically, the system 400 includes a fiber coupler 410 to receive a light beam from a light source fiber 411 (e.g., a fiber in optically communication with a light source) and split the received light beam into a first portion and a second portion. The first portion goes to a reference arm 420 via a first fiber 412 and the second portion goes to a sample arm 430 via a second fiber 413. The first portion of light, when reflected from the reference arm 420, can propagate back to the detector (not shown) through the same beam path along the first fiber 412, the fiber coupler 410, and the light source fiber 411. Similarly, the second portion of light, when scattered and/or reflected by the sample 43, can propagate back to the detector through the same beam path along the first fiber 413, the fiber coupler 410, and the light source fiber 411 to combine with the first portion and generate interference patterns. The reference arm 420 can be substantially the same as any of the reference arms described herein, including the reference arms 120 and 320. Similarly, the sample arm 430 can be substantially the same as the sample arm 130 and 330 described before, or any other sample arm disclosed in this application. Specifically, the sample arm 430 can include a phase scrambler 432, which can be similar to the phase scrambler 132 shown and described above, and can located in any suitable position within the sample arm 430. For example, in some embodiments, the phase scrambler 432 can be located within an image plane, and can be configured to move (e.g., by a controller, not shown). In other embodiments, however, the phase scrambler 432 can be a stationary phase scrambler, of any type shown and described herein.

In other embodiments, depending on the operating wavelength of the imaging systems, other transmission devices, such as waveguides, can be used to transmit light or other radiation within the system.

Although the system 100 is shown and described as including a phase scrambler 132 within the sample arm 130, in other embodiments, an imaging system using time-varying phase scrambling can be implemented by placing a phase scrambler and/or a diffuser in the reference arm. This implementation can result in less aberration induced by the phase scrambler and/or the diffuser without reducing the optical power on the sample. This implementation includes focusing the light on the reference arm on to a phase scrambler, such as a moving diffuser, a rough mirror, or a spatial light modulator.

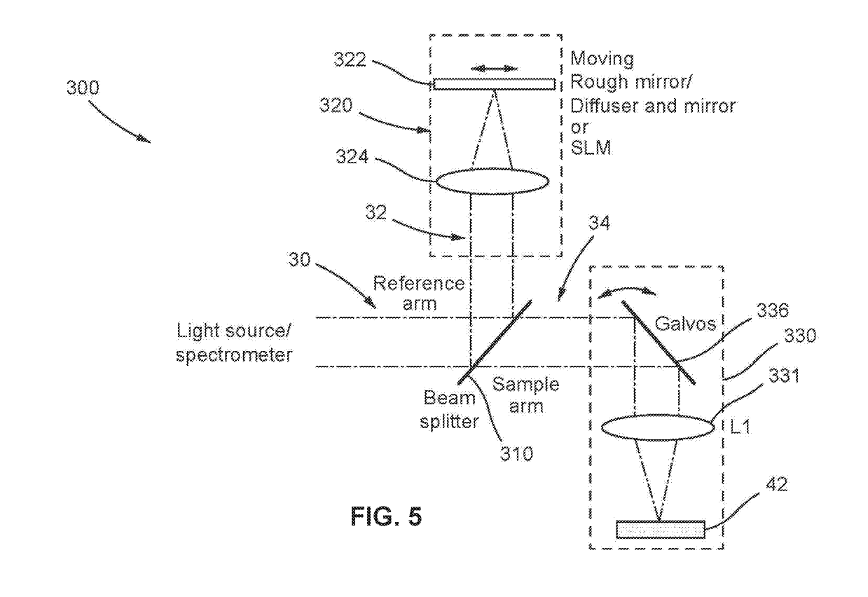

For example, FIG. 5 is a schematic of an imaging system 300, in which the time-varying diffusion is introduced in the reference arm. More specifically, the system 300 includes a beam splitter 310 to receive a spatially coherent light beam 30 and split the spatially coherent light beam 30 into a first portion 32 and a second portion 34. The first portion 32 enters a reference arm 320, which further includes a reference arm lens 324 and a phase scrambler (also referred to as a diffuser) 322 that is placed within or close to the focal plane of the reference arm lens 324. The phase scrambler 322 can reflect, scatter, or diffuse at least part of the first portion 32 of the spatially coherent light beam 30 back to a detector (not shown) for interference generation. The second portion 34 enters a sample arm 330, which includes a galvo mirror 336 to scan (or move) the second portion 34 of the spatially coherent light beam 30 across a sample 42 to be imaged. The sample arm 330 also includes an imaging lens 331 to collect light scattered, reflected, and/or diffused by the sample 42 and to transmit the collected light back to the detector (not shown) to interfere with the first portion 32 of the spatially coherent light beam 30. The returned first portion 32 and the returned second portion 34 of the spatially coherent light beam 30 are combined at the detector (not shown), which detects an interference pattern produced by interference of the returned first portion 32 and the returned second portion 34. An image of the sample 42 can then be extracted from the interference pattern detected by the detector. In FIG. 5, the phase scrambler 322 is used to create the time-varying change in the local phase to implement any of the methods described herein.

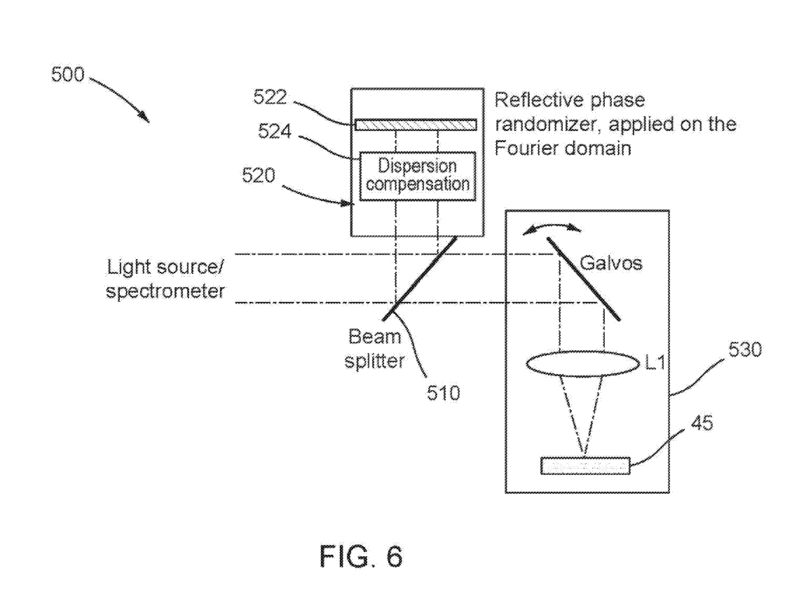

Although the optical systems described above, including the system 100, are shown and described as including a phase scrambler that is disposed within an image plane of a focused beam, in other embodiments, a system can include a phase scrambler disposed at any suitable location within a light path. Moreover, in some embodiments, systems and methods can include producing a local change of phase in a collimated (and not a focused) beam. Specifically, in some embodiments, a system can be configured employ phase scrambling in the Fourier domain. For example, FIG. 6 is a schematic of an imaging system 500 in which changing of the local phase is achieved by processing the light beam on the Fourier transform of the image plane. This can be implemented by applying local, position dependent, time-varying phase-amplitude when the beam is collimated. This phase scrambling can be applied either on the sample arm or on the reference arm. Since scrambling is in the Fourier domain, amplitude scrambling (e.g., by blocking or attenuating regions in the Fourier domain) and phase scrambling (e.g., by changing the path length of the light locally using a glass diffuser or a spatial light modulator) can result in phase scrambling in the spatial domain. Therefore, the effect can be similar to examples described herein, for example, with the system 100.

The imaging system 500 shown in FIG. 6 includes a beam splitter 510 to receive a light beam and divide the beam into a first portion and a second portion. The first portion is then transmitted into a reference arm 520, which further includes a dispersion compensation element 524 and a phase scrambler 522 applied on the Fourier domain when the first portion of the light beam is collimated (i.e., not focused). The phase scrambler 522 in this example is a reflective phase randomizer, i.e., phase randomization is applied when a light beam is reflected back by the phase scrambler 522 (also referred to as an optical element in the Fourier domain). The second portion is transmitted into a sample arm 530 to illuminate at least a portion of a sample 45 to be imaged. The sample arm 530 can be substantially similar to any of the example sample arms disclosed in this application.

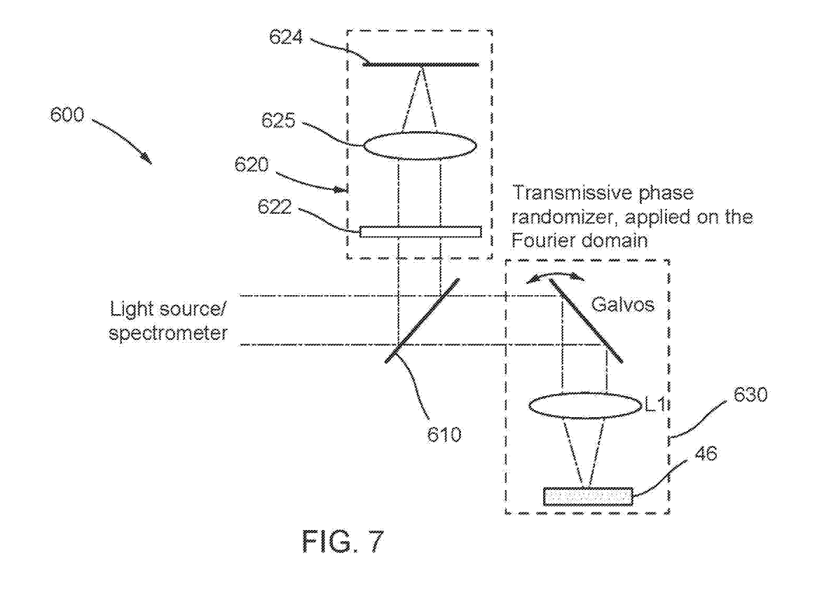

FIG. 7 shows another example imaging system 600, which includes phase scrambling in the reference arm. More specifically, the imaging system 600 includes a beam splitter 610 to receive a light beam and divide the beam into a first portion and a second portion. The first portion is then transmitted into a reference arm 620, which further includes a phase scrambler 622 applied on the Fourier domain when the first portion of the light beam is collimated (i.e., not focused), a reference arm lens 625, and a reflector 624 disposed approximately at the focal plane of the reference arm lens 625. The phase scrambler 622 (also referred to as an optical element in the Fourier domain) in this example is a transmissive phase randomizer, i.e. phase randomization is applied when a light beam is transmitted through the phase scrambler 622. The second portion is transmitted into a sample arm 630 to illuminate at least a portion of a sample 46 to be imaged. The sample arm 630 can be substantially similar to any of the example sample arms disclosed in this application.

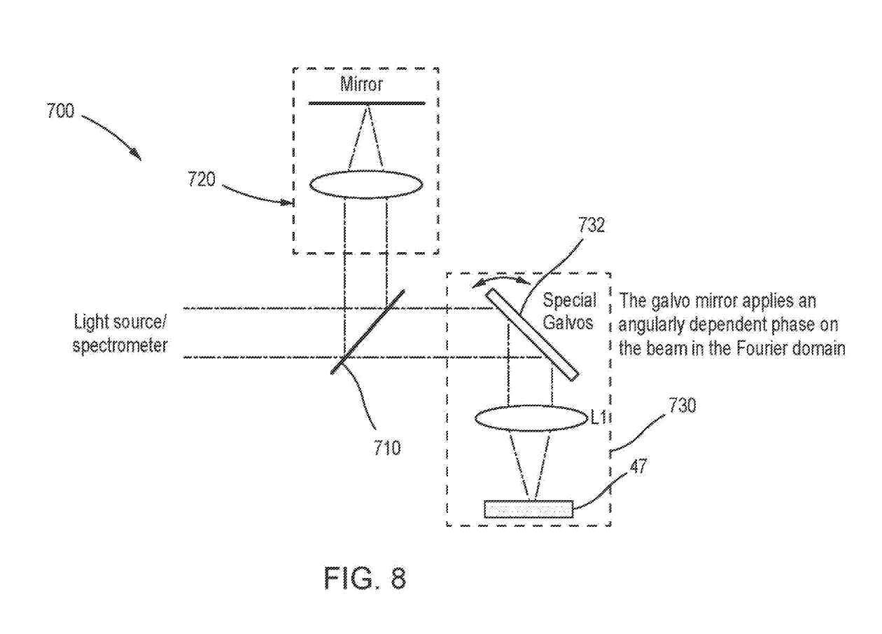

FIG. 8 shows a schematic of an imaging system 700 which applies angularly dependent phase and/or amplitude variation to the light beam for imaging and speckle noise reduction. In general, applying phase (and/or amplitude) randomization (or scrambling) on the Fourier transform of the image plane can be achieved by applying angularly dependent phase when the beam is collimated rather than focused. Owing to the Fourier relationship, applying an angle dependent phase in the Fourier domain can be equivalent to applying a position dependent phase in the spatial domain.

For example, the imaging system 700 shown in FIG. 8 includes a beam splitter 710 to receive a light beam and divided the beam into a first portion and a second portion. The first portion enters a reference arm 720, which can be substantially similar to any of the reference arms disclosed in this application. The second portion enters a sample arm 730, which further includes a galvo mirror 732 to, among other things, scan (or move) the second portion across a sample 47 to be imaged. In addition, the galvo mirror 732 also introduces an angle dependent phase variation into the second portion of the light beam. In other words, the phase scrambling can be a function of ray propagation angle. Therefore, the optical path lengths (OPL) between rays having different propagating angles are randomized. Without being bound by any particular theory or mode of operation, an OPL can be written as OPL=∫(n*dz) where n is the index of refraction and dz is a differential length element along the path of the ray. Therefore, an angle dependent phase variation can be introduced by using a material which has a refractive index changing as a function of propagation angle, i.e., an anisotropic material. Example materials include, but are not limited to, a biaxial or uniaxial material like calcite or a liquid crystal array where the permeability is a tensor. Photonic crystal devices and other structured materials can also have different refractive indices as a function of propagation direction and can also be used. Furthermore, the phase variation introduced by the galvo mirror 732 can also depend on the polarization of light beam, thereby adding a second degree of randomization and further improving the speckle reduction.

In some embodiments, a Speckle Free Optical Coherence Tomography (SFOCT) system, such as the systems 100 and 200 shown above (or any other systems shown and described herein), can be constructed from commercially available OCT systems. For example, in some embodiments, a kit can include a phase scrambler of the types shown and described herein, a controller, and the necessary hardware to mount the phase scrambler and hardware within a commercial OCT system. In one example, a SFOCT system is built based on the Ganymede HR system manufactured by Thorlabs Inc. In this example, a diffuser can be placed at the focal plane of the original OCT probe and a new image plane can be projected by a 4f imaging system to visualize inside the sample. In this manner, the diffuser functions as a phase scrambler (similar to the phase scrambler 132 or an of the phase scramblers shown herein) to produce a local phase change in a light beam. Extension and addition of dispersion compensation elements to the reference arm can be employed to account for the addition of lenses and the extension of the sample arm.

In another example, a SFOCT system can be built based on the iFusion system manufactured by Optovue Inc. for retinal imaging and approved by the Food and Drug Administration (FDA). With this ophthalmic OCT, the image plane need not be replicated because an image plane is accessible inside the original OCT probe. The ophthalmic implementation of SFOCT can be simpler than that for other tissue imaging, because less change in the sample arm is made and the reference may not need any change at all (e.g., there is no need to include a dispersion compensation element).

For both examples using a commercial system, local and time-varying phase shifts can be implemented by placing a moving ground glass diffuser (a phase scrambler) at the OCT image plane. The diffuser can be placed, for example, inside a mount and moved by a motor in a plane substantially perpendicular to the optical axis (i.e., propagation direction of the light beams), as described above. Images can be acquired several times while the light beam is imaging the same location on the sample but propagating through different locations on the diffuser. In this manner, the time-varying pattern of the diffuser changes the speckle pattern of the image. After averaging several frames, speckle noise can be reduced significantly.

In some embodiments, SFOCT images in the above described systems, except for the human retina images, can be acquired using a Ganymede High-Resolution SD-OCT system (ThorLabs, Newton, N.J.) in accordance with any of the methods described herein. In some such embodiments, the light source can be a super-luminescent diode (SLED or SLD) operating at 30 kHz with a center wavelength at 900 nm and a full bandwidth of 200 nm (Δλ=800-1000 nm), which can provide 2.1 μm axial resolution in water. Further, the spectrometer can acquire 2048 samples for each A-scan. In some embodiments, at the beginning of each acquisition, the OCT can be programmed to measure the spectrum of the SLD for 25 times. These measurements can be used for the reconstruction of the OCT signal. The first lens of the imaging system can provide a lateral resolution of about 8 μm (FWHM) and depth of field (DOF) of about 143 μm in water (LSM03-BB, ThorLabs, Newton, N.J.). The 4f configuration can be implemented using lenses that provide a lateral resolution of about 4.2 μm (FWHM) and DOF of about 32 μm in water (LSM02-BB, ThorLabs, Newton, N.J.).

Due to the modification of the sample arm, including increasing the arm length, inclusion of lenses for the 4f configuration, and inclusion of the diffuser, dispersion is introduced. Accordingly, the reference arm can be extended by approximately 10 cm and dispersion compensation elements can be added (2×LSM02DC). The reference arm extension can be accomplished by, for example, placing metal extension rods between the OCT probe and the reference mirror.

In some embodiments, a retrofit of a commercially-available system can employ a diffuser to produce a local phase change in the light beam. Thus, the diffuser is a phase scrambler, as described herein. In such embodiments, the phase scrambler can be a ground glass diffuser with anti-reflection (AR) coating on one side (e.g., Thorlabs, DG10-1500-B and DG10-2000-B). In other embodiments, a phase scrambler can be a 3 μm lapped diffuser, which can be created by further lapping a 1500 grit diffuser. The diffuser can be mounted by a custom motorized mount with XYZ translation (e.g., based on CXYZ1, ThorLabs, Newton, N.J.). A conventional manual mount can also be used (e.g., ST1XY-S, ThorLabs, Newton, N.J.). The phase scrambler can be moved by the motors and controlled through computer software (e.g., Thorlabs APT). The movement of the phase scrambler can be perpendicular to the direction of the scan. For example, if the light beam is scanned along the X direction on the sample, the phase scrambler is then moved along the Y direction, and vice the versa. The diffuser can be moved back and forth at a speed of, for example, 0.3 mm/s with a range of 6.5 mm. The acceleration of the movement can be, for example, 1.5 mm/s/s.

In some embodiments, the phase scrambler is placed within the Rayleigh range of the Gaussian beam of the OCT system. In practice, the phase scrambler can be adjusted along the propagation direction of the light beams until a satisfactory image is acquired.

In some embodiments, images produced in SFOCT are normally averaged over several images taken at different timing moments. This averaging should not limit the application of SFOCT, at least because image averaging is already widely used in conventional OCT systems to reduce photon and thermal noise. In addition, as the acquisition rates if detectors increase, obtaining multiple frames can be completed within a shorter and shorter time, therefore without imposing any additional limitation to the application of SFOCT.

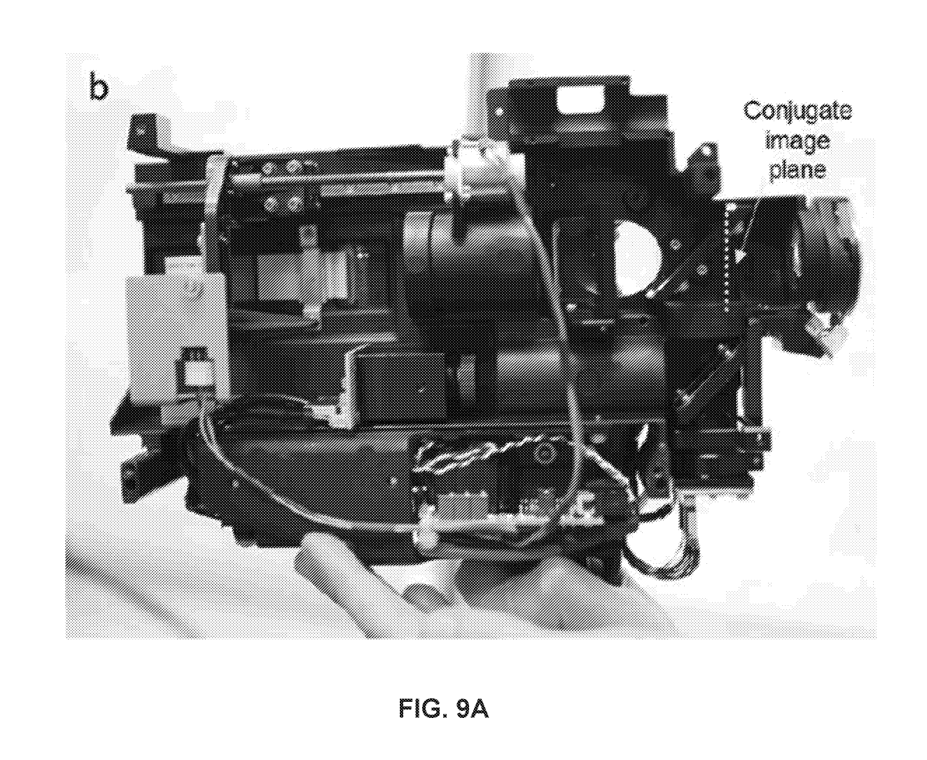

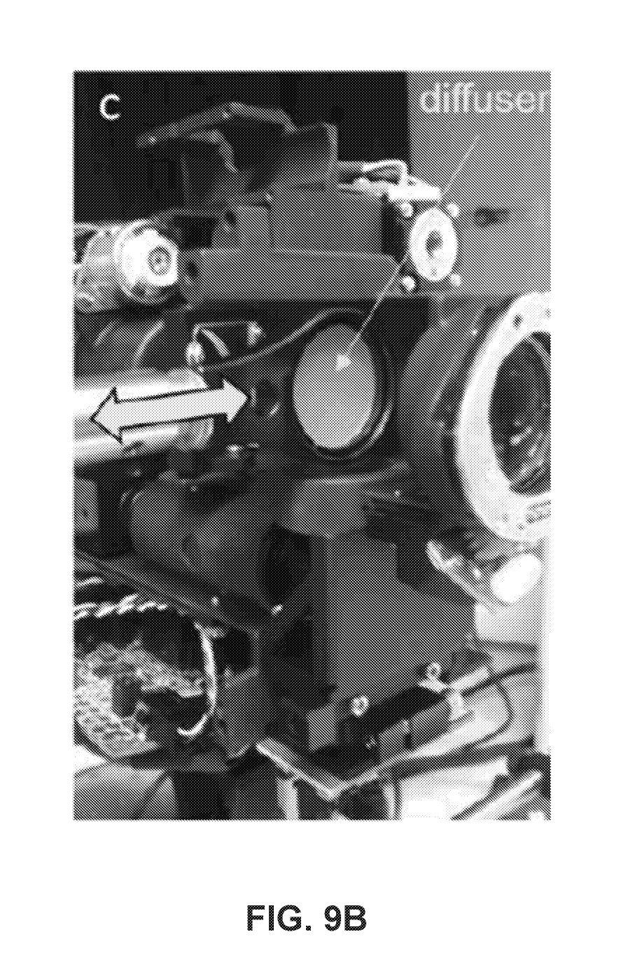

FIG. 9A is a photograph of the interior of the scan-head in the iFusion system described above. The conjugate image plane is marked by the dashed line. FIG. 9B is a photo of the scan-head with the diffuser placed in the conjugate image plane. The arrow shows the direction of the motion of the diffuser.

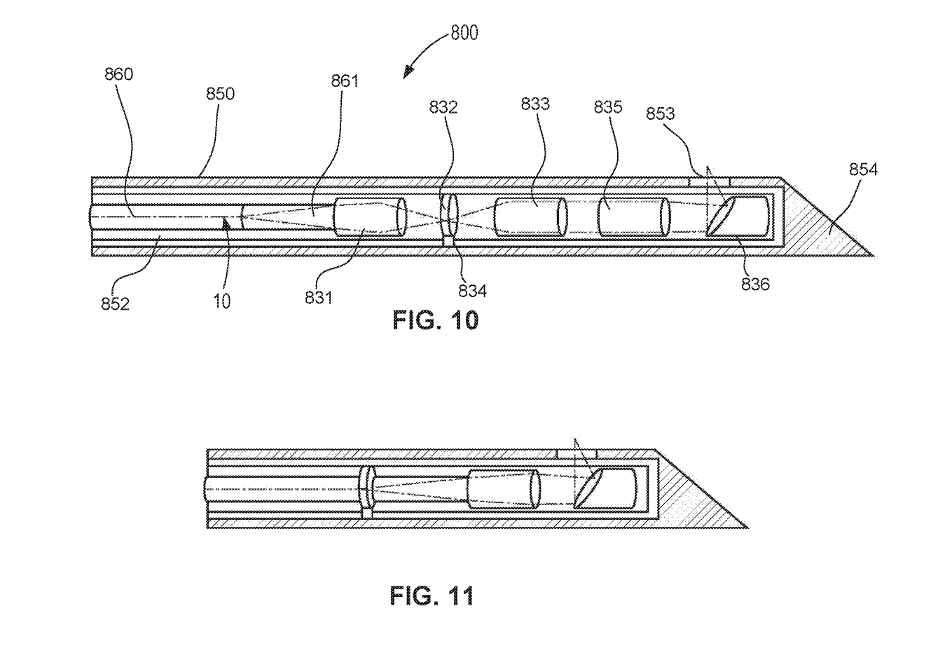

In some embodiments any of the optical systems, phase scrambler and methods described herein can be employed with an endoscopic imaging system. For example, in some embodiments, the free space optics (including the optical components, such as lenses, mirrors, transmission members or the like) described in any of the reference arms and/or sample arms described herein can be placed within an endoscope. For example, FIG. 10 shows a schematic illustration of an endoscopic SFOCT system 800 according to an embodiment. The endoscopic system 800 includes an elongated member 850, an optical transmission member 860, at least one lens 831, and a phase scrambler 832. The elongated member 850 includes a distal end portion 854, and is configured to be disposed within a bodily cavity (not shown). The distal end portion 854 can be configured and/or shaped to pierce or dilate bodily tissue. The elongated member 850 includes a side wall 851 that defines a lumen 852. The side wall 851 of the elongated member defines an opening 853 at the distal end portion 854.

The optical transmission member 860 can be any suitable optical component or structure to propagate light beams between a light source and/or a detector (not shown) and a sample (not shown) via the elongated member 850. Specifically, the optical transmission member 860 is disposed within the lumen 852, and conveys a sample light beam 10 therethrough. For example, in some embodiments, the optical transmission member 860 can be an optical fiber through which light can be propagated.

The sample light beam 10 can be produced by any suitable light source of the types shown and described herein, and is a spatially coherent light beam. The optical transmission member 860 also propagates a returned portion (not identified in FIG. 10) of the light scattered by the sample to a detector (not shown). The detector can be any suitable detector of the types described herein that detects and/or receives light scattered and/or reflected from the sample and any reference elements. Specifically, as described herein, the returned light portion from the sample and a returned light portion from a reference (not shown) are combined at the detector, which detects an interference pattern (when the system is properly aligned) produced by interference the returned light portions.

As shown, the system 800 includes a first lens 831, a second lens 833, and a third lens 835. The lenses, the optical transmission member 860, and the opening 853 of the elongated member 850 define at least a portion of a sample light path through which the sample light beam is conveyed to a sample (not shown). In some embodiments, the optical transmission member 860 can be an optical fiber coupled to the first lens 831 via a coupling member (or spacer) 861. The lenses can have any suitable configuration and/or arrangement within the elongated member 850. For example, in some embodiments the first lens 831 has a first focal length, the second lens 833 has a second focal length, and the third lens 833 also has a second focal length that matches that of the second lens. Thus, in such embodiments the three lens 831, 833, and 835 form a 4f configuration, as described above. In this manner, a conjugate image plane can be produced inside the elongated member 850 by using this lens arrangement. In other embodiments, however, the system 800 can employ any suitable lens configuration. Moreover, as shown, in some embodiments, an endoscopic system 800 can include a mirror 836 or other reflective element to propagate light through the opening 853 and to a sample.

The phase scrambler 832 is disposed at least partially in the sample light path, and reduces a spatial coherence of the spatially coherent light beam 10 that is propagated to the sample (e.g., via the opening 853). More particularly, as described herein, the phase scrambler 832 can produce a sample light beam having a spatially variable phase. In some embodiments, the phase scrambler 832 (and any of the phase scramblers and/or diffusers described herein) can include, but are not limited to, a ground glass element, a sandblasted glass element, an opal diffusing glass, or a holographic optical element, of the types shown and described herein.

In some embodiments, the phase scrambler 832 is disposed approximately at an image plane of a lens (e.g., the lens 831 or the lens 833) within the elongated member 850. Similarly stated, in some embodiments, the phase scrambler 832 is aligned with a focal plane associated with the sample and/or a lens (e.g., the lens 831 or the lens 833) within the elongated member 850. In some embodiments, the phase scrambler 832 is disposed within a Rayleigh range of the image plane associated with the sample and/or a lens (e.g., the lens 831 or the lens 833) within the elongated member 850. In this manner, the phase scrambler 832 acts to change the local phase of the sample light beam, as described above.

In some such embodiments, the system 800 includes an actuator 834 (or controller) to move the phase scrambler 832 within the image plane. In some such embodiments, the actuator 834 includes an actuator (not shown) to move the phase scrambler along a direction non-parallel (e.g., substantially perpendicular to) a propagation direction of the second portion of the spatially coherent light beam 10. In some embodiments, the actuator 834 is configured to rotate the phase scrambler 832 within the image plane. The actuator can be, for example, a motor that rotates the phase scrambler 832 continuously during a sampling operation. In other embodiments, however, the phase scrambler 832 can be at a fixed position within the elongated member 850, as described herein.

Although shown as including three lenses, in other embodiments, an endoscopic SFOCT system can include any suitable lens configuration. For example, FIG. 11 shows a schematic illustration of an endoscopic SFOCT system 900 according to an embodiment. The endoscopic system 900 includes an elongated member 950, an optical transmission member 960, a lens 931, and a phase scrambler 932. The elongated member 950 includes a distal end portion 954, and is configured to be disposed within a bodily cavity (not shown). The distal end portion 954 can be configured and/or shaped to pierce or dilate bodily tissue. The elongated member 950 includes a side wall 951 that defines a lumen 952. The side wall 951 of the elongated member defines an opening 953 at the distal end portion 954.

The optical transmission member 960 can be any suitable optical component or structure to propagate light beams between a light source and/or a detector (not shown) and a sample (not shown) via the elongated member 950. Specifically, the optical transmission member 960 is disposed within the lumen 952, and conveys a sample light beam 10 therethrough. For example, in some embodiments, the optical transmission member 960 can be an optical fiber through which light can be propagated.

As shown, the system 900 includes a single lens 931. The lens 931, the optical transmission member 960, and the opening 953 of the elongated member 950 define at least a portion of a sample light path through which the sample light beam is conveyed to a sample (not shown). Moreover, as shown, in some embodiments, an endoscopic system 900 can include a mirror 936 or other reflective element to propagate light through the opening 953 and to a sample.

The phase scrambler 932 is disposed at least partially in the sample light path, and reduces a spatial coherence of the spatially coherent light beam 10 that is propagated to the sample (e.g., via the opening 953). More particularly, as described herein, the phase scrambler 932 can produce a sample light beam having a spatially variable phase. In some embodiments, the phase scrambler 932 (and any of the phase scramblers and/or diffusers described herein) can include, but are not limited to, a ground glass element, a sandblasted glass element, an opal diffusing glass, or a holographic optical element, of the types shown and described herein. As shown, the phase scrambler 932 is disposed at the tip (or end surface) of the optical transmission member 960. Specifically, the phase scrambler 932 is disposed between a coupling member (or spacer) 961 and the optical transmission member 960.

As described above, in some embodiments any of the systems and methods described herein can include a movable phase scrambler that introduces a time-varying shift can into illuminating light beams in imaging systems. In some embodiments, any of the phase scrambler can be a transmissive optical member, such as a diffuser. To improve speckle reduction, the random phases introduced by the phase scrambler (or diffuser) can be evenly distributed between 0 to 2π at the beam waist. Stated differently, the surface height variation of the diffuser can be λ/Δn, where λ is the operating wavelength of the imaging system and Δn is the difference of refractive index between the diffuser and air. For example, to obtain this phase shift using a diffuser made of glass with a refractive index of 1.5 (NBK-7) and light sources with a center wavelength of 900 nm, the total thickness variation of the diffuser can span at least 1.8 μm. On the other hand, deflection of light by the diffuser, which is more probable in a ground glass diffuser with a wide thickness range, may reduce the OCT signal.

Thus, the amount of surface roughness of the transmissive phase scrambler (or diffuser) should be sufficient to introduce the desired local phase shift, while also minimizing power loss as the beam is propagated through the diffuser. Moreover, if the surface roughness (i.e., the peak-to-peak variation in the surface structures) exceeds more than about 5 microns, the resulting images become blurry. Said another way, if the variation in the surface finish is too great, the light beam will lose temporal coherence, and thus the axial resolution will be limited.

To evaluate different diffusers for use as phase scramblers, three types of diffusers are used and characterized with respect to their thickness and roughness using a 3D optical profiler and an atomic force microscopy (AFM). The first diffuser (also the roughest diffuser) is an off-the-shelf 1500 grit diffuser with AR coating (Thorlabs Inc.). The second diffuser is a custom made 2000 grit diffuser (Thorlabs Inc.). The third diffuser is made by further grinding (lapping) the 1500 diffuser with 3 μm particles.

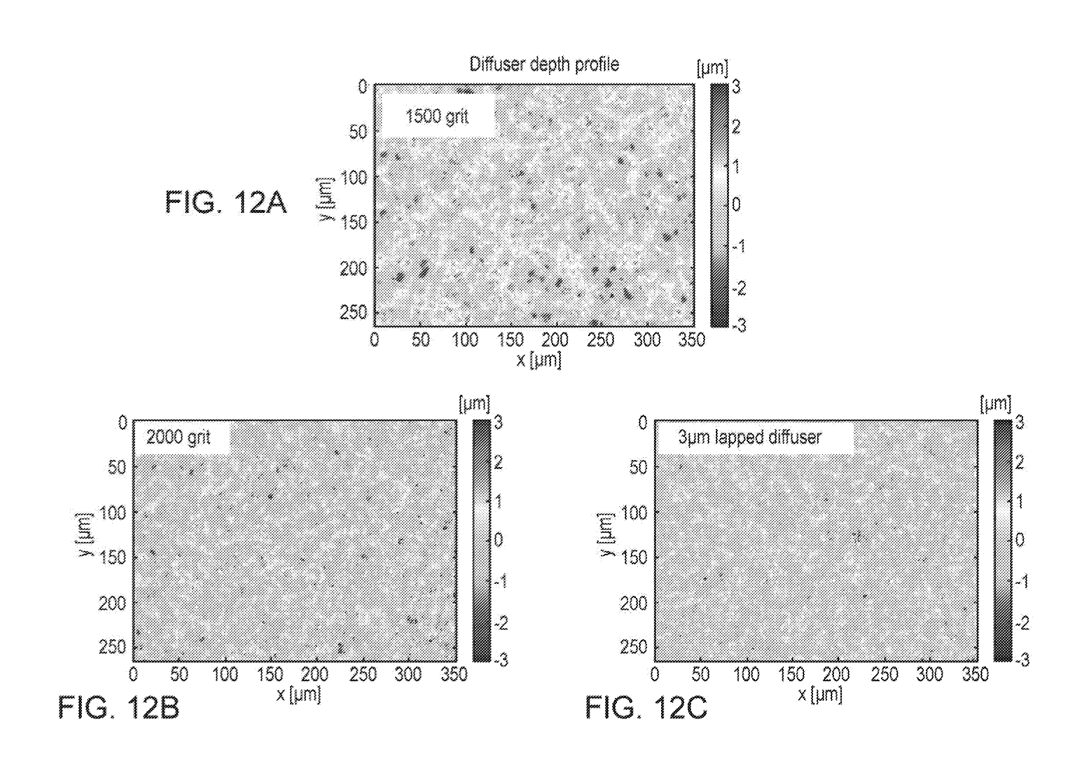

FIGS. 12A-12C show depth profiles of the 1500 grit diffuser, 2000 grit diffuser, and the lapped diffuser, respectively. The depth profiles of the diffusers can be measured with a 3D optical profilometer (e.g., neox, Sensofar) using a 50× magnification objective lens. The SensoSCAN program can be used for restore regions from which light may not be collected. The depth profiles in FIGS. 12A-12C show the surface roughness of the diffusers evaluated for suitability as phase scramblers in accordance with the systems and methods described herein: the 1500 grit diffuser has the roughest surface, followed by the 2000 grit diffuser. The lapped diffuser has the smoothest surface among the three.

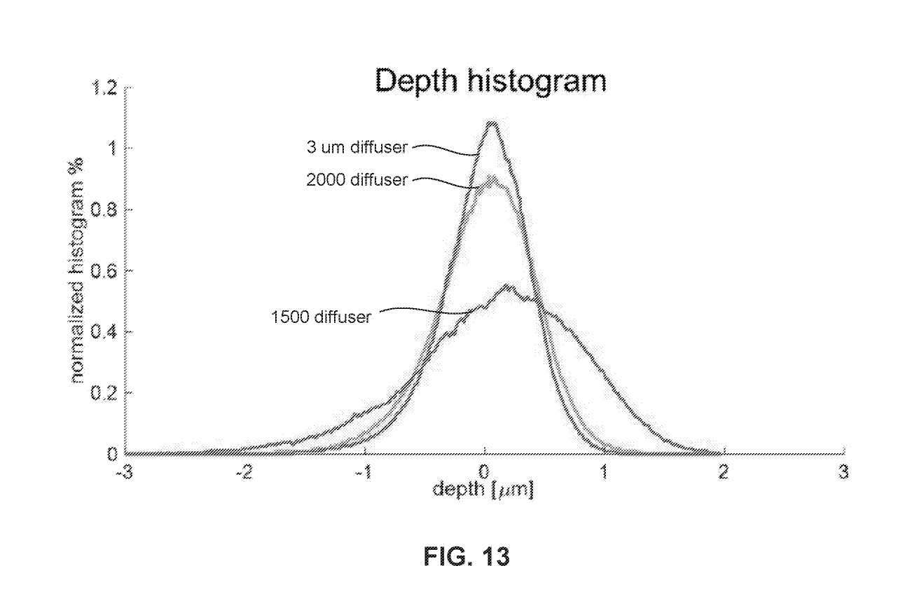

FIG. 13 shows depth histograms of the 1500 grit diffuser, 2000 grit diffuser, and the lapped diffuser, respectively. The depth histograms can be calculated by, for example, the SensoSCAN program. Depth histograms can be a more quantitative way to show the surface roughness. In general, narrower width of the histogram means that more surface points have a depth that is close to the central depth (or most probably depth). Accordingly, narrower width normally means a smoother surface. From FIG. 13, it can be seen that the 1500 grit diffuser has the roughest surface, the 2000 grit has the second roughest, and the lapped diffuser is the smoothest.

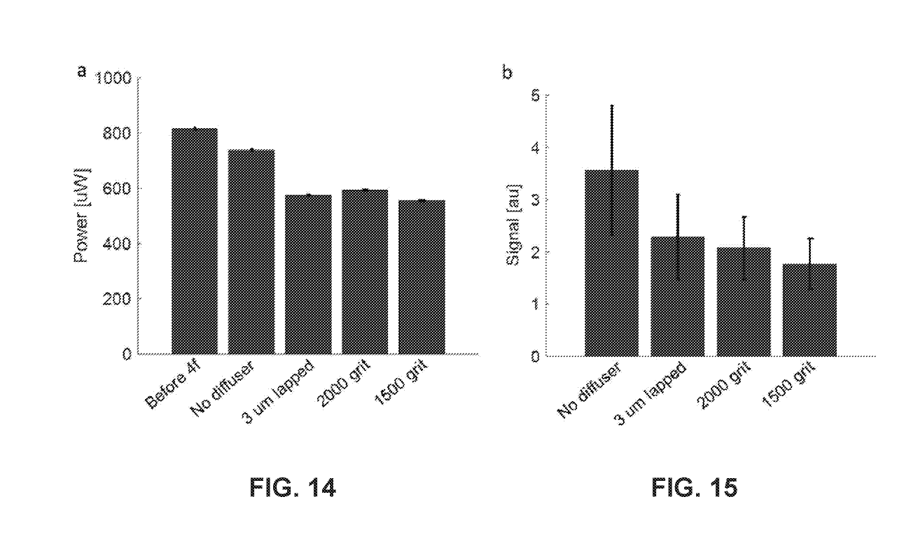

As discussed above, implementing SFOCT with diffusers having a different surface profile can have different effects on the optical power on the sample, the signal (also referred to as the OCT signal), and the lateral resolution. For example, FIG. 14 shows the optical power levels on the sample when different diffusers are used in an illustrating example and Table 1 below summarizes the data. The optical power measured for three diffusers is also compared to conventional OCT, which does not include a diffuser (or any phase scrambling, as described herein). The optical power can be measured by placing a power meter at the focal plane of the scan lens while scanning at a single point at the center of the field of view. At least 100 consecutive measurements can be acquired for a time period of 30-60 seconds. The measurement can be performed at the center wavelength of the source at 900 nm. The OCT measurement refers to the original probe, without any addition. The measurement named “no diffuser” refers to the SFOCT system, with the addition of two lenses, but without a diffuser. In the non-retinal system, the power on the sample may be reduced by 9% due to the 4f imaging system and an additional reduction of 22%, 20% and 25% due to the 3 μm lapped, 2000 grit and 1500 grit diffusers, respectively.

TABLE 1
Optical power on the sample when different diffusers are used

| | | | Relative | | Relative | |
|---|---|---|---|---|---|---|
| | Mean | STD | trans- | Power | trans- | Diffuser |
| | [uW] | [uW] | mission | loss | mission | power loss |
| OCT | 814.75 | 4.24 | | | | |
| No diffuser | 738.25 | 2.61 | 91% | 9% | | |
| 3 um lapped | 574.85 | 1.83 | 71% | 29% | 78% | 22% |
| 2000 grit | 593.55 | 1.9 | 73% | 27% | 80% | 20% |
| 1500 grit | 555.7 | 2.05 | 68% | 32% | 75% | 25% |

FIG. 15 shows signal levels when the different diffusers are used in an illustrating example, and Table 2 summarizes the data. The signal intensity can be measured on images of a PDMS+TiO₂ phantom structure with 100 B-scan averages. The regions selected for the measurements were at the same depth in the phantom structure, the same location relative to the focal plane, and the same position on the screen. This procedure eliminated the effect of absorption, focusing and signal roll-off. The measured values are on a linear scale and in arbitrary units. The relatively high standard deviation in the signal intensity may be attributable to the absorbance inside of the region selected for this measurement. The decrease in signal intensity due to the diffusers can be greater than the decrease in power on the sample because the signal is created by light that is travelling twice trough the diffuser.

FIG. 15 and Table 2 show that the OCT and SFOCT signals have an average signal loss of 36%, 42% and 50% with the three diffusers (phase scramblers) compared to OCT. Even though in the example shown in FIG. 15 and Table 2, SFOCT results in a lower signal to noise ratio, the system and methods of employing phase scrambling using a diffuser are still able to produce images with a greater sensitivity than that in conventional OCT. In addition, the reduction in signal intensity can be compensated for by increasing the power on the sample by increasing the power of the laser and diverting a percentage of the light from the reference arm to the sample arm.

TABLE 2
Signal levels when different diffusers are used

| | mean signal | Standard | |
|---|---|---|---|
| | intensity [au] | deviation [au] | Signal loss |
| no diffuser | 3.559 | 1.238 | |
| 3 um lapped | 2.285 | 0.812 | 36% |
| 2000 grit | 2.075 | 0.598 | 42% |
| 1500 grit | 1.769 | 0.486 | 50% |

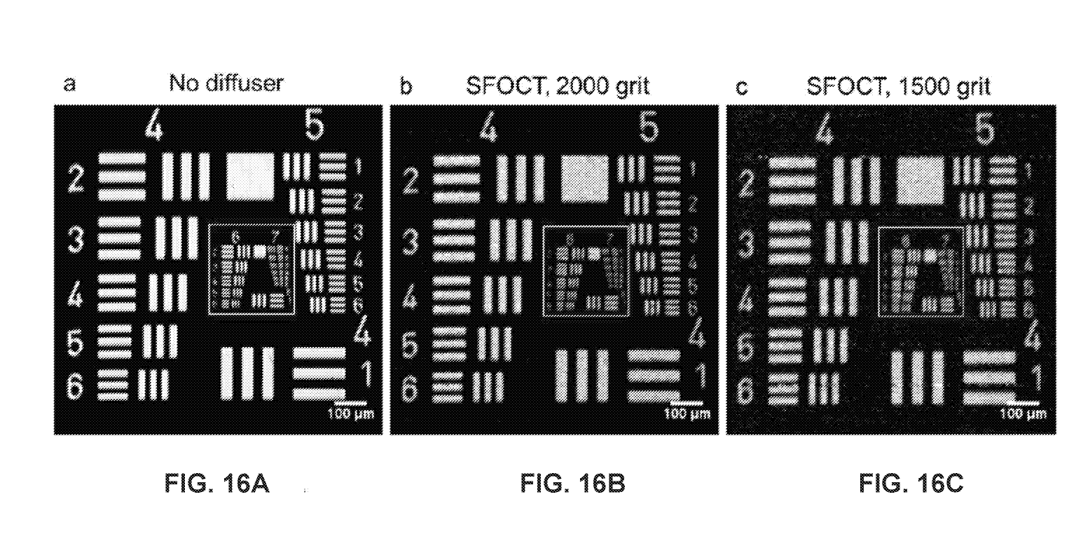

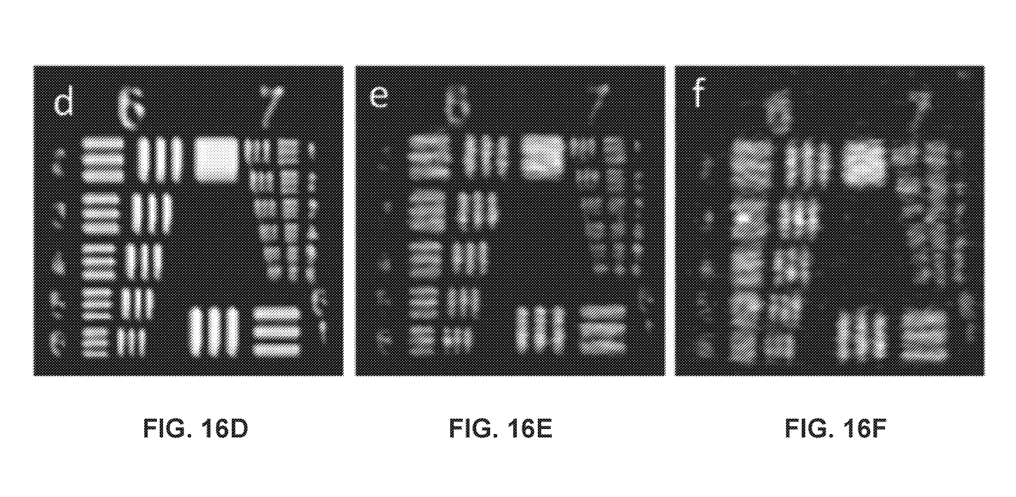

FIGS. 16A-16F show resolution measurements of SFOCT with the three different diffuser types in an illustrative example, and Table 3 summarizes the data. The lateral resolution of SFOCT can be measured using a resolution test target (e.g., 1951 USAF Glass Slide Resolution Target, Edmund Inc.). FIGS. 16A-16C show images of the USAF target acquired by SFOCT without a diffuser (effectively a regular OCT), SFOCT with a 2000 grit diffuser, and SFOCT with a 1500 grit diffuser, respectively. FIGS. 16D-16F show closed-up views of the rectangular regions marked in FIGS. 16A-16C, respectively. The results show an increase of 35.3% and 6.2% in the size of the point spread function, with the 1500 grit and 2000 grit diffuser. The difference between the two diffusers can be a result of signal intensity variations, caused by the increased roughness of the 1500 grit diffuser, which may not be completely removed by the 54 averages used to create these images.

TABLE 3
Resolution measurement of SFOCT using different diffusers

| | Ver- | Line | | Hori- | Line | | |
|---|---|---|---|---|---|---|---|
| | tical | pairs | Pixel | zontal | pairs | Pixel | |
| | sepa- | per | FWHM | sepa- | per | FWHM | PSF |
| | ration | mm | [um] | ration | mm | [um] | increase |
| No diffuser | 6 (6) | 114 | 8.8 | 7 (3) | 161 | 6.2 | |
| SFOCT 2000 | 6 (6) | 114 | 8.8 | 7 (2) | 144 | 6.9 | 6.18% |
| SFOCT 1500 | 6 (1) | 64 | 15.6 | 6 (6) | 114 | 8.8 | 35.27% |

Although the roughest diffuser (1500 grit) may reduce the OCT signal and the lateral resolution the most (35.3% increase in the size of the point spread function, versus 6.2% in the 2000 grit diffuser), it can achieve the best overall performance in terms of speckle removal and appearance of small detail in tissue. This tradeoff may be avoided by careful design and fabrication of a designated diffuser.

Although the phase scramblers described herein can include a diffuser of the types shown and described herein, and can be moved relative to a light path to introduce a local phase difference that is changed between image samples, in other embodiments, any suitable phase scrambler can be used in any of the systems and methods described herein. For example, in some embodiments, any of the systems and methods described herein can include a phase scrambler that is at a fixed location. In other embodiments, any of the systems and methods described herein can include a phase scrambler does not transmit light therethrough. For example, in some embodiments, a phase scrambler, such as the phase scrambler 132, includes a spatial light modulator configured to change the diffusion of a portion (i.e., a split beam) of spatially coherent light via at least one of a mechanical force, an electrical field, a magnetic field, or a thermal field.

Various types of spatial light modulators can be used as a phase scrambler. In one example, the spatial light modulator can be an electrically addressed spatial light modulator (EASLM). The diffusion in an electrically addressed spatial light modulator can be created and changed electronically (similar to most electronic displays). EASLMs usually receive input via a conventional interface such as Digital Visual Interface (DVI) or Video Graphics Array (VGA) input. An example of an EASLM is the Digital Micromirror Device using ferroelectric liquid crystals (FLCoS) or nematic liquid crystals (Electrically Controlled Birefringence effect). In another example, the spatial light modulator can be an optically addressed spatial light modulator (OASLM). The diffusion in an optically addressed spatial light modulator, also known as a light valve, can be created and changed by shining light encoded with an image on the front or back surface of the OASLM. A photosensor can be employed to allow the OASLM to sense the brightness of each pixel and replicate the pattern in the encoded light using liquid crystals. Typically, as long as the OASLM is powered, the diffusion pattern is retained even after the light is extinguished. An electrical signal can be used to clear the whole OASLM at once.

Effective Resolution of SFOCT

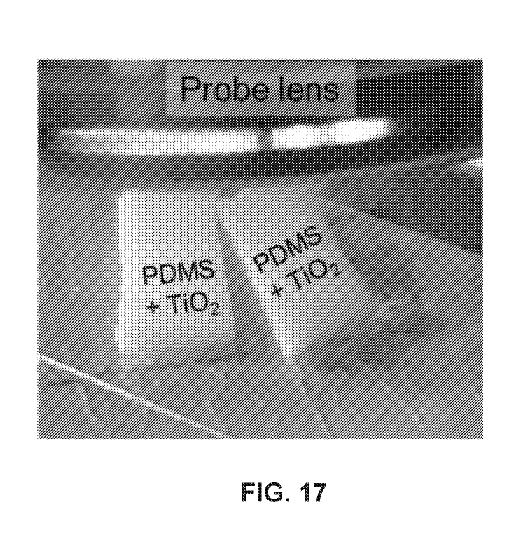

One advantage of SFOCT using the systems and methods described herein can be the improved effective resolution so that closely-spaced scatters or other features can be distinguished (resolved). This improved effective resolution can be demonstrated and quantified by imaging an infinitesimally small gap. For example, FIG. 17 is a photo of a structure forming a sample gap that can be used to characterize the effective resolution of a SFOCT. The gap includes two plates made of titanium dioxide (TiO₂) powder dispersed in Polydimethylsiloxane (PDMS). The infinitesimally small gap can then be constructed by bringing together two rectangular pieces of the phantom at an angle.

More specifically, four 5 mL agarose phantoms embedded with various scattering agents can be created using a stock solution of 1% agarose in water. The agarose solution can be prepared on a hot plate with a magnetic stirrer and kept at a constant temperature of 60° C. to prevent curing or clumping. Three different scattering agents were employed: 0.3-1 μm TiO₂ rutile powder (e.g., Atlantic Equipment Engineers, Upper Saddle River, N.J.), 21 nm (primary particle size) TiO₂ anatase nanopowder (e.g., Sigma Aldrich Co. St. Louis, Mo.), and OD 500 gold nano-rods (GNRs) with peak absorption at 745 nm. Two phantoms were made with a low and a high concentration of GNR, respectively. The high concentration phantom included 100 μL of GNR for every 5 mL of base, and the low concentration used 50 μL of GNR for every 5 mL of base. For phantoms with TiO₂ as the scattering agent, 0.009 grams of the TiO₂ were ultrasonically dispersed in 1 mL Millipore water using a water bath sonicator to prevent aggregation. The solution can be sonicated for four 30 second intervals with a two-minute gap between each round to prevent overheating. Scattering agents can be slowly added to 5 mL of uncured agarose at 60° C. with continuous stirring. The final solution can be stirred for one minute before being poured into 5 mL plastic petri dishes. Two hours or longer curing were carried out before being used in SFOCT for imaging.

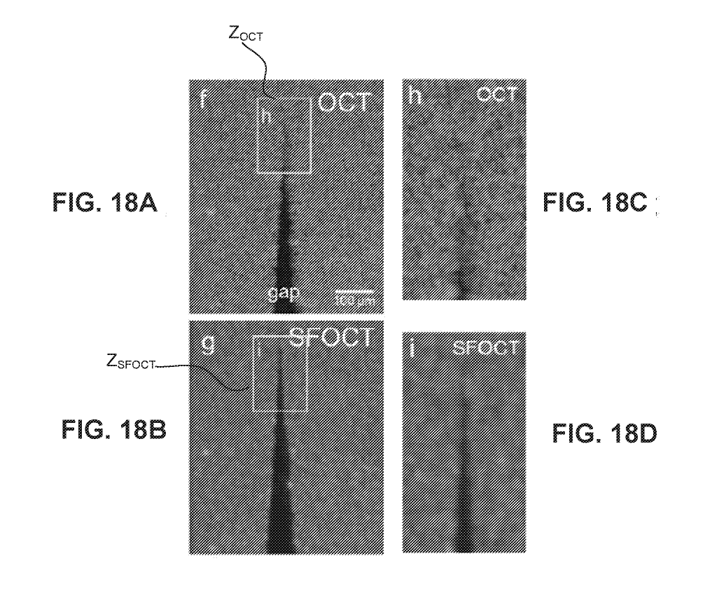

FIGS. 18A-18D show both OCT and SFOCT images of an example gap prepared according to the methods described above. More specifically, FIGS. 18A and 18B show OCT and SFOCT en face scans inside the phantom, respectively. FIGS. 18C and 18D show close-up views of the rectangular regions marked as Z_{OCT} and Z_{SFOCT}, respectively, in FIGS. 18A and 18B. The gap that is clearly visible in SFOCT (FIG. 18D) is not clearly visible in the OCT image due to speckle noise.

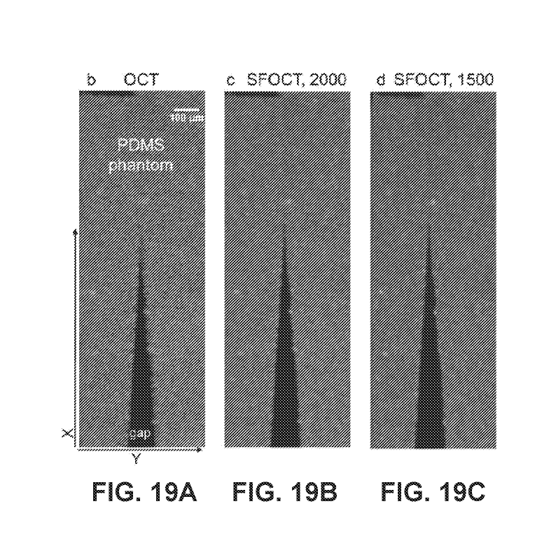

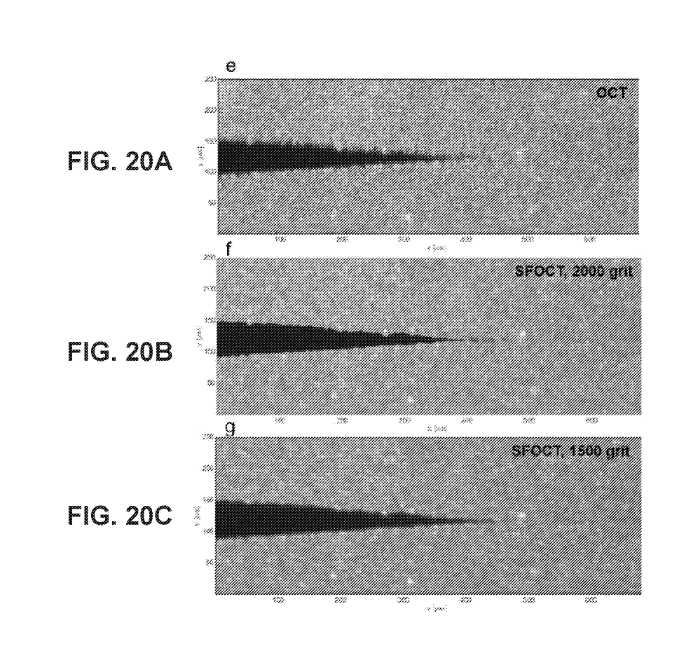

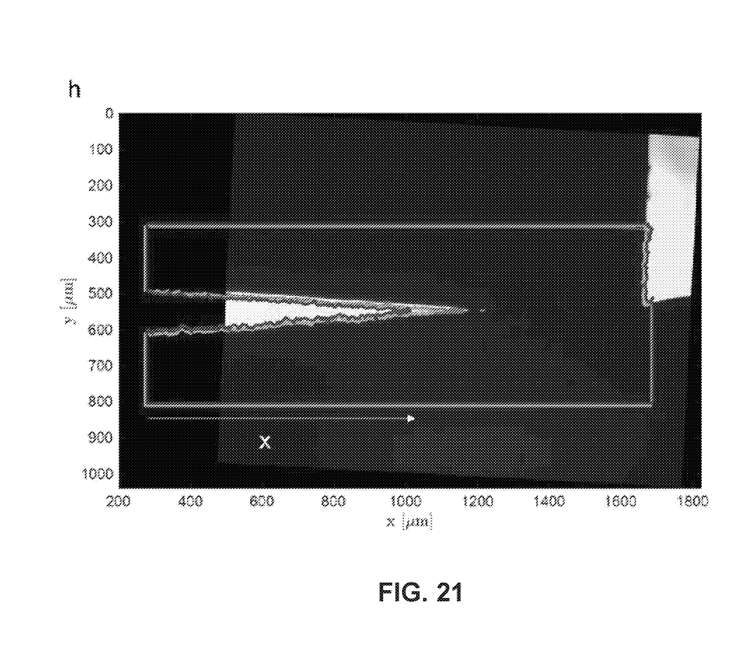

FIGS. 19A-19C show images of the gap using OCT, SFOCT with 2000 grit diffuser, and SFOCT with 1500 grit diffuser, respectively. FIGS. 20A-20C show OCT and SFOCT scans along with the lines which represents the segmentation boundary between the phantom and the gap. It can be seen from these images that SFOCT can resolve finer structures compared to OCT, and SFOCT using 1500 grit diffuser has a higher resolution that achieved in SFOCT using a 2000 grit diffuser. FIG. 21 shows a compilation of registration of the segmentation boundaries with an image of the phantom taken with a bright-field microscope (10×). Good agreement can be seen between the microscope image and the SFOCT measurements.

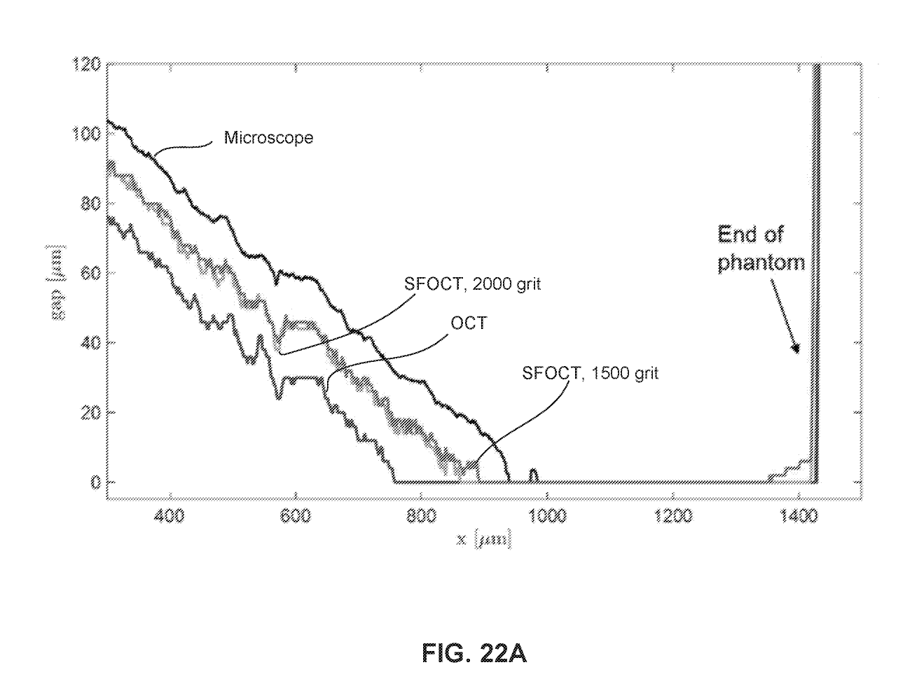

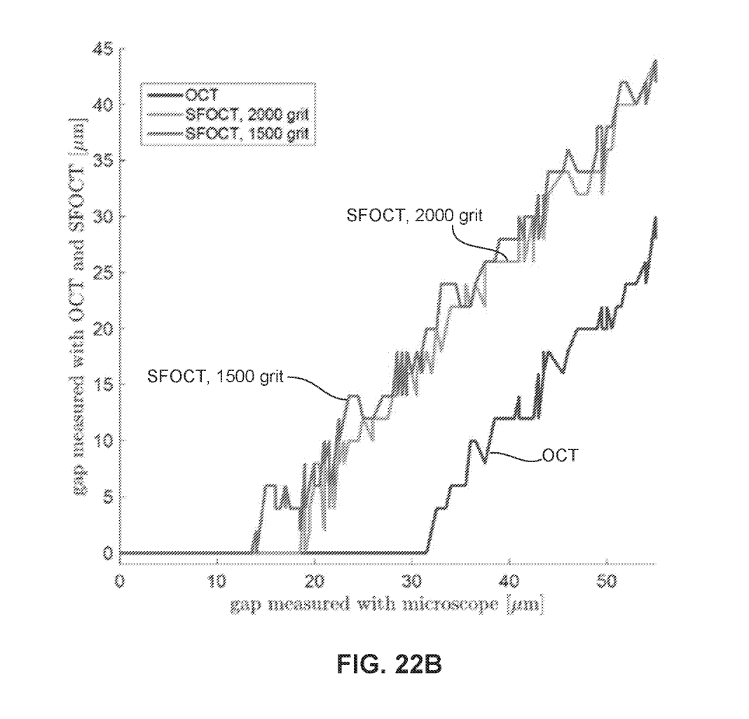

FIG. 22A shows the size of the gap as a function of location (x) as measured by different methods, including OCT, SFOCT using a 2000 grit diffuser, SFOCT using a 1500 grit diffuser, and microscope. FIG. 22B shows the size of the gap in the OCT and SFOCT images (as shown in FIG. 22A) plotted as a function of the size of the gap measured in the microscope image. The difference in visibility between OCT and SFOCT is clearly shown. The effective resolution in SFOCT is 2.5-fold better than OCT.

Although the lateral resolution of SFOCT can be lower compared to that in OCT when measured on a glass test chart (e.g., shown in FIGS. 16A-16F), the effective resolution of SFOCT can be significantly superior in turbid media, which is conventionally dominated by speckle. A 250% increase in the effective resolution is measured by SFOCT compared to conventional OCT, and it can be reasonable to infer that this increase would be even higher in lower contrast images.

Pixel Value Statistics in SFOCT

OCT speckle noise normally follows a Rayleigh distribution. This can be problematic since the Rayleigh statistics may dominate the image and obscure real features within the sample. SFOCT according to the embodiments described herein can reduce speckle noise by shifting the pixel value statistics from a Rayleigh distribution toward the expected distribution of scatters in a sample.

Speckle contrast can be theoretically and experimentally reduced by (M)^{1/2} using SFOCT conducted by the systems and according to the methods described herein. Since each image can be acquired at a similar angle, sample position, and illumination wavelength, increasing the number of compounded images normally does not correlate to an inherent degradation in resolution. Because increasing the number of uncorrelated images does not reduce resolution, it is possible to create many images and subsequently eliminate speckle noise entirely. An approximate mathematical description of this phenomenon is given by:

$$I = \frac{1}{M} {\sum }_{m = 1}^{M} \vert {\sum }_{n = 1}^{N} {a}_{n} {e}^{i {ϕ}_{n}} {e}^{i {θ}_{n m}} \vert \qquad ( 1 )$$

In which I is the pixel value after averaging M scans obtained at different times and with different local phase shifts. N is the number of scatters inside a voxel, with scattering amplitudes aₙ for the n^{th} scatterer and locations that cause a relative phase shift of φₙ. θₙₘ is the local phase shift in the location of scatterer n, and which changes in time. In conventional OCT θₙₘ is constantly equal to zero; in SFOCT, however, it is a random variable with a uniform distribution between 0 and 2π.

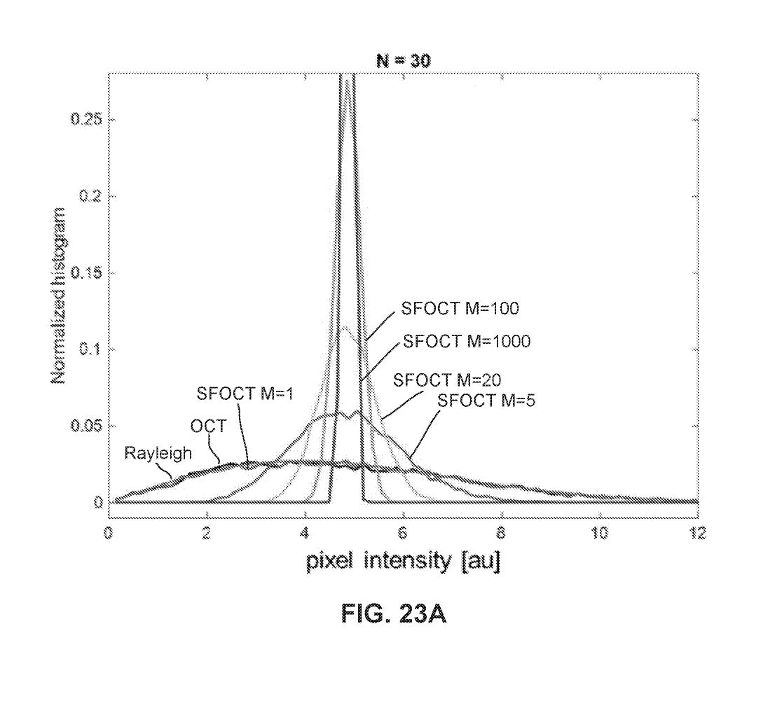

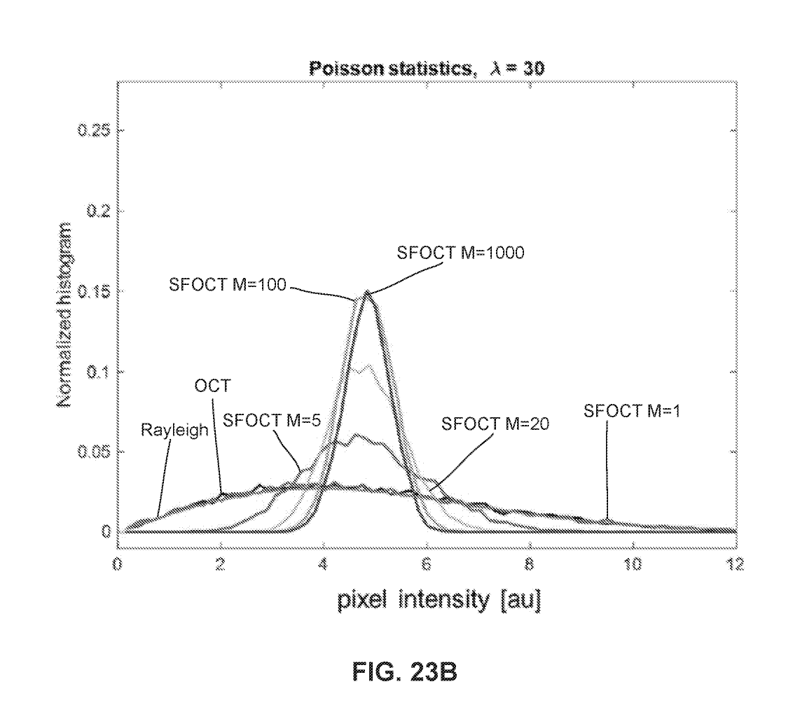

FIG. 23A shows normalized histograms of the pixel values obtained with a Monte-Carlo simulation of equation (1) with 30 particles in each voxel. FIG. 23B shows the number of particles in each voxel randomly chosen from a Poisson distribution with λ=30. The Monte-Carlo simulation considers 10,000 voxels. The number of particles in each voxel is either 30 or randomly chosen from a Poisson distribution with λ=30. The scattering amplitude is constant and equal to 1. The phase contributed by each particle, φₙ, is a random variable with a uniform distribution between 0 and 2π.

FIGS. 23A-23B show that increasing the number of averages narrows the pixel value distribution, thereby reducing the noise. In the case of a random number of particles in each voxel, the underline pixel intensity variation persists with increased averaging owing to the actual variation of scatterers within each voxel (or resolution volume). This may be the reason why the distribution does not continue to narrow when increasing the number of averages from 100 to 1000.

The shift of statistics can be experimentally demonstrated by measuring pixel value statistics of a phantom made of gold nanorods (GNRs) disposed in an agarose gel. Similar to the phantoms made with TiO₂, owing to the high backscattering of the metallic particles and their high concentration, these phantoms can be useful models for turbid media and produce speckle statistics as expected for conventional OCT imaging.

The pixel value statistics obtained with SFOCT resemble a Poisson distribution in which each event contributes a value that is equal to the backscattering of a single GNR. This distribution more closely matches the expected distribution of GNRs randomly dispersed within the phantom. As predicted by equation (1), increasing the number of averages reduces speckle noise and thus reduces the broadness of the distribution of pixel values. Because the phantoms are composed of a random spatial distribution of particles, a uniform signal is not expected when speckle noise is eliminated. Because SFOCT is capable of eliminating speckle, it can more closely approximate the actual distribution of scatterers in the sample.

As mentioned above, conventional OCT speckle noise follows a Rayleigh distribution. SFOCT reduces speckle and shifts the pixel value statistics from a Rayleigh distribution toward the expected distribution of scatterers in a sample, which is a Poisson distribution in which each event contributes a value that is equal to the backscattering of a single GNR. The two expressions for the two distributions are:

$$\begin{matrix}{p}_{rayleigh} ( I ) = \frac{π I}{2 {〈 I 〉}^{2}} exp [ - \frac{π {I}^{2}}{4 {〈 I 〉}^{2}} ] & ( 2 ) \\ {p}_{poisson} ( k ) = \frac{{λ}_{p}^{k} {e}^{- {λ}_{p}}}{k !} , {p}_{poisson} ( I ) = {I}_{p} \times {p}_{poisson} ( k ) & ( 3 )\end{matrix}$$

In which I is the pixel value and [symbol] I [symbol] the average pixel intensity. k is the number of particles in a voxel, which can be assumed to contribute equally to the OCT signal and λₚ is the average number of particles in a voxel. To obtain λₚ the histogram of the pixel values is fit to a Poisson distribution. From that, the contribution of each equivalent particle to the OCT signal is derived as Iₚ= [symbol] I [symbol] /λₚ.

To further validate that SFOCT conducted using the systems and in accordance with the methods described herein removes speckle, experimental data can be compared to the \sqrt{M} model. In the case of an inherent signal variation, the reduction in the normalized standard deviation (STD), can be described by:

$$\begin{matrix}C = \frac{σ}{〈 I 〉} & ( 4 a ) \\ {C}^{2} {〈 I 〉}^{2} = {σ}^{2} = {σ}_{0}^{2} + {σ}_{speckle}^{2} & ( 4 b ) \\ {σ}_{speckle}^{2} = \frac{{σ}_{speckle , 0}^{2}}{M} & ( 4 c )\end{matrix}$$

In which σ is the measured STD in a region of interest, [symbol] I [symbol] the average pixel intensity and C is the normalized STD which includes the speckle contrast and the inherent signal variation of the sample. σ² is the variance in pixel values, σ₀² is the intrinsic variation in signal caused only by the variation in number of particles in a voxel, and σ_{speckle}² is the variance of the speckle noise, which reduces by a factor of M during compounding.

Equations (4a)-(4c) show that the normalized speckle, as defined below, should reduce by \sqrt{M}:

$$Normalized speckle = \sqrt{\frac{{σ}^{2} - {σ}_{0}^{2}}{{σ}_{speckle , 0}^{2}}} = \frac{1}{\sqrt{M}} \qquad ( 5 )$$

On a logarithmic:

$$log ( \sqrt{\frac{{σ}^{2} - {σ}_{0}^{2}}{{σ}_{speckle , 0}^{2}}} ) = - \frac{1}{2} log ( M ) \qquad ( 6 )$$

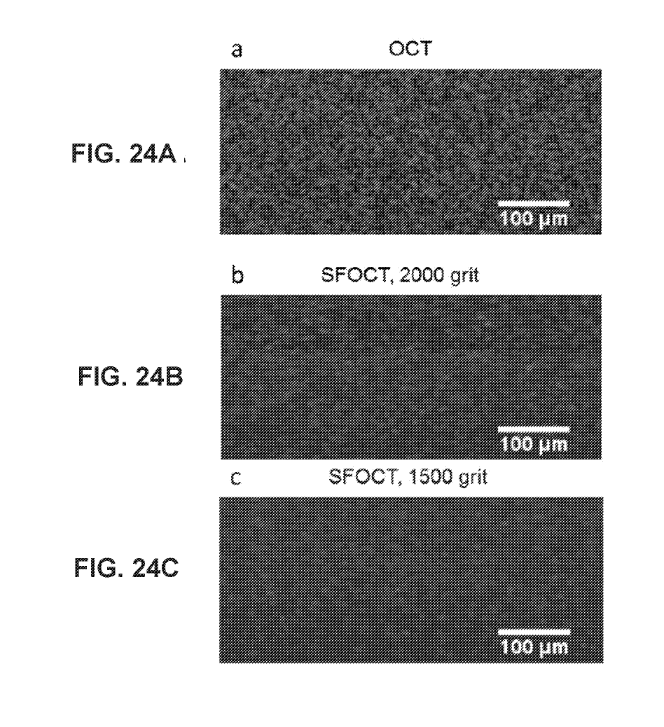

FIGS. 24A-24C show images of phantoms made of GNRs dispersed in agarose, acquired using OCT, SFOCT with 2000 grit diffuser, and SFOCT with 1500 grit diffuser, respectively. The OCT image shows a combination on speckle noise and the signal variation due to the random distribution of GNRs in the phantom. The SFOCT images show only the latter.

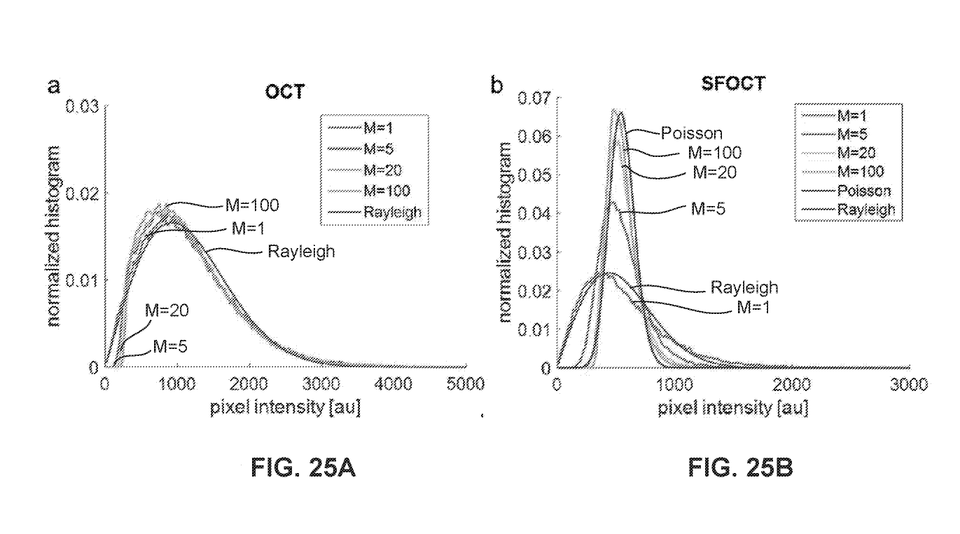

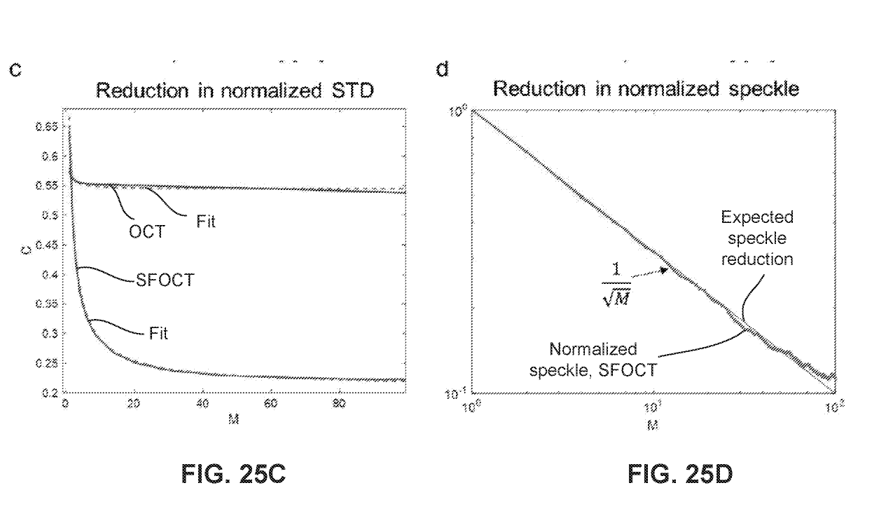

FIGS. 25A-25B show statistical analysis of pixel values of scans of a GNR phantom obtained with OCT and SFOCT, respectively, with a 1500 grit diffuser. The OCT image is dominated by speckle noise and the distribution of pixel values is approximately a Rayleigh distribution, which persists with averaging. In the SFOCT image as shown in FIG. 25B, increasing the number of averages quickly narrows the distribution of pixel values and moves it towards a Poisson distribution, which expresses the probability of a given number of GNRs to be present in a single voxel.

FIGS. 25C-25D show reduction in normalized STD versus the number of averages, M, for OCT and SFOCT, respectively. FIG. 25C shows that conventional OCT images exhibit negligible reduction in normalized standard deviation even with extensive averaging (M=100). In comparison, SFOCT imaging, according to the embodiments described herein, with equivalent averaging shows a significant reduction (approximately 60%) in the normalized standard deviation. Furthermore, the normalized speckle obtained using SFOCT exhibits a 90% reduction when M=100, which is consistent with the expected square root behavior (see, e.g., FIG. 25D, equation (6)). The values for σ₀² and σ_{speckle}² can be found using a non-linear least squares fit to equation (4b).

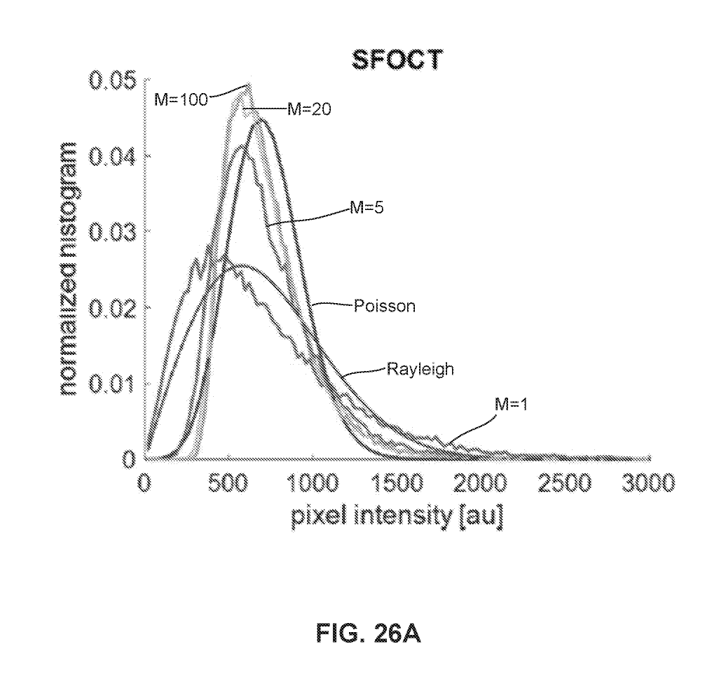

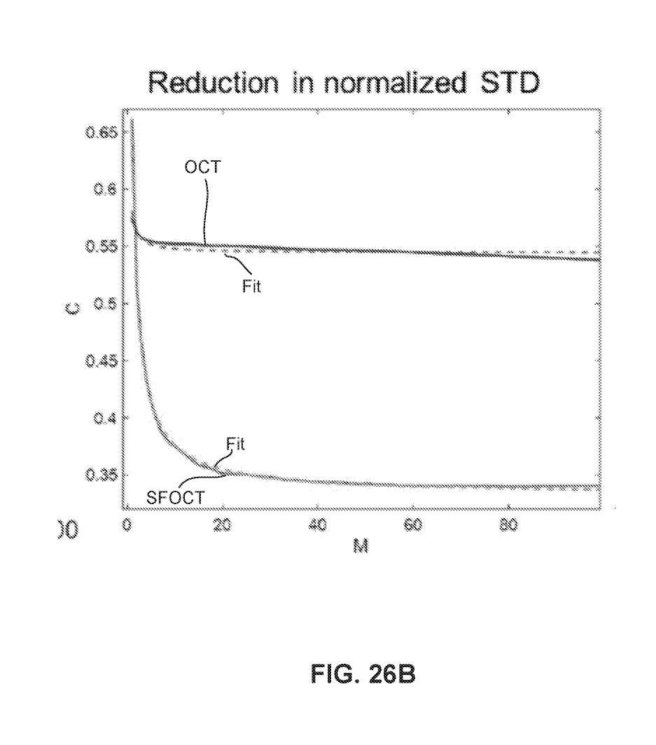

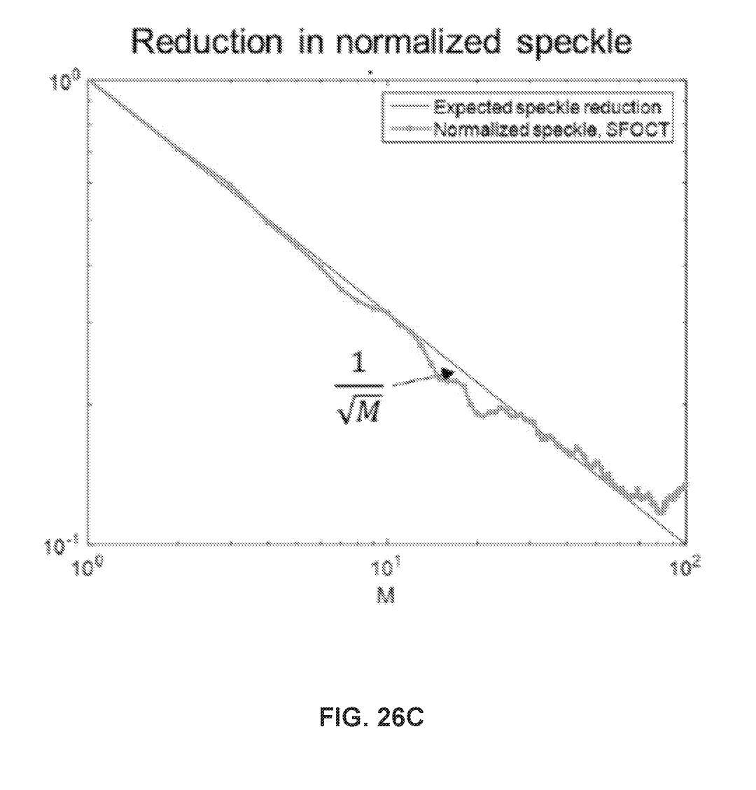

FIGS. 26A-26C show analysis of pixel values obtained with SFOCT with a 2000 grit diffuser. FIG. 26A shows normalized histogram of pixel values that demonstrate the transition from speckle statistics (Rayleigh) towards a Poisson distribution as the number of averages M increases. The Poisson distribution expresses the probability of a given number of GNRs to be present in a single voxel. FIG. 26B shows the reduction in normalized STD versus the number of averages, M, for OCT and SFOCT. The reduction in the normalized STD is significantly larger in SFOCT versus OCT, and follows the theoretical dependence on M as expressed in equation (6). The reduction in the normalized STD when imaging with the 1500 grit diffuser is greater compared to the 2000 grit diffuser. FIG. 26C shows the reduction of normalized speckle as defined by equation (5) follows 1/\sqrt{M}, as expected.

Imaging Fine Structures Using SFOCT

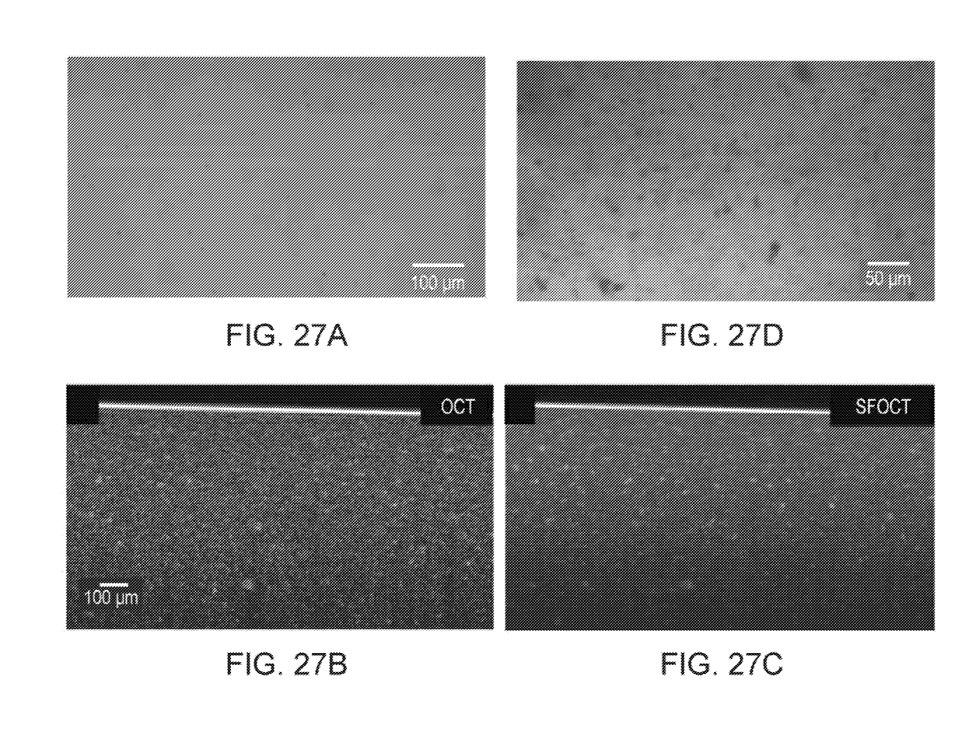

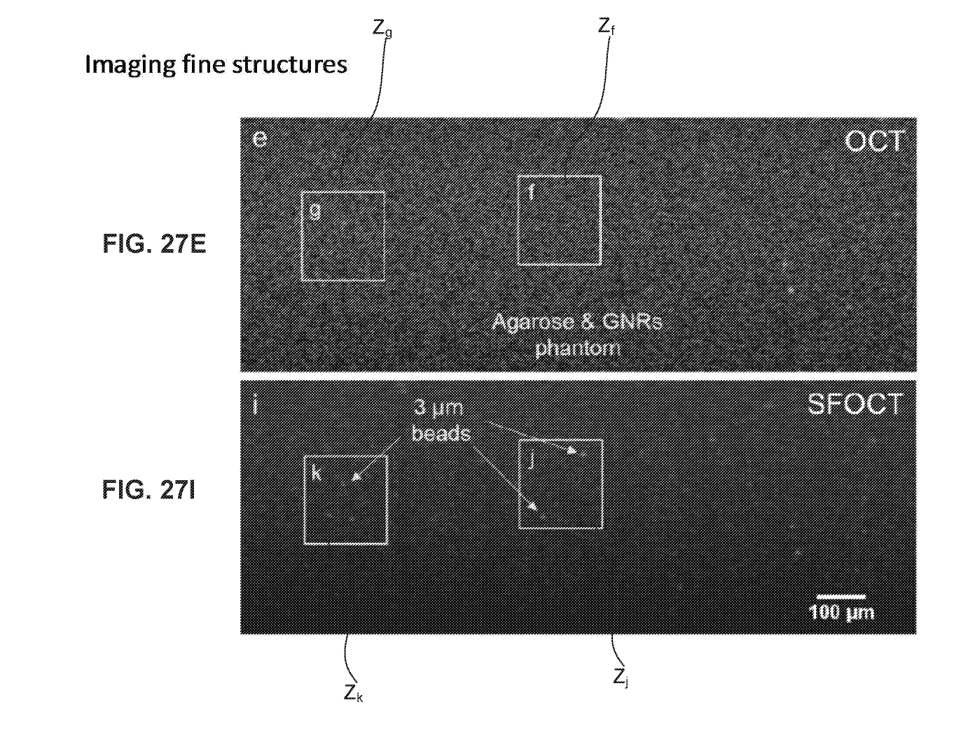

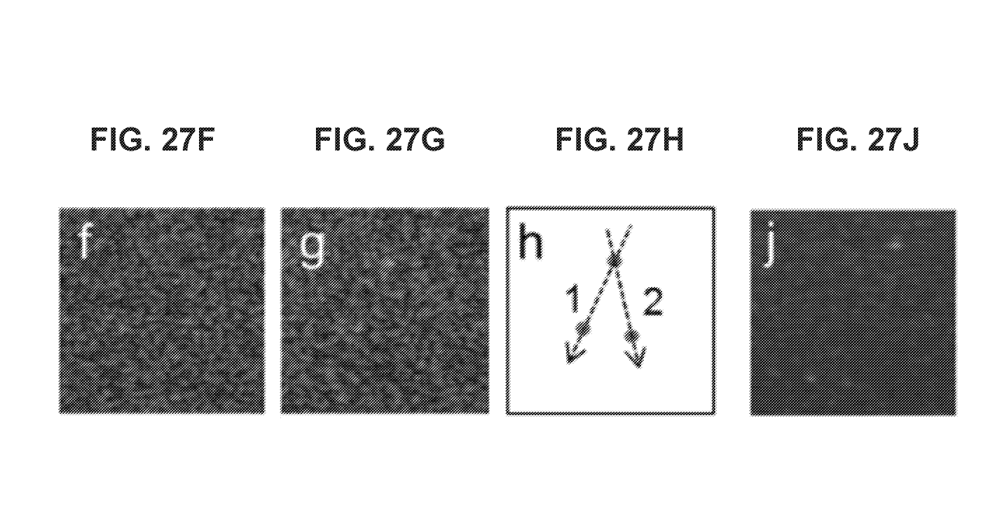

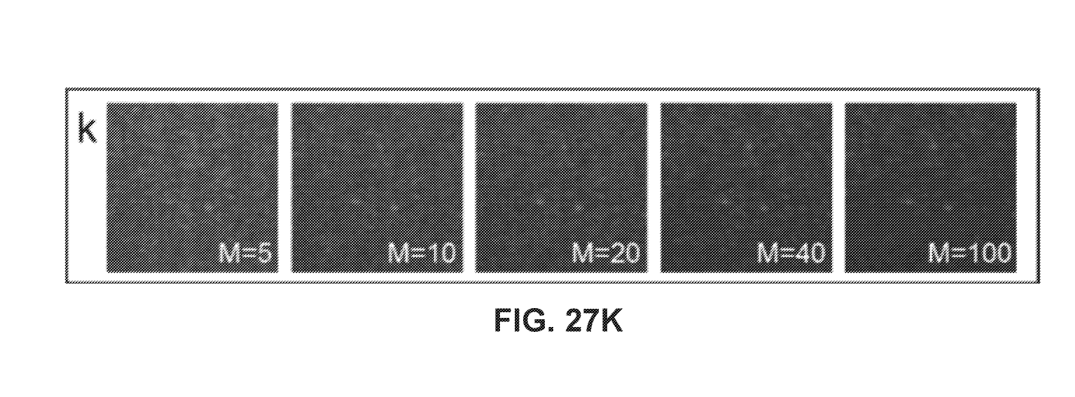

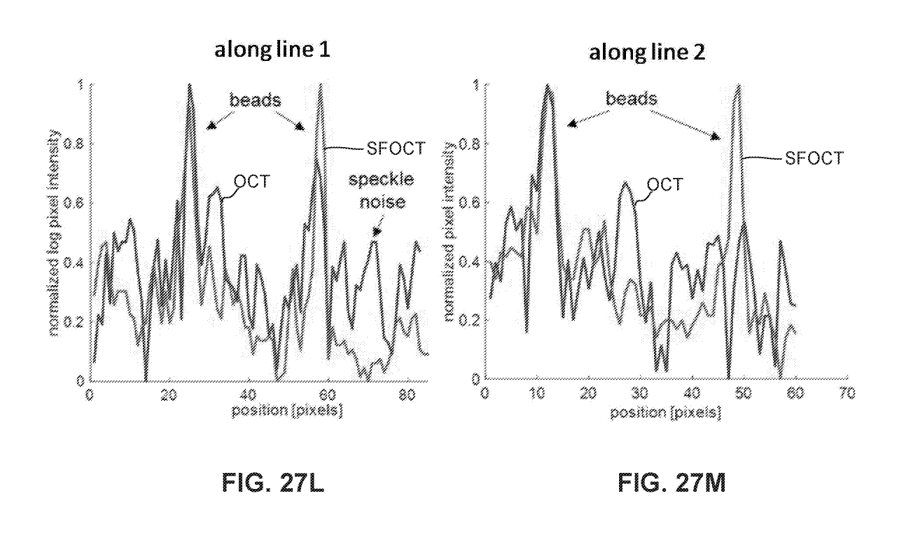

The speckle reduction achieved with SFOCT, performed by any of the systems and in accordance with any of the methods described herein, can reveal fine structures that are otherwise obscured by noise. The capability of SFOCT can be demonstrated by imaging polystyrene beads with 3 μm diameter embedded inside a GNR and agarose phantom. FIG. 27A shows a bright field microscope image of a thin slice of a GNR-agarose phantom with 3 μm diameter beads (10×). The image shows the beads sparsely dispersed in a uniform phantom, and as shown, the GNRs are too small to be visible individually. FIGS. 27B-27C show OCT and SFOCT B-scans, respectively, of an agarose phantom with TiO₂ nanopowder. The nanopowder does not disperse well in the phantom, rather it forms large clumps. These clumps are hidden within the speckle noise in the OCT image, but are revealed in the SFOCT image. FIG. 27D shows a bright field microscope image of the phantom presented in FIGS. 27B-27C, showing the shape size and distribution of the clumps in the agarose base. The comparison of SFOCT images to microscope images shows that SFOCT provides a closer representation of the true nature of the phantoms compared to OCT.

FIGS. 27E and 27I show OCT and SFOCT B-scans, respectively, of a phantom made by dispersing GNRs and 3 μm diameter polystyrene beads in an agarose gel. In the OCT image shown in FIG. 27E, many of the beads are covered by speckle noise and cannot be detected. In the SFOCT image shown in FIG. 27I, however, speckle noise is eliminated and the beads become visible, along with the random distribution of GNRs in the phantom.

FIGS. 27F-27G show close-up views of rectangular regions marked as regions Zg and Zf, respectively, in FIG. 27E. FIGS. 27J and 27K show close-up views of rectangular regions marked as regions Zk and Zj, respectively, in FIG. 20I. These marked regions include two beads that are not visible in OCT but observable in SFOCT. In the OCT image the right-most bead is completely hidden by the noise. FIG. 27K further shows that the beads are revealed when the number of averaged frames increases (averaged frames are 5, 10, 20, 40 and 100).

FIG. 27H shows a schematic of the locations of the three beads. FIGS. 20L and 20M show intensity profiles along line 1 and line 2, respectively, illustrated in FIG. 27H. While the beads are easily visible in SFOCT, in OCT, the intensity varies, which makes the beads difficult to identify.

As shown in FIGS. 27A-27M, speckle noise can be predominant in conventional OCT and consequently hides many of the beads. SFOCT, however, reveals the beads along with the distribution of the GNRs in the agarose phantom. Furthermore, FIG. 27K demonstrates the improvement in speckle reduction achieved using SFOCT as a function of averaging. The signal intensity profiles show the reduction of speckle noise and the presence of the beads identified using SFOCT. Comparison with bright field microscopy further validates that SFOCT yields more accurate representations of the structure of the sample than OCT.

Biomedical Imaging of SFOCT

One of the biomedical advantages of OCT is that it can provide noninvasive high resolution images inside living tissues. Strong speckle artifacts, however, drastically limit insight regarding fine anatomical structures. These limitations become obvious upon comparison with histological tissue sections. By removing the significant contribution of speckle noise, SFOCT is capable of rendering in vivo images that approach histological detail.

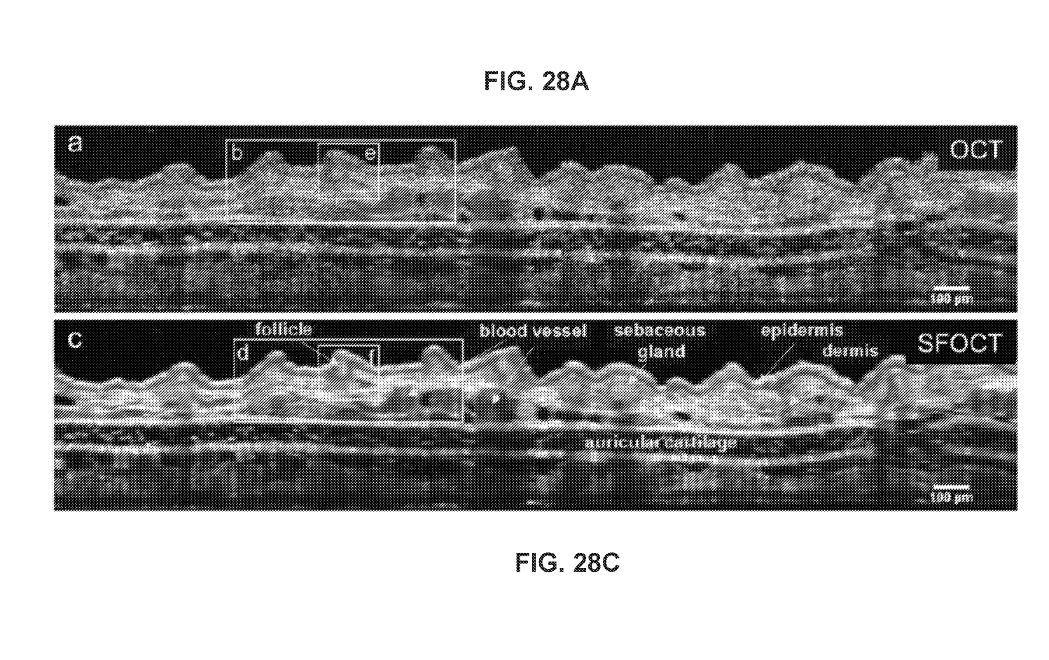

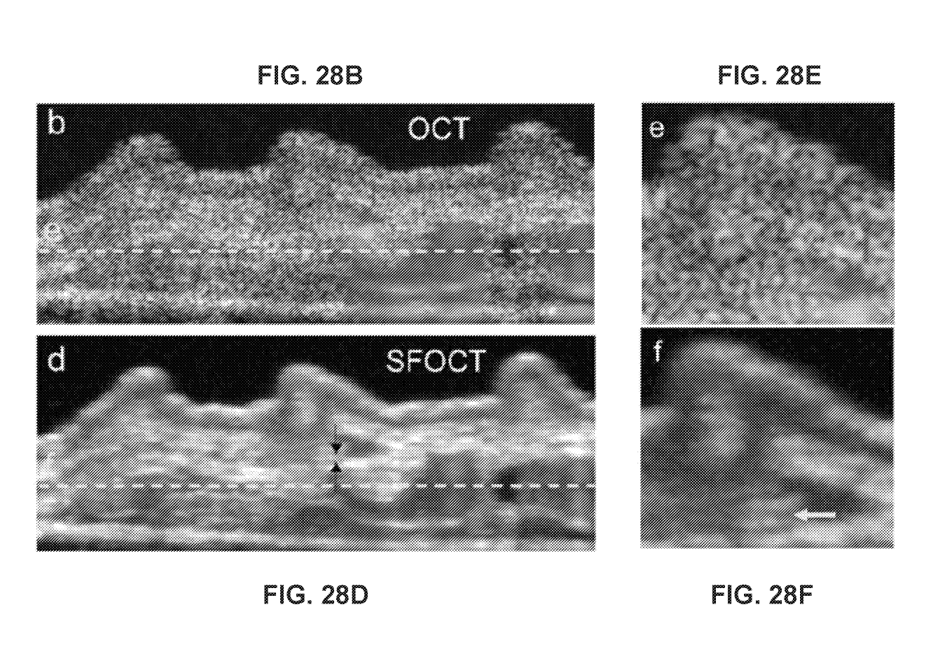

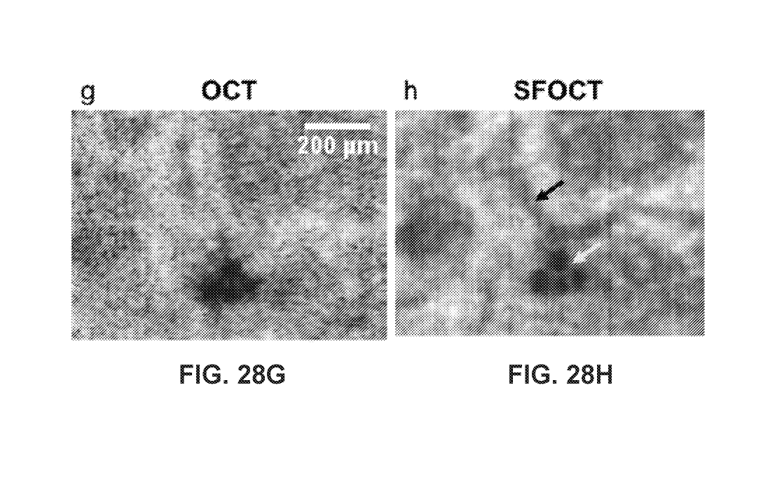

As one example, FIG. 28A shows a conventional OCT image of a mouse pinna, which includes epithelial and cartilage layers, small blood and lymph vessels, and numerous hair follicles and sebaceous glands. FIGS. 28B and 28E show close-up views of the regions marked as regions b and e, respectively, in FIG. 28A.

FIG. 28C shows a scan of the mouse pinna obtained using SFOCT in accordance with the systems and methods described herein, and in particular, with the 1500 grit diffuser as the phase scrambler. FIGS. 28D and 28F show close-up views of the regions marked as regions d and f, respectively, in FIG. 28C. The opposing arrows in FIG. 28D show an anatomical feature the size of 9 μm. Features of this small size are normally not observed in the conventional OCT images due to speckle noise. The arrow in FIG. 28F shows a dark line which is 2 μm thick, demonstrating that the intrinsic axial resolution, as defined by the broadness of the spectrum, is unharmed.

FIG. 28G shows an OCT en face scan at the depth indicated by the dashed line in FIG. 28B. FIG. 28H shows a SFOCT en face scan at the depth indicated by the dashed line in FIG. 28D, revealing lymph vessels (black arrow) and fine structures (yellow arrow). FIG. 28I is a microscope image of H&E stained mouse ear pinna at 10× magnification.

As seen from FIGS. 28A-28I, many of the structures masked by speckle noise in the OCT image become readily apparent in the SFOCT images. Speckle removal allows imaging of fine structures in B-scans as well as in en face images, indicating that SFOCT provides improvement in image quality in all three spatial dimensions. The noise in voxels (or resolution volumes) within the tissue can be much higher than that of voxels (or resolution volumes) above the tissue, showing that speckle noise can be much larger than photon shot noise and thermal noise in these measurements.

As also evident by the images and characterization presented in FIGS. 28A-28I, the quality of images of turbid media can be superior compared to that of OCT owing to the removal of speckle. The axial resolution of the system can be preserved owing to the averaging of the thickness variations in time. This can be the reason why vertical features as small as 2 μm are observed (such as the dark line in FIG. 28F).

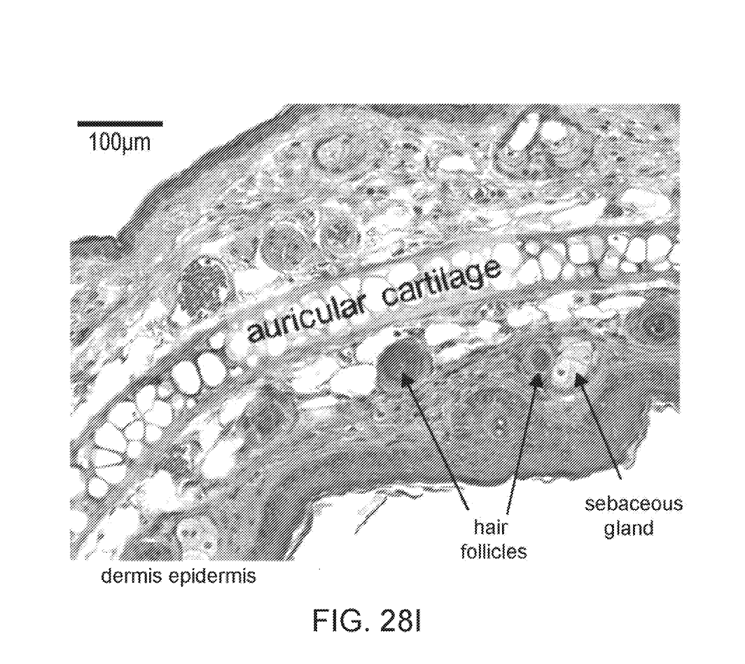

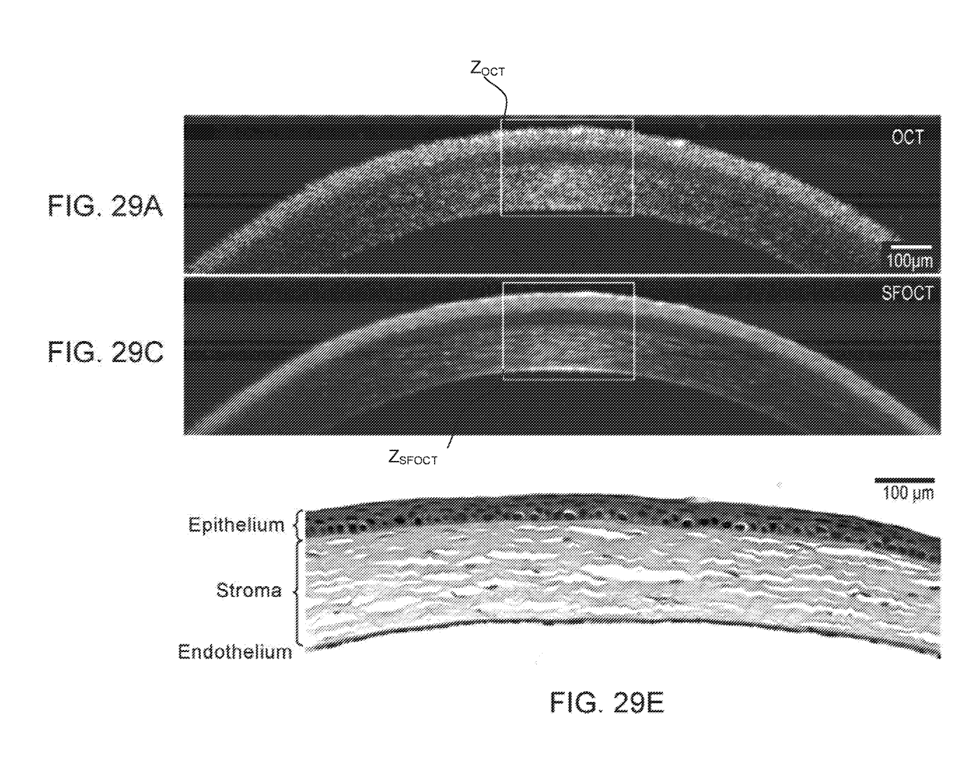

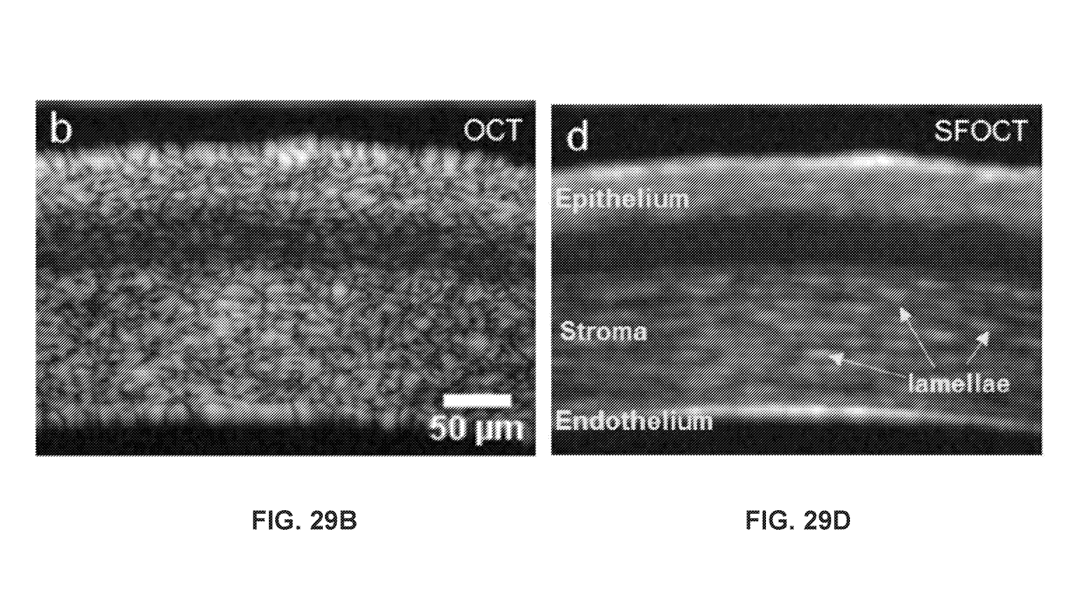

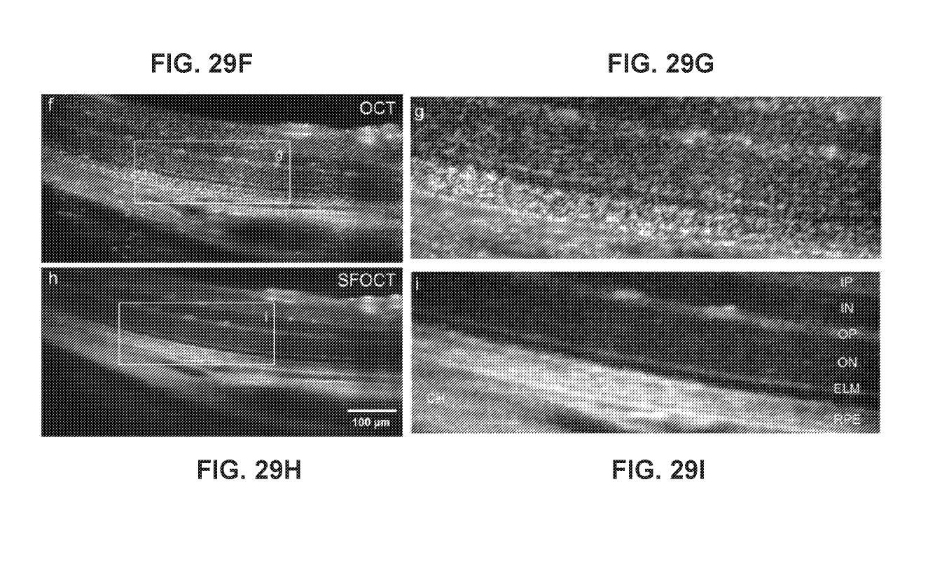

Another common clinical application of OCT is for ophthalmic imaging. FIG. 29A-29I show imaging of the mouse cornea and retina performed according both convention OCT and SFOCT using the methods and described herein. As shown, the SFOCT imaging clarifies the boundaries between the layers and reveals the cellular structure of the stroma. More specifically, FIG. 29A shows an OCT B-scan of a mouse cornea. FIG. 29B shows a close-up view on the region marked as Z_{OCT} in FIG. 29A. FIG. 29C shows a SFOCT scan of a mouse cornea and FIG. 29D shows a close-up view on the region marked as Z_{SFOCT} in FIG. 29C. FIG. 29E is a microscope image of H&E stained mouse cornea at 10× magnification.

FIG. 29F shows an OCT B-scan of a mouse retina and FIG. 29G shows a close-up view of the region marked as region g in FIG. 29F. FIG. 29H shows a SFOCT scan of a mouse retina and FIG. 29I shows a close-up view on the region marked as region h in FIG. 29H.

In FIG. 29I, various structures are identified. They include: IP, inner plexiform; IN, inner nuclear layer; OP, outer plexiform layer; ON, outer nuclear layer; ELM, external limiting membrane; RPE, retinal pigment epithelium; CH, choroid.

Using SFOCT, the lamella structure of the stroma along with clear boundaries between the other layers of the cornea can be observable. Due to speckle noise, conventional OCT is not able to show clear boundaries between layers or the structure of the stroma. FIGS. 29F-I show that the individual layers of the retina are particularly well resolved with SFOCT. For example, the outer plexiform layer and the external limiting membrane are more resolvable in SFOCT.

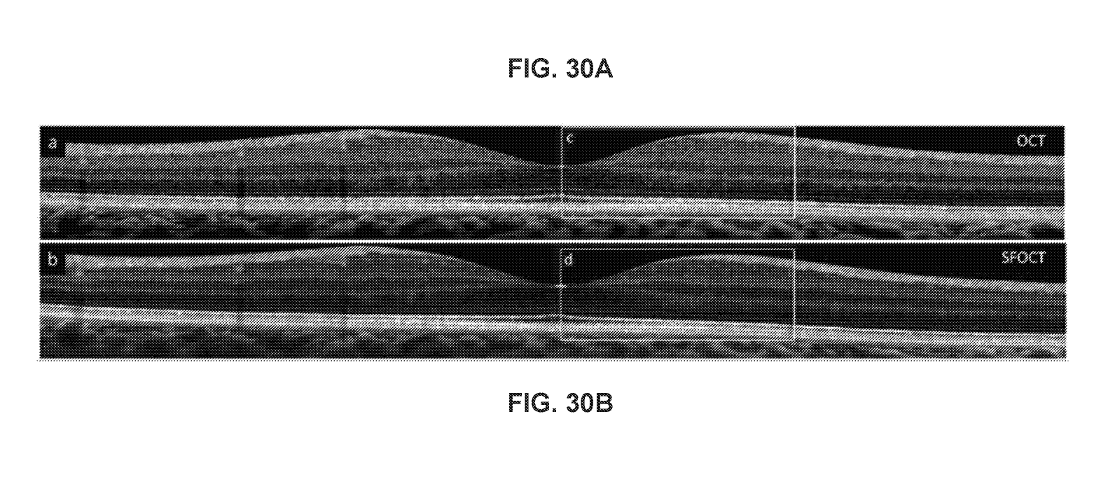

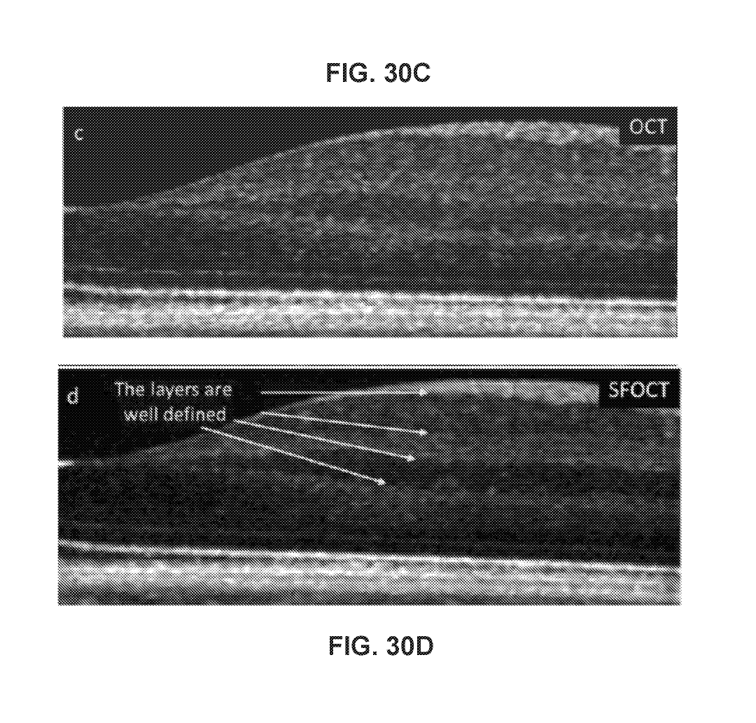

FIGS. 30A-30D show SFOCT imaging of human retina. FIG. 30A shows an OCT B-scan (cross-section) of a retina. FIG. 30B shows a SFOCT scan of the retina. FIGS. 30C and 30D show close-up views on the region marked in FIGS. 30A and 30B, respectively. The SFOCT images of the retina show a clearer differentiation between the retinal layers.

In the retinal systems, the power of the light source is normally limited to a certain safety level (e.g., due to ANSI safety guidelines). A finer grit diffuser produced by lapping a 1500 grit diffuser with 3 μm particles is used as the phase scrambler in the SFOCT system for this imaging. As discussed above, the finer diffuser can produce images with a higher signal to noise ratio but may also reduce less speckle noise. Although speckle noise may still be present in the retina images, the retinal layers are better defined in SFOCT when compared to conventional OCT.

Other than ophthalmic applications, OCT is gaining popularity in dermatology owing to its potential for doing noninvasive biopsy. Speckle noise, however, may prevent seeing clear boundaries between anatomical objects and limits the visibility and identification of small or low contrast structures. Speckle elimination can enhance the diagnostic capabilities of OCT.

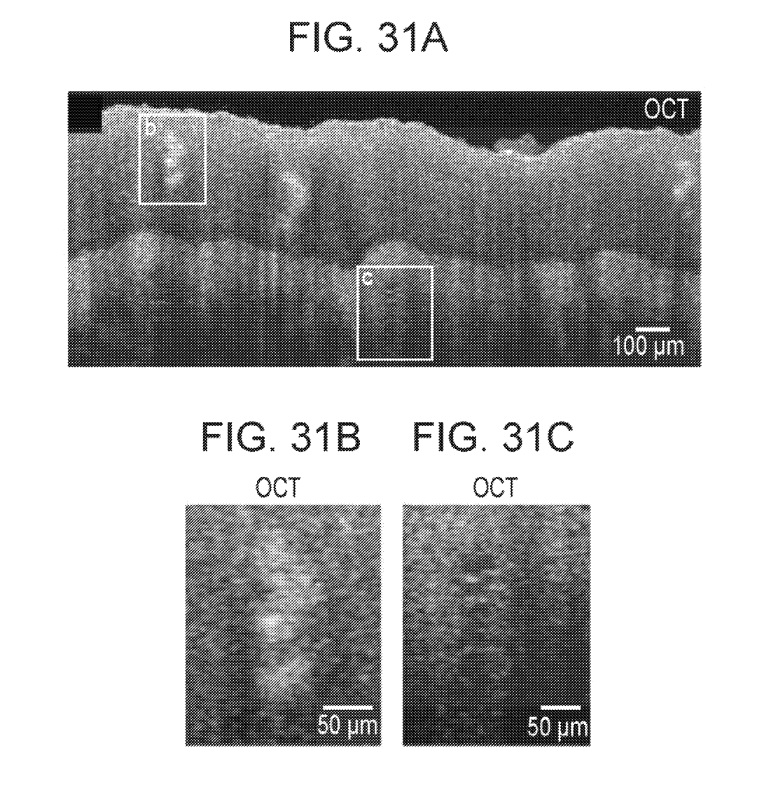

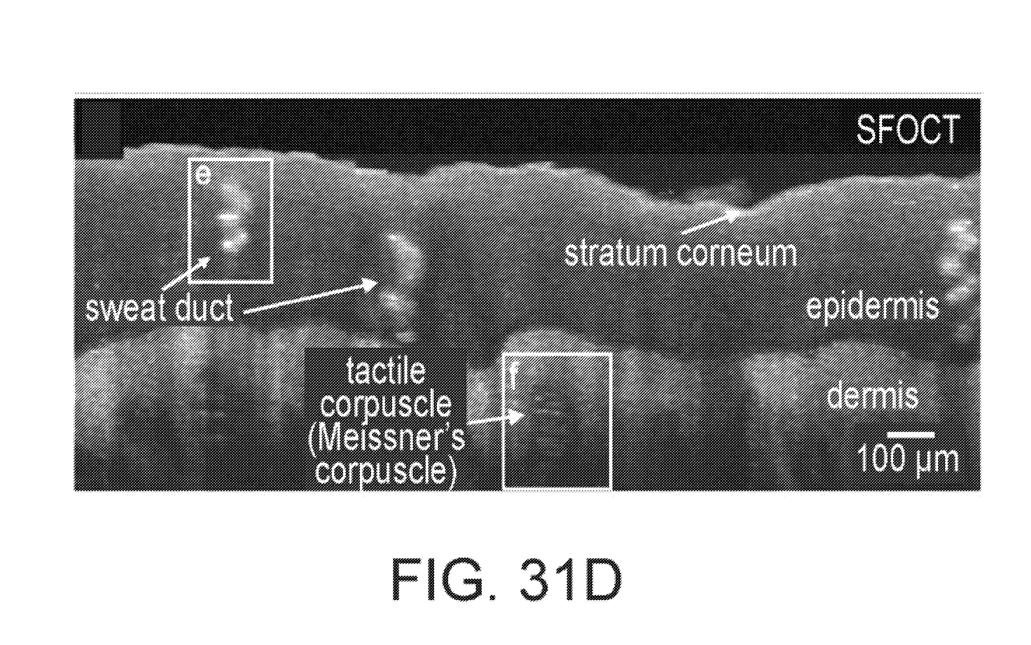

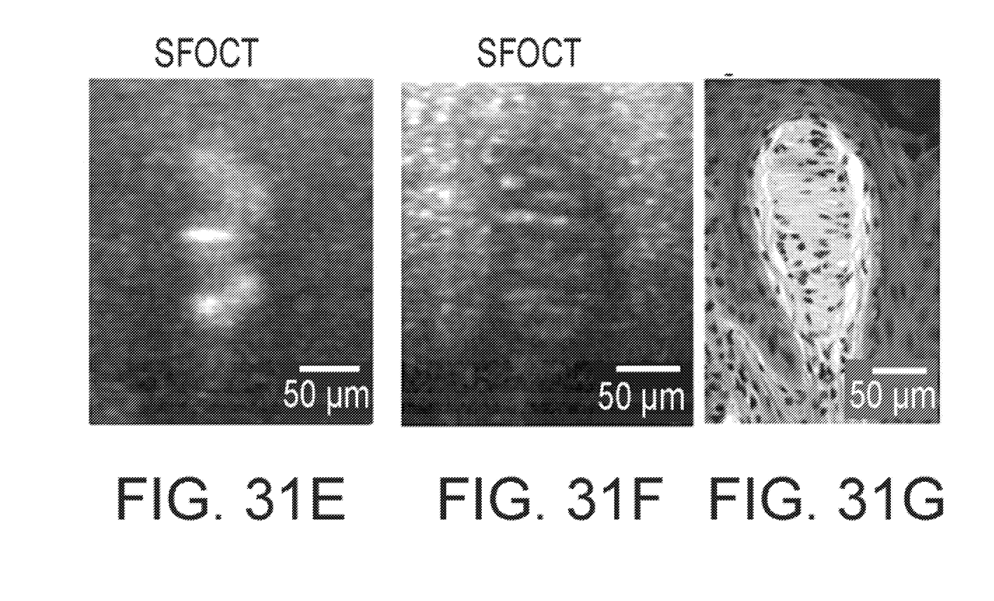

FIGS. 31A-31G show OCT and SFOCT imaging of intact human fingertip skin and reveal fine structures such as the sweat ducts and the tactile corpuscle. More specifically, FIG. 31A shows an OCT B-scan of a fingertip. FIG. 31B shows a close-up view on the sweat duct marked in FIG. 31A. FIG. 31C shows a close-up view on the tactile corpuscle marked in FIG. 31A.

FIG. 31D shows a SFOCT scan of a fingertip. FIG. 31E shows a close-up view on the sweat duct marked in FIG. 31D. FIG. 31F shows a close-up view on the tactile corpuscle marked in FIG. 31D. FIG. 31G is a microscope image of H&E stained tactile corpuscle (Courtesy of the Dept. of Anatomy, UCSF School of Medicine).

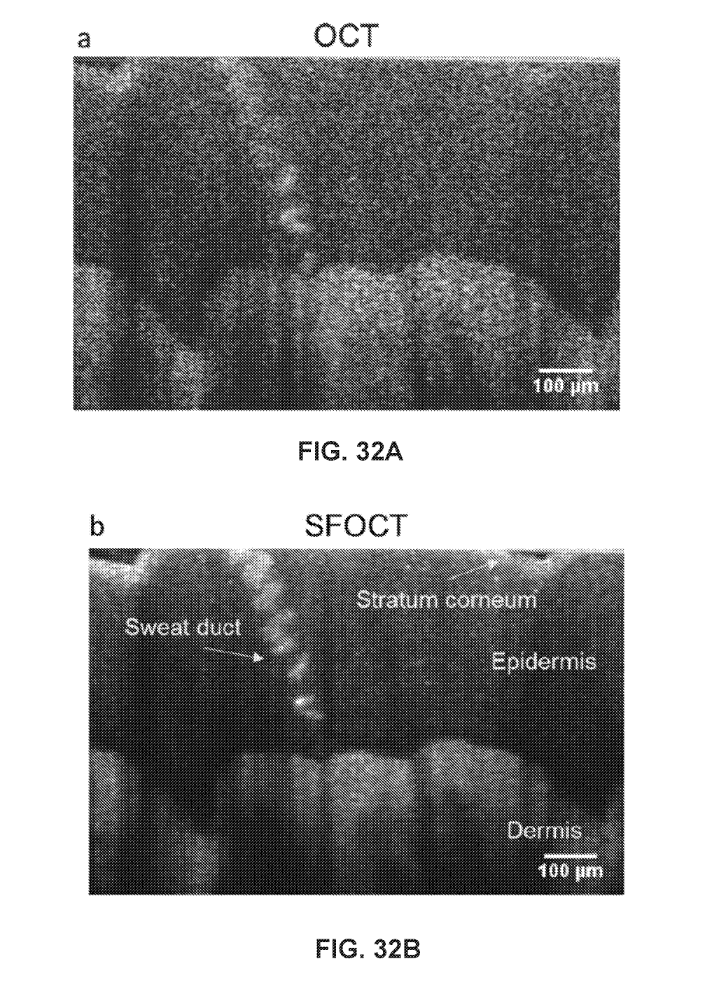

FIG. 32A shows an OCT B-scan of a fingertip showing a sweat duct. FIG. 32B shows a SFOCT B-scan of a similar region, showing the sweat duct in greater detail, along with a better view of the layers in the skin.

As seen from FIGS. 31A-24G and FIG. 32A-32B, SFOCT can reduce speckle noise significantly and is able to show the fine structures of the sweat duct and the tactile corpuscle. SFOCT can be particularly helpful in identifying the boundaries between the corpuscle and the surrounding dermis. Much like the images of the mouse cornea, images of the fingertip using SFOCT reveal the cellular structure of the tactile corpuscle, proving that it can remove speckle noise without compromising resolution. This example suggests that SFOCT could be used in noninvasive dermatological studies in humans and produce images that approach the quality of histology.

Other Speckle Noise Processing Techniques

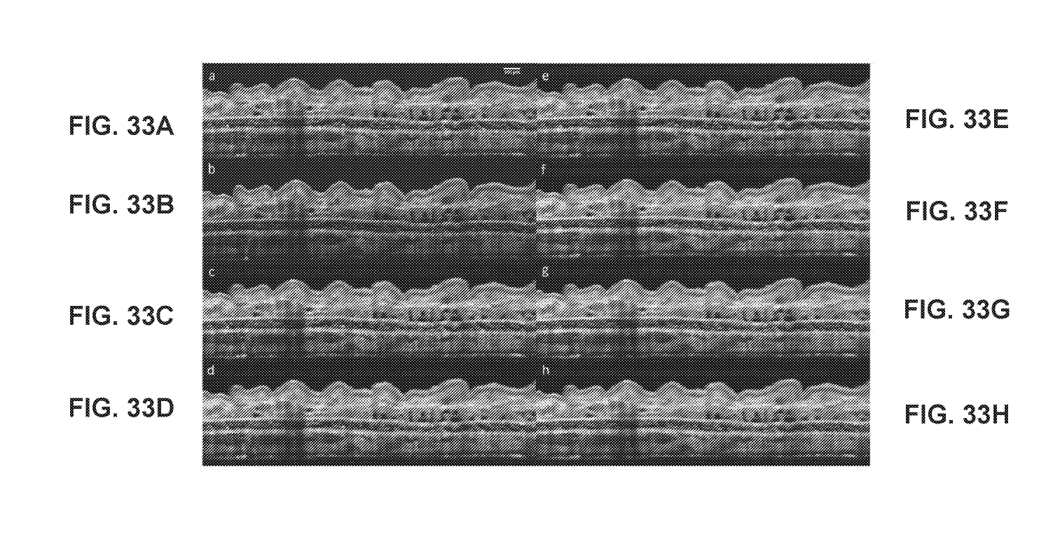

Speckle noise may be reduced by other techniques, such as spatial compounding, adaptive Wiener filtering, symmetric nearest-neighbor, and hybrid median filter. These cases can be used in combination with SFOCT techniques. For example, FIGS. 33A-33H show speckle noise reduction using SFOCT, spatial compounding, and 3D smoothing techniques. FIG. 33A shows an OCT scan sampled every 4 μm in both lateral directions, with 20 B-scan averages. The image can be created by averaging 2 adjacent frames (to improves SNR, averaging is of linear-scale images) and later resampled to obtain a voxel size of 2 μm in all three directions. Averaging 2 scans, which span 4 μm, normally does not reduce speckle because the averaged scans are inside the PSF. FIG. 33B shows a SFOCT scan, with the 1500 grit diffuser as a phase scrambler, using the same acquisition parameters and post processing as in FIG. 33A.

FIGS. 33C and 33D show images produced from three-dimensional smoothing of the OCT volume described in FIG. 33A. Smoothing can be done on the linear-scale image after resampling using Matlab's smooth3 function with a Gaussian kernel in a square window with a size of 11 pixels. The standard deviation of the Gaussian is 0.95 in FIG. 33C and 1.25 in FIG. 33D. FIG. 33E-33H show averaging and processing of the OCT volume as described in FIG. 33A, using 4, 7, 9, and 13 frames, respectively (spanning 12, 24, 32, and 48 μm).

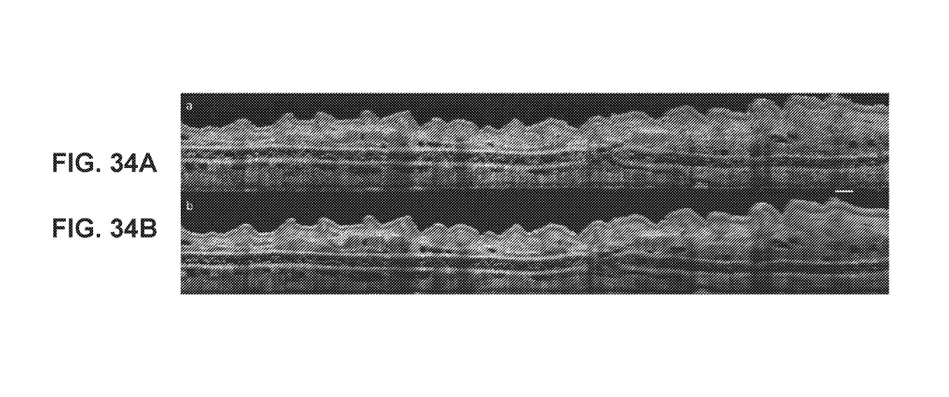

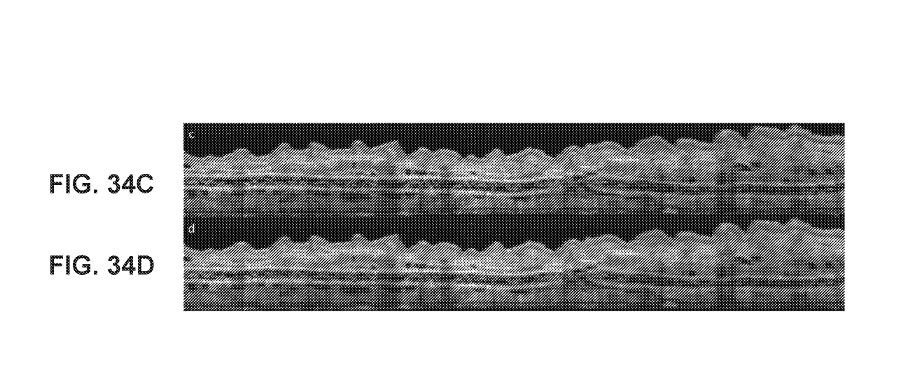

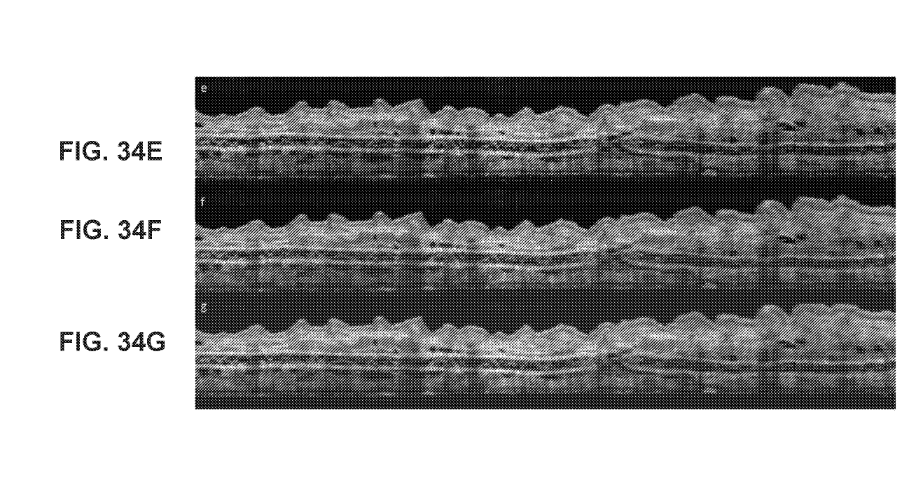

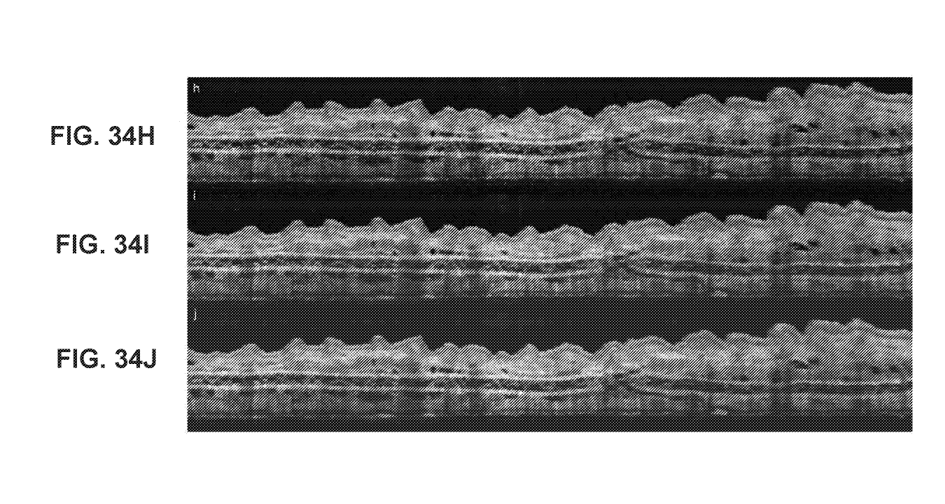

FIGS. 34A-34J show reduction of speckle noise using SFOCT and digital filtering methods. The digital filters can be applied on a logarithmic scale OCT image. FIG. 34A shows an OCT image of a mouse pinna. The pixel size is 2×2 μm. FIG. 27B shows a SFOCT image at a similar location. FIGS. 34C-34D show the OCT image after application of an adaptive Wiener filter of size 5 and 7 pixels, respectively. FIGS. 34E-34G show the OCT image after application of a hybrid median filter (HMF) of size 5, 7 and 9 pixels, respectively. FIGS. 34H-34J show the OCT image after application of a symmetric nearest-neighbor filter (SNN) of size 5, 7 and 9 pixels, respectively.

Techniques that are disclosed in this application use optical coherence tomography (OCT) as an illustrating and non-limiting example. Techniques can be implemented in all types of OCT, including time-domain OCT, swept source OCT, and spectral domain OCT. In addition, techniques disclosed herein can also be applied in any other coherence imaging techniques, such as holography, interference based profilometer, and coherent imaging and microscopy.

Additional Applications

The reduction of speckle noise performed in accordance with the systems and methods described herein not only reduces noise in visualizing the structure of the sample, but also reduces noise when analyzing the spectral characteristics of a sample when performing spectral analysis. Spectral analysis is performed to visualize the spectrum of scattered light from a sample, in addition to the location of the scatterer, and can reveal, for example, amount of blood oxygenation and a location of a contrast agent. Speckle noise, which is caused by interference between coherently scattered light, has spectral components therein. Thus, speckle noise is wavelength dependent, and therefore, it creates noise in the spectral analysis of the sample.

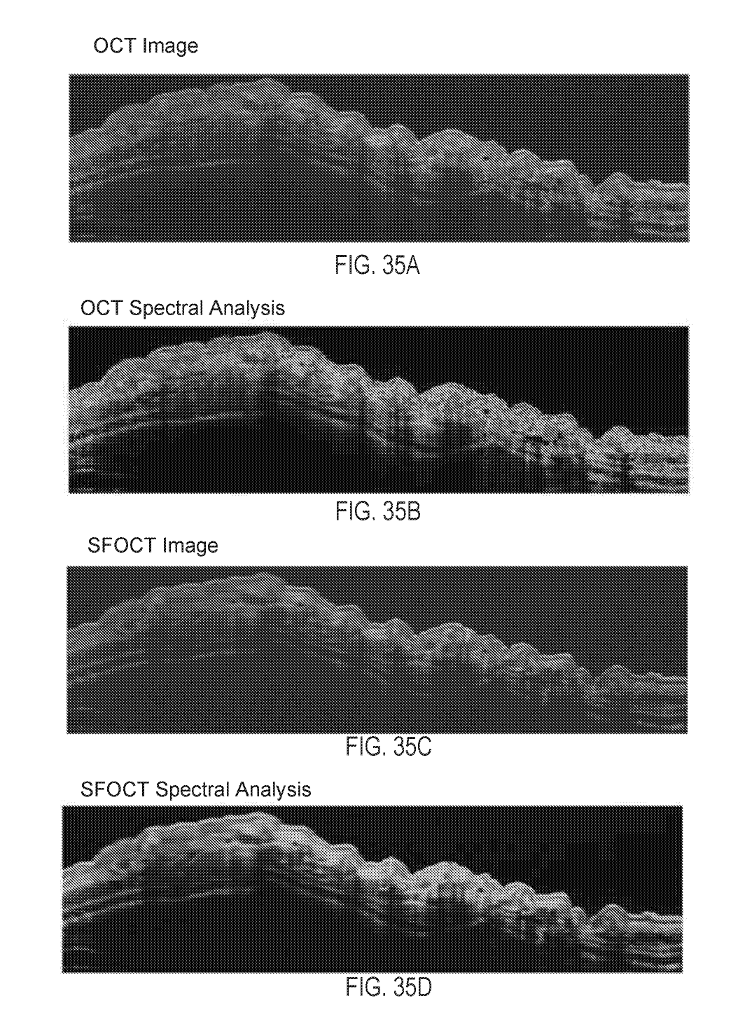

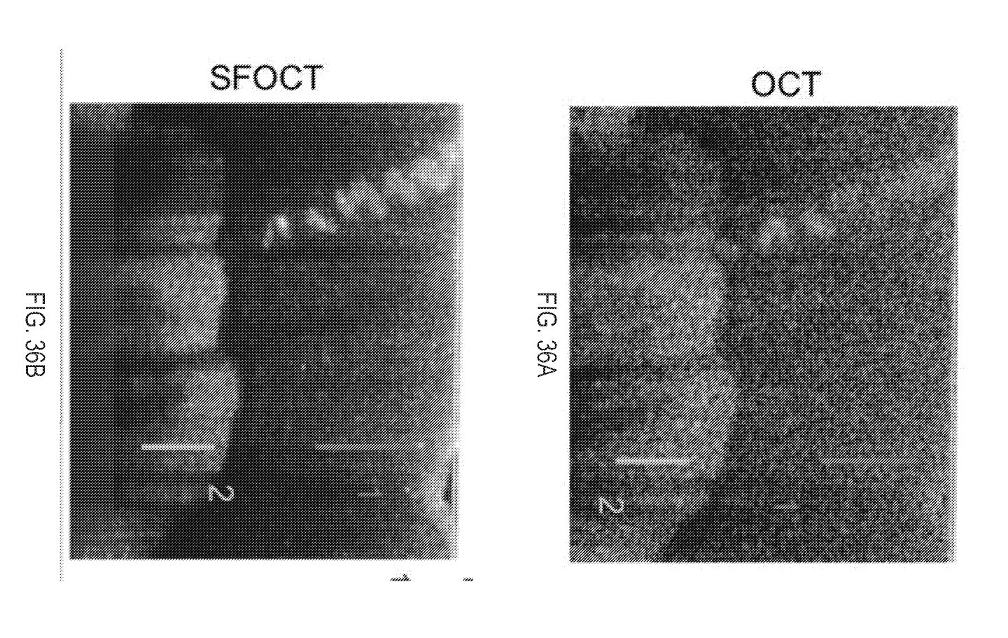

To evaluate the impact of the SFOCT systems and methods described herein on spectral analysis, a sample as imaged using both conventional OCT and SFOCT in accordance with the methods described herein, and spectral analysis was then performed. The spectral analysis can be performed using any suitable method, such as, for example, the method disclosed in “Contrast-enhanced optical coherence tomography with picomolar sensitivity for functional in vivo imaging,” by O. Liba et al., Sci. Rep., vol. 6, p. 23337, March 2016. The results of the spectral analysis for each frame were then averaged. Each frame has a different spectral-speckle pattern because of the different phases projected by the phase scrambler (or diffuser). After averaging, the spectral-speckle noise is considerably reduced. The results are shown in FIGS. 35A-35D. Particularly, FIGS. 35A-35B show an image and a spectral analysis image, respectively, of a tumor in an ear pinna of a mouse based on scans obtained with standard OCT. FIG. 35C-35D show an image and a spectral analysis image, respectively, based on scans obtained with SFOCT according to the embodiments described herein. As shown, employing SFOCT for spectral noise reduction allows for a more accurate characterization of the spectral components of the sample, in addition to a more accurate visualization of the structure.

In some embodiments, a method includes transmitting a light beam to illuminate a resolution volume associated with a sample. The light beam is spatially modulating to introduce a first local phase change to a first portion of the light beam and to introduce a second local phase change to a second portion of the light beam. The second local phase change different than the first local phase change. The light beam is then temporally modulating between successive image capture events to produce a first speckle pattern at a first time in a first image associated with the resolution volume and to produce a second speckle pattern at a second time in a second image associated with the resolution volume. The second speckle pattern is different than the first speckle pattern. A spectral analysis is then performed on a series of images, including those with the first speckle pattern with the second speckle pattern. The first speckle pattern and the second speckle pattern are averaged to reduce speckle noise in a third image associated with the resolution volume. The third image can be, for example, associated with a spectral analysis.

In some embodiments, the SFOCT apparatus and method described herein can be applied to the measurement of an absorption profile of a sample. In such embodiments, the reduction and/or elimination of the speckle noise produces a more accurate measurement of the absorption profile than measurements taken using convention OCT methods. In particular, studies have shown that using OCT to measure the local attenuation coefficient in tissue can provide diagnostic information, such as the detection of tumor margins. The attenuation coefficient of a sample may be calculated by fitting the optical coherence tomography signal intensity to a function that includes the effects of the Beer-Lambert law (an exponential function), the confocal function, OCT roll-off and multiple scattering.

In some embodiments, a method includes applying SFOCT to the measurement of local attenuation coefficients. In some embodiments, a method includes varying a local phase of a sample light beam, as described herein, between successive images to reduce speckle noise. The method further includes evaluating the signal intensity of the images to determine a boundary of a structure within the sample. As described herein, by applying the SFOCT methods described herein to remove and/or reduce the speckle noise, a more precise fit can be produced.

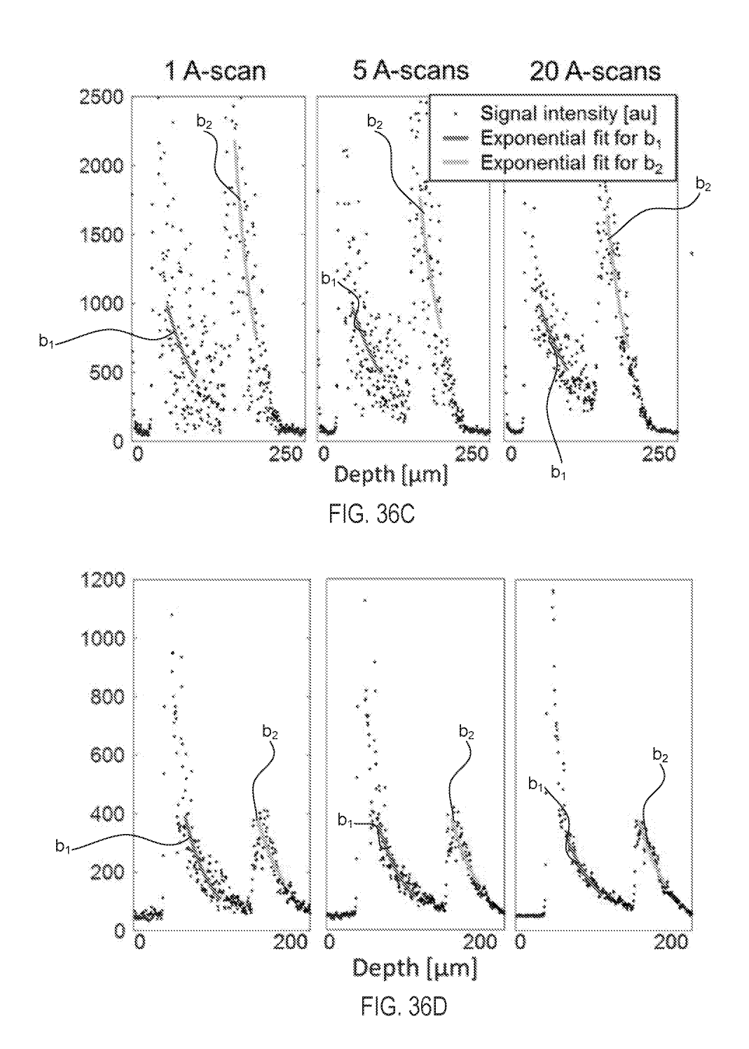

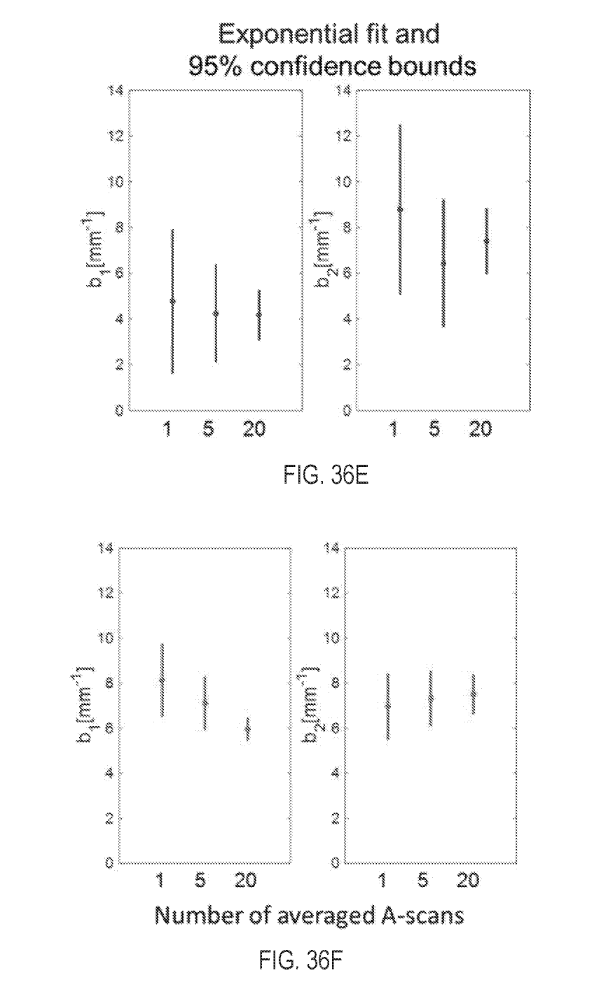

To compare the precision of the fit between conventional OCT and SFOCT, an exponential fit to the signal intensity was performed based on scans conducted using both techniques. The simplified model described herein assumes that tissue attenuation, governed by the Beer-Lambert law, is the most dominant factor in the decrease of signal intensity as a function of depth. The precision of the fit between images of a fingertip produced using conventional OCT and SFOCT according to the methods described herein was compared. (see e.g., FIGS. 36A-F described below). The precision of the fit was determined by the 95% confidence bounds.

The Beer-Lambert law is given by the following expression:

i(z)∝\sqrt{exp(−2μz)}=exp(−bz) (7)

in which i (z) is the depth dependent OCT or SFOCT signal intensity and μ is the attenuation coefficient. b is the exponential coefficient that we are attempting to find by fitting the signal to an exponential function.

FIGS. 36A-36B show images of a fingertip taken using convention OCT methods and SFOCT methods according to an embodiment, respectively. In particular, these images are scans of a fingertip showing a sweat duct. The lines labeled as 1 and 2 indicate the regions used for the calculation of the exponential fit parameters, b₁ and b₂, set forth in the Beer-Lambert law equation given above (Equation 7).

FIGS. 36C-36D are graphs of the pixel values (in a linear scale) representing the intensity as a function of depth from the images shown in FIGS. 36A and 36B, respectively. FIGS. 36C and 36D also include the exponential curve fit. The curve fit calculation was performed for both the OCT and SFOCT scans based on pixel values of one A-scan, and based on intensity averaged values of 5 and 20 adjacent A-scans. FIGS. 36E-36F are plots showing the calculated exponential coefficients (b₁ and b₂) with error bars representing the 95% confidence bounds, based on the images shown in FIGS. 36A and 36B, respectively. The tighter bounds indicate a more precise measurement. As shown, in the measurement taken using the SFOCT methods, the exponential coefficient is more precise in all cases because of the reduced speckle noise. To achieve a higher precision with OCT, adjacent A-scans should be averaged, thereby reducing the spatial resolution of the measurement.

In some embodiments, a method can include changing the local phase of a light beam during optical coherence tomography to reduce and/or eliminate speckle noise to improve the automated segmentation of structures in the imaged tissue. For example, when analyzing the retina, segmentation is used to determine the thickness of the retinal layers and diagnose numerous conditions. In some embodiments, reducing speckle noise using the SFOCT methods and systems described herein can improve segmentation results.

To evaluate the effects of SFOCT on segmentation algorithms, segmentation of mouse retina was used as a test case. The external limiting membrane (ELM) was analyzed, because this thin layer is known to be one of the most difficult layers to detect in retina imaging using conventional OCT methods. Particularly, imaging of this thin layer often results in poor signal-to-noise ratio (SNR). Many known retina layer segmentation algorithms use graph cut segmentation. Interestingly, a recent study examined the effect of SNR of the image on segmentation error rate, and found that at a border case in which the SNR is 2, the expected error rate would be 15.8%. In many instances, this level of error is considered as practically sufficient for many segmentation problems. Of course, the higher the SNR, the lower the resulting error rate. Thus, increasing the SNR using the methods and systems described herein can provide improved segmentation.

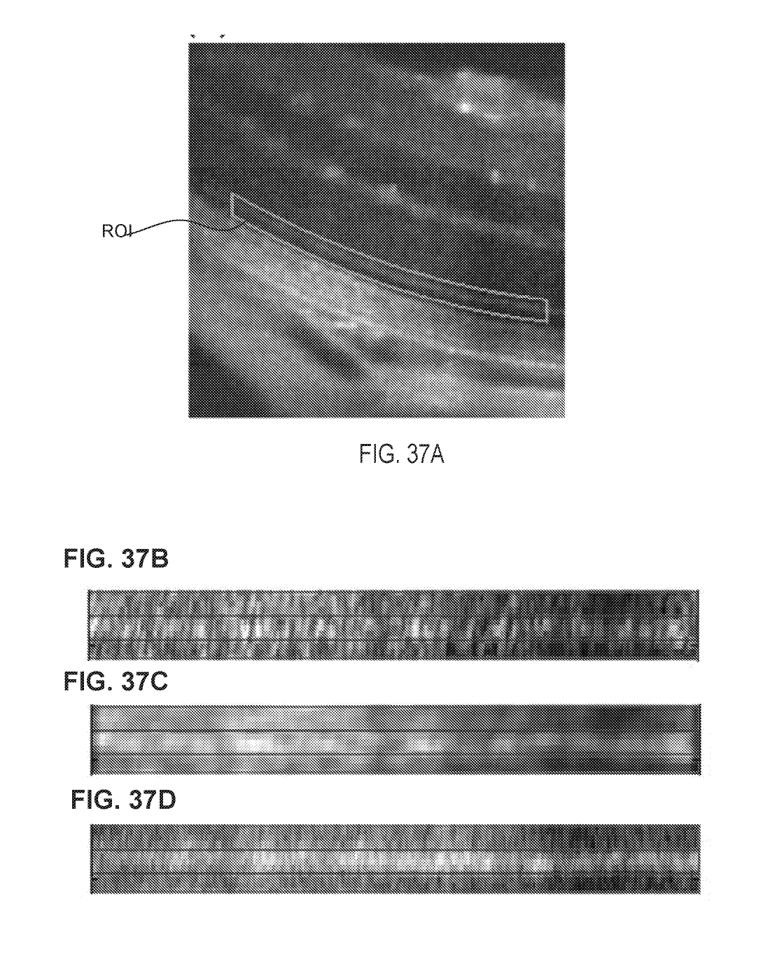

To test this method, a mouse retina was imaged using convention OCT methods and SFOCT methods according to an embodiment. A region of interest (ROI) was selected around the ELM (See, FIG. 37A, which shows the image taken using SFOCT methods). The ROI was flattened and the SNR was examined for the conventional OCT image (FIG. 37B), and for the convention OCT image with lateral smoothing of 12 pixels (24 μm) (FIG. 37C). The SNR was also examined for the SFOCT image without any lateral smoothing (FIG. 37D). SNR was computed according to the formula:

$$\begin{matrix}S N R = \frac{{( {μ}_{FG} - {μ}_{BG} )}^{2}}{\frac{1}{2} \frac{1}{\vert FG \vert} {\sum }_{p ∈ FG} {( {I}_{p} - {μ}_{FG} )}^{2} + \frac{1}{2} \frac{1}{\vert BG \vert} {\sum }_{p ∈ BG} {( {I}_{p} - {μ}_{BG} )}^{2}} & ( 8 ) \\ {μ}_{FG} = \frac{1}{\vert FG \vert} = {\sum }_{p ∈ FG} {I}_{p} {μ}_{BG} = \frac{1}{\vert BG \vert} {\sum }_{p ∈ BG} {I}_{p} & ( 9 )\end{matrix}$$

Where Iₚ is the image log intensity for pixel p balanced such that each column has the same mean intensity, FG are pixels within the foreground area, BG are pixels within the background area.

The signal to noise ratios were measured as being 0.76 for the OCT image, 2.56 for OCT with 24 μm lateral smoothing, and 3.30 for SFOCT with no lateral smoothing. These measurements imply that it is possible to reach similar levels of SNR (and similar segmentation quality as a result) with both OCT and SFOCT only if a significant lateral smoothing is applied to the OCT image (FIG. 37C). However, OCT with lateral smoothing loses the ability to detect small (less than 24 μm in our example) displacements in the ELM. Such a loss in fine feature resolution hinders clinical OCT applications for early detection of diseases. In contrast, SFOCT (FIG. 37D) achieves equal or better SNR improvements without sacrificing spatial resolution. Indeed, conventional segmentation algorithms that perform significant lateral smoothing perform well in cases of healthy patients where the retinal layers are smooth and well-defined. However, it is the small and subtle changes to the retinal layers, indications of early stages of disease that these conventional algorithms cannot detect due to poor SNR and/or significant lateral smoothing.

We further quantified SFOCT improvements to segmentation quality. One common way to measure segmentation quality is to compute the mean absolute difference (MAD) between an automated algorithm and a manual segmentation (human eye) in comparison to the MAD of two manual segmentations. In other words, MAD between two manual segmentations is considered as a reference point when measuring segmentation quality. Therefore, to assess the possible improvement in segmentation quality resulting from SFOCT, we asked 3 study-blinded, unbiased subjects to segment the ELM within the given ROI. The subjects returned mean MAD of 0.674 pixels for OCT images, 0.339 pixels for OCT with lateral smoothing, and 0.341 pixels for SFOCT. The significant difference between segmentation of OCT and SFOCT images (t-test p≤0.02) provides evidence that, in this case, a future automated SFOCT segmentation could be significantly more accurate than an OCT image segmentation algorithm. No significant difference in MAD was observed when comparing OCT with lateral smoothing and SFOCT

Additional Methods

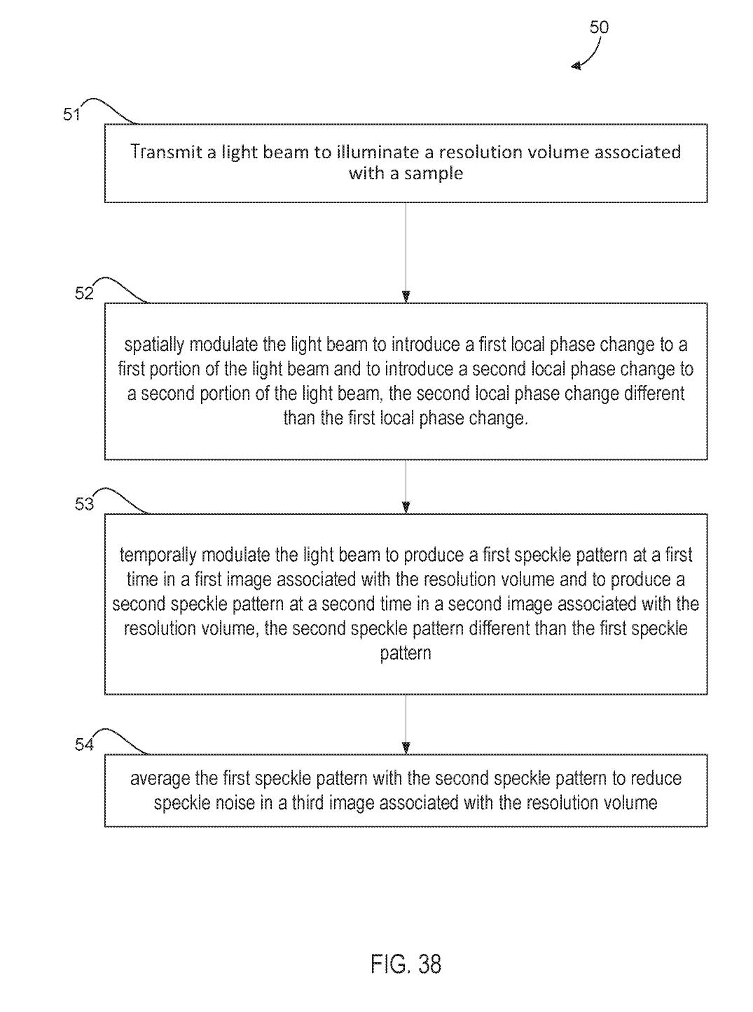

As described above, the systems and methods described herein can be used in any suitable application. Moreover, any of the above-described applications can employ any of the methods of scanning, average and phase scrambling described herein. For example, any of the systems and applications described herein can be performed using the method of optical coherence tomography shown by the flow chart in FIG. 38. As shown, the method 50 includes transmitting a light beam to illuminate a resolution volume associated with a sample, at 51. The light beam can be transmitted using any of the systems described herein, such as the system 100 or the system 800. The light beam is spatially modulated to introduce a first local phase change to a first portion of the light beam and to introduce a second local phase change to a second portion of the light beam, at 52. The second local phase change is different than the first local phase change. The spatial modulation can be performed using any suitable device or system described herein, such as any of the phase scramblers described herein. For example, in some embodiments, the spatial modulation can be performed using a ground glass diffuser, as described above.

The light beam is temporally modulated to produce a first speckle pattern at a first time in a first image associated with the resolution volume and to produce a second speckle pattern at a second time in a second image associated with the resolution volume, at 53. The second speckle pattern is different than the first speckle pattern. The temporal modulation can be performed using any suitable device or system described herein, such as any of the phase scramblers described herein. For example, in some embodiments, the temporal modulation can be performed by moving a phase scrambler within a light path between successive image capture events.

The method further includes averaging the first speckle pattern with the second speckle pattern to reduce speckle noise in a third image associated with the resolution volume, at 54.

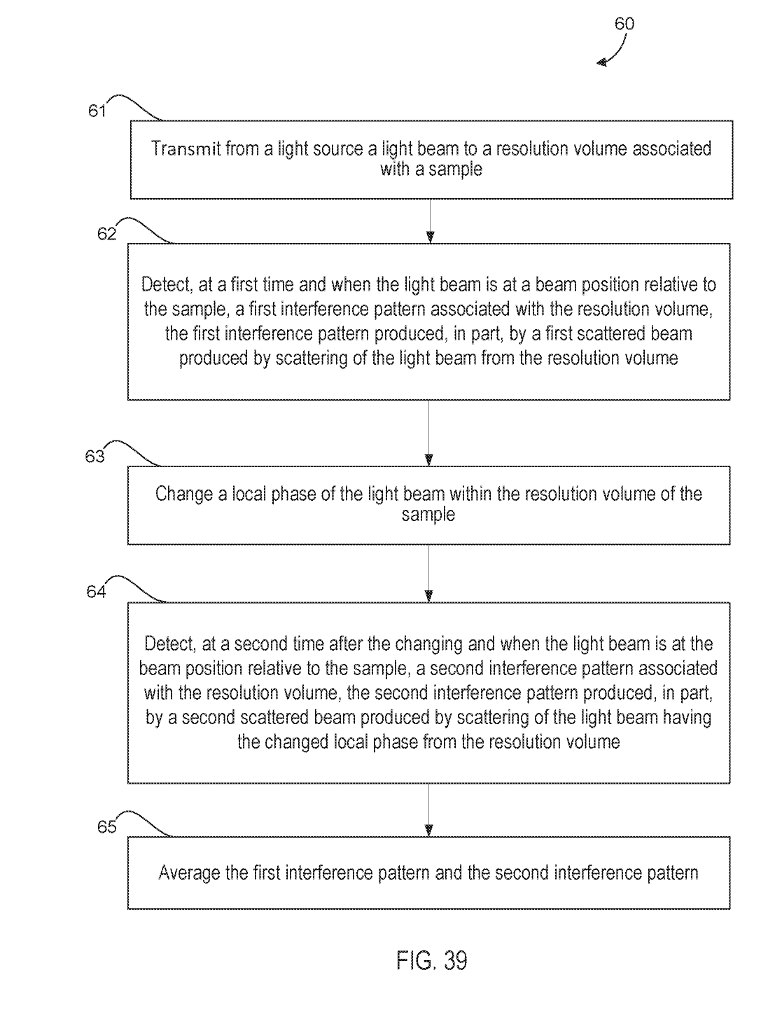

As another example, any of the systems and applications described herein can be performed using the method of optical coherence tomography shown by the flow chart in FIG. 39. As shown, the method 60 includes transmitting from a light source a light beam to a resolution volume associated with a sample, at 61. The light beam can be transmitted using any of the systems described herein, such as the system 100 or the system 800. A first interference pattern associated with the resolution volume is detected at a first time and when the light beam is at a beam position relative to the sample, at 62. The first interference pattern is produced, in part, by a first scattered beam produced by scattering of the light beam from the resolution volume.

A local phase of the light beam within the resolution volume of the sample is changed, at 63. The local phase change can be produced using any suitable device or system described herein, such as any of the phase scramblers described herein. For example, in some embodiments, the spatial modulation can be performed using a ground glass diffuser, as described above.

The method further includes detecting, at a second time after the changing and when the light beam is at the beam position relative to the sample, a second interference pattern associated with the resolution volume, at 64. The second interference pattern is produced, in part, by a second scattered beam produced by scattering of the light beam having the changed local phase from the resolution volume. The method further includes averaging the first interference pattern and the second interference pattern, at 65.

While various inventive embodiments have been described and illustrated herein, a variety of other means and/or structures for performing the function and/or obtaining the results and/or one or more of the advantages described herein. More generally, all parameters, dimensions, materials, and configurations described herein are meant to be examples and that the actual parameters, dimensions, materials, and/or configurations will depend upon the specific application or applications for which the embodiment(s) is/are used. Many equivalents to the specific embodiments described herein are possible. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The above-described embodiments can be implemented in any of numerous ways. For example, embodiments of designing and making the technology disclosed herein may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes (outlined herein) may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various disclosed concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure. The computer readable medium or media can be transportable, such that the program or programs stored thereon can be loaded onto one or more different computers or other processors to implement various aspects of the disclosure.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the disclosure need not reside on a single computer or processor, but may be distributed in a modular fashion amongst a number of different computers or processors to implement various aspects of the disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. Any suitable mechanism, however, may be used to establish a relationship between information in fields of a data structure, including through the use of pointers, tags or other mechanisms that establish relationship between data elements.

Also, various disclosed concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1-2. (canceled)

3. The apparatus of claim 11, wherein the controller includes an actuator to move the phase scrambler within the path of the second portion of the spatially coherent light beam.

4-10. (canceled)

11. An apparatus, comprising:
a light splitter to receive a spatially coherent light beam, the light splitter directing a first portion of the spatially coherent light beam to a reference arm and a second portion of the spatially coherent light beam to a sample arm, the sample arm including:
a phase scrambler at least partially in a path of the second portion of the spatially coherent light beam, the phase scrambler configured to produce a sample light beam having a spatially variable phase; and
a controller, operably coupled to the phase scrambler, to change the spatially variable phase of the sample light beam; and
a detector, in optical communication with the reference arm and the sample arm, to detect an interference pattern produced by interference of the first portion of the spatially coherent light beam propagated through the reference arm and a scattered beam produced by scattering of the sample light beam by a sample propagated through the sample arm,
wherein the detector is configured to detect the interference pattern at a first rate and the controller is configured to change the spatially variable phase of the sample light beam at a second rate greater than the first rate.

12-21. (canceled)

22. An apparatus comprising:
a light source to produce a spatially coherent light;
a light splitter, in optical communication with the light source, to split the spatially coherent light into a first beam and a second beam;
a scanner, in optical communication with the light splitter, to scan the second beam across at least a portion of a sample at a first speed to produce a scattered beam scattered by the sample;
a detector, in optical communication with the light splitter, to detect an interference between the first beam and the scattered beam;
a phase scrambler, disposed within a Rayleigh range of an image plane of a lens, to modulate a local phase of the second beam, an image of the sample at the image plane having a first magnification with respect to the sample; and
an actuator to move the phase scrambler in a direction substantially orthogonal to an optical axis of the phase scrambler at a second speed no less than a product of the first magnification and the first speed.

23. The apparatus of claim 22, wherein the spatially coherent light has a temporal coherence length of less than 10 μm.

24-26. (canceled)

27. A method of coherence tomography, comprising:
transmitting from a light source a light beam to a resolution volume associated with a sample;
detecting, at a first time and when the light beam is at a beam position relative to the sample, a first interference pattern associated with the resolution volume, the first interference pattern produced, in part, by a first scattered beam produced by scattering of the light beam from the resolution volume;
changing a local phase of the light beam within the resolution volume of the sample;
detecting, at a second time after the changing and when the light beam is at the beam position relative to the sample, a second interference pattern associated with the resolution volume, the second interference pattern produced, in part, by a second scattered beam produced by scattering of the light beam having the changed local phase from the resolution volume; and
averaging the first interference pattern and the second interference pattern.

28. The method of claim 27, wherein the first interference pattern and the second interference pattern are from a plurality of interference patterns, the method further comprising:
detecting each of the plurality of interference patterns when the light beam is maintained at the beam position relative to the sample, each of the plurality of interference patterns being detected at a different time;
changing the local phase of the light beam within the resolution volume of the sample between the detecting each of the plurality of interference patterns; and
averaging each of the plurality of interference patterns.

29. The method of claim 27, wherein the changing the local phase is performed by a phase scrambler disposed within a sample light path between the light source and the sample.

30. The method of claim 29, wherein the changing the local phase includes moving the phase scrambler in a direction nonparallel to a propagation direction of the light beam.

31. The method of claim 27, wherein:
the light beam has an average wavelength; and
changing the local phase includes moving a phase scrambler within a sample light path between the light source and the sample by a distance at least as large as the average wavelength.

32. The method of claim 27, wherein:
the light beam has an average wavelength; and
changing the local phase includes moving a phase scrambler within a sample light path between the light source and the sample by a distance, the distance between about one times the average wavelength and about 10 times the average wavelength.

33. The method of claim 28, wherein:
the light beam has an average wavelength; and
changing the local phase of the light beam within the resolution volume of the sample between the detecting each of the plurality of interference patterns includes moving continuously a phase scrambler within a sample light path between the light source and the sample by a distance of less than one half the average wavelength between the detecting each of the plurality of interference patterns.

34. The method of claim 27, wherein:
the changing the local phase includes rotating a phase scrambler within a Rayleigh range of an image plane of a lens in a sample light path between the light source and the sample.

35. The method of claim 27, wherein the changing the local phase is performed by an optical element located in a Fourier domain.

36. The method of claim 27, wherein the changing the local phase is performed by a phase scrambler disposed within a sample light path between the light source and the sample, the phase scrambler including at least one of a ground glass, a sandblasted glass, an opal diffusing glass, a holographic optical element, or a spatial light modulator.

37. The method of claim 27, further comprising:
generating an image of the sample based at least in part on an averaged image of the first interference pattern and the second interference pattern.

38. A method of coherence tomography, comprising:
transmitting from a light source a reference beam portion of a spatially coherent light beam to a reference member;
transmitting from the light source a sample beam portion of the spatially coherent light beam to a resolution volume associated with a sample;
changing a local phase of at least one of the reference beam portion or the sample beam portion;
detecting, at a first time and when the sample beam portion is in a beam position relative to the sample, a first interference pattern associated with the resolution volume, the first interference pattern produced based on the reference beam portion and the sample beam portion;
changing, at a second time after the first time, the local phase of at least one of the reference beam portion or the sample beam portion;
detecting, at a third time and when the sample beam portion is in the beam position, a second interference pattern associated with the resolution volume, the second interference pattern produced based on the reference beam portion and the sample beam portion; and
averaging the first interference pattern and the second interference pattern.

39. The method of claim 38, wherein
the changing the local phase includes changing the local phase of the reference beam portion via a phase scrambler disposed within a reference light path between the light source and a reference arm.

40. The method of claim 38, wherein
the changing the local phase is performed by an optical element located in a Fourier domain within a reference light path between the light source and a reference arm.

41. The method of claim 38, wherein
the changing the local phase includes changing the local phase of the sample beam portion via a phase scrambler disposed within a sample light path between the light source and the sample.

42. The method of claim 38, wherein:
the light beam has an average wavelength; and
changing the phase is performed by moving a phase scrambler within a sample light path between the light source and the sample by a distance at least as large as the average wavelength.

43-57. (canceled)
